(19) United States
(12) Patent Application Publication
(10) Pub. No.: US 2025/0082916 A1
(43) Pub. Date: Mar. 13, 2025

(54) HEMOSTASIS AND FLUSHING METHOD AND APPARATUS FOR TRANS-CATHETER ANNULOPLASTY

(71) Applicant: Silara Medtech Inc., Santa Rosa, CA (US)

(72) Inventors: Trevor M. GREENAN, Santa Rosa, CA (US); Richard GLENN, Santa Rosa, CA (US); Travis ROWE, Santa Rosa, CA (US)

(21) Appl. No.: 18/884,022

(22) Filed: Sep. 12, 2024

Related U.S. Application Data
(60) Provisional application No. 63/582,202, filed on Sep. 12, 2023.

(51) Int. Cl. A61M 39/06; A61F 2/24; A61M 39/00; A61M 39/22; A61M 39/24

(52) U.S. Cl. CPC A61M 39/0613; A61F 2/2466; A61M 2039/0018; A61M 2039/0626; A61M 2039/064; A61M 2039/0673; A61M 39/225; A61M 2039/2426; A61M 2202/0007; A61M 2202/04; A61M 2205/0216; A61M 2205/0238; A61M 2205/025

(57) ABSTRACT

A flushing and hemostasis apparatus for a trans-catheter surgical procedure may be provided with an elongated body. A seal assembly may be located at a proximal end of the elongated body and configured to receive a distal end of an implant loading tool therethrough. A valve assembly may be located at a distal end of the elongated body and may include a flush port. In some embodiments, the valve assembly is configured to move between an open position in which the flush port is in fluid communication with the mid-section of the elongated body and a closed position in which the distal end of the elongated body is sealed off from the mid-section. A mid-section of the elongated body may be configured to receive an element protruding from the distal end of the implant loading tool. Other flushing and hemostasis tools and methods of use are also provided.

### PRIORITY CLAIM

This patent application claims the benefit of U.S. Provisional Patent Application No. 63/582,202, titled “HEMOSTASIS AND FLUSHING METHOD AND APPARATUS FOR TRANS-CATHETER ANNULOPLASTY”, and filed on Sep. 12, 2023, which is herein incorporated by reference in its entirety.

### INCORPORATION BY REFERENCE

All publications and patent applications mentioned in this specification are herein incorporated by reference for all intents and purposes to the same extent as if each individual publication or patent application was specifically and individually indicated to be incorporated by reference.

### FIELD

Embodiments of the disclosure relate generally to implanted medical devices. Specifically, some implementations of the present invention relate to apparatus and methods for repairing a mitral valve.

### BACKGROUND

The mitral valve is located at the junction between the left atrium and the left ventricle of the heart. During diastole, the valve opens, in order to allow the flow of blood from the left atrium to the left ventricle. During systole, when the left ventricle pumps blood into the body via the aorta, the valve closes to prevent the backflow of blood into the left atrium. The mitral valve is composed of two leaflets (the posterior leaflet and the anterior leaflet), which are located at the mitral annulus, the annulus being a ring that forms the junction between the left atrium and the left ventricle. The mitral valve leaflets are tethered to papillary muscles of the left ventricle via chordae tendineae. The chordae tendineae prevent the mitral valve leaflets from averting into the left atrium during systole.

Mitral valve regurgitation is a condition in which the mitral valve does not close completely, resulting in the backflow of blood from the left ventricle to the left atrium. In some cases, regurgitation is caused by dilation of the mitral annulus, and, in particular, by an increase in the anteroposterior diameter of the mitral annulus. Alternatively, or additionally, mitral regurgitation is caused by dilation of the left ventricle that, for example, may result from an infarction. The dilation of the left ventricle results in the papillary muscles consistently tethering the mitral valve leaflets into an open configuration, via the chordae tendineae.

Prior art methods and devices exist for treating mitral regurgitation. They involve either replacing or repairing the mitral valve. Replacing the valve is typically done either transapically or transseptally. Repairing the valve typically falls into one of four categories: leaflet clip; direct annuloplasty; indirect annuloplasty or chordae repair. Direct and indirect annuloplasty both involve reshaping the mitral annulus and or the left ventricle of a subject so that the anterior and posterior leaflet coapt properly. For some annuloplasty applications, a ring is implanted in the vicinity of (e.g., on or posterior to) the mitral annulus. The purpose of the ring is to reduce the circumference of the mitral annulus.

In light of the above prior art, it is desirable to provide improved systems and methods for treating mitral valve regurgitation.

### BRIEF DESCRIPTION OF THE DRAWINGS

A better understanding of the features and advantages of the present disclosure will be obtained by reference to the following detailed description that sets forth illustrative embodiments, in which the principles of the disclosure are utilized, and the accompanying drawings of which:

FIG. 1 is a generally cranial to caudal view showing aspects of a human mitral valve.

FIG. 2 is a perspective view showing an exemplary posterior bar constructed according to aspects of the present disclosure.

FIG. 3 is a top plan view showing an exemplary anterior pad constructed according to aspects of the present disclosure.

FIG. 4 is a flowchart schematically illustrating an exemplary method of performing an annuloplasty procedure according to aspects of the present disclosure.

FIGS. 5-22 are a series of perspective views looking in a generally caudal direction through a left atrium at a mitral valve and showing the steps of the exemplary method outlined in FIG. 4.

FIG. 23 is a perspective view showing a second exemplary embodiment of an annuloplasty system constructed and implanted according to aspects of the present disclosure.

FIG. 24 is a perspective view showing the annuloplasty system of FIG. 23 with some instrumentation that may be used to implant it.

FIG. 25 is a top plan view showing the plate of the posterior implant of the annuloplasty system of FIG. 23.

FIG. 26 is a perspective view showing the posterior implant of the annuloplasty system of FIG. 23.

FIG. 27 is a top plan view showing the posterior implant of FIG. 26.

FIG. 28 is a side view showing the posterior implant of FIG. 26.

FIG. 29 is a bottom view showing the posterior implant of FIG. 26.

FIG. 30 is a perspective view of an eyelet assembly of the annuloplasty system of FIG. 23.

FIG. 31 is a side view of the eyelet assembly of FIG. 30.

FIG. 32 is a top view of the eyelet assembly of FIG. 30.

FIG. 33 is a side view of a spinner assembly of the annuloplasty system of FIG. 23.

FIG. 34 is an orthogonal side view of the spinner assembly of FIG. 33.

FIG. 35 is a bottom view of the spinner assembly of FIG. 33.

FIG. 36 is a top view of the spinner assembly of FIG. 33.

FIG. 37 is a perspective view of the posterior implant of the annuloplasty system of FIG. 23 with some instrumentation that may be used to implant it.

FIG. 38 is a perspective view showing a portion of the posterior implant and instrumentation of FIG. 37 before the anchor is implanted.

FIG. 39 is a perspective view showing a portion of the posterior implant and instrumentation of FIG. 37 after the anchor is implanted and decoupled.

FIG. 40 is a perspective view showing a portion of the posterior implant and instrumentation of FIG. 37.

FIG. 41 is an enlarged perspective view showing a portion of the posterior implant and instrumentation of FIG. 40 with some of the components shown in cross-section.

FIG. 42 is a top plan view showing the plate of an anterior implant of the annuloplasty system of FIG. 23.

FIG. 43 is a perspective view showing an anterior implant of the annuloplasty system of FIG. 23 with some instrumentation that may be used to implant it.

FIG. 44 is a side view showing the posterior implant of the annuloplasty system of FIG. 23 preloaded into an implant loader.

FIG. 45 is a side view showing an anterior implant of the annuloplasty system of FIG. 23 preloaded into an implant loader.

FIG. 46 is a side view showing an anterior implant being deployed from the implant loader of FIG. 45.

FIG. 47A is a flowchart schematically illustrating a second exemplary method of performing an annuloplasty procedure according to aspects of the present disclosure.

FIG. 47B is a perspective view showing the annuloplasty system of FIG. 23 implanted across a mitral valve according to an exemplary procedure of the present disclosure.

FIG. 48A is a perspective view showing an exemplary adjustable base system.

FIG. 48B is a side elevation view showing the base system of FIG. 48A in a lowered position with a slight negative angle.

FIG. 48C is a side elevation view showing the base system of FIG. 48A in a lowered position with a slight positive angle.

FIG. 48D is a side elevation view showing the base system of FIG. 48A in a raised position with a moderate positive angle.

FIG. 48E is a side elevation view showing the base system of FIG. 48A in a raised position with a steep positive angle.

FIG. 48F is a perspective view showing an exemplary annuloplasty instrumentation system constructed according to aspects of the present disclosure.

FIG. 49A is a perspective view showing an exemplary outer steerable catheter assembly.

FIG. 49B is a side elevational view showing the proximal end of the outer steerable catheter assembly of FIG. 49A.

FIG. 49C is a longitudinal cross-sectional view of FIG. 49B.

FIG. 50A is a perspective view showing an exemplary implant loading tool assembly.

FIG. 50B is an exploded view showing the components of the tool in FIG. 50A.

FIG. 50C is an enlarged transparent view showing the proximal end of the tool in FIG. 50A.

FIG. 50D is a longitudinal cross-sectional view schematically showing aspects of the tool of FIG. 50A in use in the proximal end of an outer steerable catheter.

FIG. 51 is a perspective view showing an exemplary inner steerable catheter assembly configured for implanting a posterior implant.

FIG. 52A is an enlarged perspective view showing functions of the proximal control handle portion of the inner steerable catheter assembly of FIG. 51.

FIG. 52B is an enlarged perspective view showing the proximal control handle portion of the inner steerable catheter assembly of FIG. 51.

FIG. 53 is a perspective view showing the control handle of FIG. 52B.

FIG. 54 is a front-end elevational view showing the control handle of FIG. 52B.

FIG. 55 is a right-side elevational view showing the control handle of FIG. 52B.

FIG. 56 is a top plan view showing the control handle of FIG. 52B.

FIG. 57 is a top plan view showing an exemplary control handle configured for implanting a medial anterior implant.

FIG. 58 is a top plan view showing an exemplary control handle configured for implanting a lateral anterior implant.

FIG. 59A is an exploded perspective view showing the control handle of FIG. 52B.

FIG. 59B is a longitudinal cross-sectional view showing the control handle of FIG. 52B.

FIG. 59C is an enlarged longitudinal cross-sectional view showing a distal portion of FIG. 59B.

FIG. 59D is an enlarged longitudinal cross-sectional view showing a mid-portion of FIG. 59B.

FIG. 59E is an enlarged longitudinal cross-sectional view showing a proximal portion of FIG. 59B.

FIG. 59F is a transverse cross-sectional view of the MLE of the inner steerable catheter assembly of FIG. 51.

FIG. 59G is a transverse cross-sectional view of the MLE of the inner steerable catheter assemblies of FIGS. 57 and 58.

FIG. 60A is a view similar to FIG. 59F with the lumens populated.

FIG. 60B is a view similar to FIG. 60A with dimensions added.

FIG. 60C is a chart showing the gap dimensions between the elements of FIGS. 60A and 60B.

FIG. 60D is a perspective view showing a distal portion of the inner steerable catheter assembly of FIG. 58.

FIG. 60E is similar to the view of FIG. 60D rotated 90 degrees.

FIG. 60F is a top plan view showing the distal steerable tube of FIGS. 60D and 60E laid out flat.

FIG. 60G is a perspective view showing an exemplary guide clip.

FIG. 60H is an end view showing the guide clip of FIG. 60G.

FIG. 60I is a side view showing the guide clip of FIG. 60G.

FIG. 61 is a perspective view showing an exemplary tether retaining yoke.

FIG. 62A is a top plan view showing an exemplary bifurcated loading tool.

FIG. 62B is an exploded view showing the components of the loading tool of FIG. 62A.

FIG. 62C is a top plan view showing the loading tool of FIG. 62A.

FIG. 62D is a top plan view showing an exemplary tether guide configured for use with the loading tool of FIG. 62A.

FIG. 62E is a top plan view showing the tether guide of FIG. 62D inserted into the loading tool of FIG. 62A.

FIG. 62F is a transparent top plan view showing the combination of FIG. 62E.

FIG. 63A is a perspective view showing an exemplary tether lock constructed according to aspects of the present disclosure in an unlocked state.

FIG. 63B is a side cross-sectional view showing the tether lock of FIG. 63A in an unlocked state.

FIG. 64A is a front cross-sectional view showing the tether lock of FIG. 63A in an unlocked state.

FIG. 64B is a front cross-sectional view showing the tether lock of FIG. 63A in a locked state.

FIG. 65A is a perspective view showing the tether lock of FIG. 63A in an unlocked state.

FIG. 65B is a perspective view showing the tether lock of FIG. 63A in a locked state.

FIG. 65C is a perspective view showing the tether lock of FIG. 63A in a locked state and a sliding collar being proximally withdrawn.

FIG. 65D is a perspective view showing the tether lock of FIG. 63A in a locked state and a coupling member being uncoupled from a coupling element.

FIG. 66A is a perspective view showing a tether lock similar to the one shown in FIGS. 63A-65D.

FIG. 66B is an exploded view showing components of the tether lock of FIG. 66A.

FIG. 66C is a perspective view showing the tether lock of FIG. 66A with some components removed for ease of understanding.

FIG. 66D is a top plan view showing the tether lock of FIG. 66C.

FIG. 67 is a perspective view showing the proximal handle portion of an exemplary tensioning and locking instrument constructed according to aspects of the present disclosure.

FIG. 68 is a perspective view showing the distal end portion of the instrument of FIG. 67.

FIG. 69 is a partially exploded view of FIG. 68.

FIG. 70A is an exploded view of FIG. 67.

FIG. 70B is an enlarged perspective view showing the gear rack drive mechanism of the instrument of FIG. 67.

FIG. 71 is a longitudinal cross-sectional view of FIG. 67.

FIG. 72 is a perspective view showing the bottom of the gear rack of FIG. 70B.

FIG. 73 is a side view showing two tether tensioning and locking instruments mounted on a single rail.

FIG. 74 is a perspective view showing the proximal and distal ends of an exemplary tether cutting instrument constructed according to aspects of the present disclosure.

FIG. 75 is an enlarged, semi-transparent perspective view showing the distal end of the instrument of FIG. 74.

FIG. 76 is an exploded perspective view showing the distal end of the instrument of FIG. 74.

FIG. 77 is an exploded top plan view showing the distal end of the instrument of FIG. 74.

FIG. 78 is an enlarged transparent view showing the distal end of the instrument of FIG. 74.

FIG. 79 is an enlarged transparent view showing the distal end of the instrument of FIG. 74 with an upper stationary plate removed for ease of understanding.

FIG. 80 is an enlarged transparent view showing the distal end of the instrument of FIG. 74 with an upper stationary plate, a lower stationary plate and a blade holder removed for ease of understanding.

FIG. 81 is an enlarged transparent top plan view showing the distal end of the instrument of FIG. 74 with a cutting blade and a blade holder in a distal position.

FIG. 82 is an enlarged transparent top plan view showing the distal end of the instrument of FIG. 74 with a cutting blade and a blade holder in a distal position and an upper stationary plate and a lower stationary plate removed for ease of understanding.

FIG. 83 is an enlarged transparent top plan view showing the distal end of the instrument of FIG. 74 with a cutting blade and a blade holder in a proximal position and an upper stationary plate, a lower stationary plate and a pull pin removed for ease of understanding.

FIG. 84 is an enlarged transparent side view showing the cutting blade of the instrument of FIG. 74.

FIG. 85 is a rear side view showing the proximal end of the instrument of FIG. 74.

FIG. 86 is an exploded perspective view showing the proximal end of the instrument of FIG. 74.

FIG. 87 is a top plan view showing an exemplary flush tool constructed according to aspects of the present disclosure.

FIG. 88 is a bottom plan view showing the flush tool of FIG. 87 coupled to the distal end of an implant loading tool.

FIG. 89 is a perspective view showing an exemplary outer guide proximal handle assembly constructed according to aspects of the present disclosure.

FIG. 90 is a side cross-sectional view showing the handle assembly of FIG. 89.

FIG. 91 is a perspective cross-sectional view showing the handle assembly of FIG. 89 with an implant loading tool about to be received therein.

FIG. 92 is a perspective cross-sectional view showing the handle assembly of FIG. 89 with an implant loading tool received therein.

FIG. 93 is an exploded perspective view showing an exemplary bifurcated hemostasis tool constructed according to aspects of the present disclosure.

FIG. 94 is a perspective view showing a single slit valve of the hemostasis tool of FIG. 93.

FIG. 95 is a side elevation view showing the single slit valve of FIG. 94.

FIG. 96 is a perspective view showing an hourglass valve of the hemostasis tool of FIG. 93.

FIG. 97 is a side cross-sectional view showing the hourglass valve of FIG. 96.

FIG. 98 is an enlarged exploded perspective view showing the hemostasis tool of FIG. 93.

FIG. 99 is an exploded perspective view showing another exemplary bifurcated hemostasis tool constructed according to aspects of the present disclosure.

FIG. 100 is an exploded perspective view showing yet another exemplary bifurcated hemostasis tool constructed according to aspects of the present disclosure.

FIG. 101 is a perspective view showing the bifurcated hemostasis tool of FIG. 100.

FIG. 102 is a proximal end view showing the bifurcated hemostasis tool of FIG. 100.

FIG. 103 is a semi-transparent top plan view showing the bifurcated hemostasis tool of FIG. 100 with its valves in a Closed position.

FIG. 104 is a semi-transparent top plan view showing the bifurcated hemostasis tool of FIG. 100 with its valves in an Open position.

FIG. 105 is a partially cross-sectioned top plan view showing exemplary flushing and hemostasis features on an inner guide handle assembly constructed according to aspects of the present disclosure.

### DETAILED DESCRIPTION

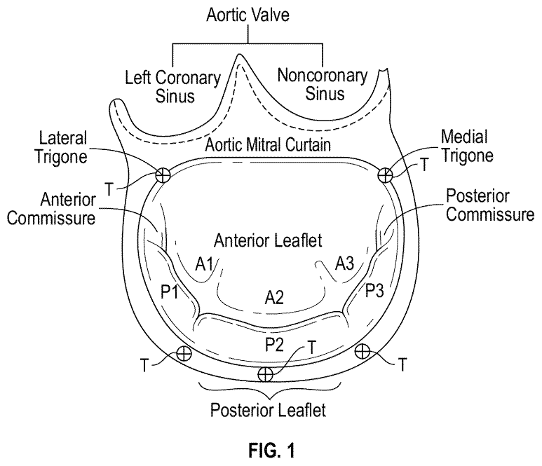

Referring to FIG. 1, elements of a mitral valve are shown. In particular, the mitral valve comprises an anterior leaflet, a posterior leaflet, an anterior-lateral commissure, a posterior-medial commissure, a lateral trigone (sometimes referred to as left) and a medial trigone (sometimes referred to as right). The anterior leaflet includes three divisions, A1, A2 and A3. Similarly, the posterior leaflet also includes three divisions, P1, P2 and P3. According to aspects of the present disclosure, in some implementations device anchors may be placed at or near each of the target locations T as shown.

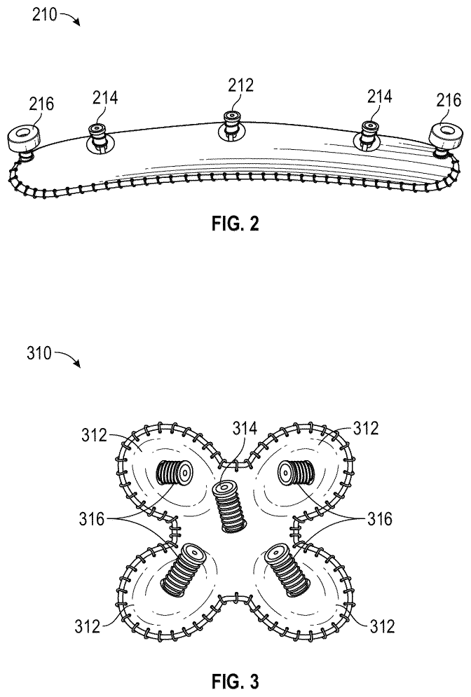

Referring to FIG. 2, an exemplary posterior bar 210 constructed according to aspects of the present disclosure is shown. Posterior bar 210 is configured to be implanted in the left atrium on or near the mitral annulus adjacent to the posterior leaflet, as will be subsequently described in more detail. As such, in this exemplary embodiment, posterior bar 210 is an elongated tubular structure that is curved to match the anatomy of the mitral annulus in this location. Posterior bar 210 may be provided with a low profile as shown to minimize the amount of irregular structure in the atrium that might be a potential site for thrombosis. In this exemplary embodiment, posterior bar 210 is provided with atraumatic edges to limit the potential for tissue damage, and is covered in polyethylene terephthalate (PET) fabric to aid with tissue ingrowth.

In this exemplary embodiment, posterior bar 210 is provided with a middle tissue anchor guide 212 and two end tissue anchor guides 214. In some embodiments, middle tissue anchor guide 212 is identical to end tissue anchor guides 214, and in other embodiments it is configured differently, such as having features that facilitate the steering/torquing of posterior bar 210 during delivery. In some embodiments, as will be subsequently described herein, there may be no middle tissue anchor guide, and there may be greater or fewer than the three tissue anchor guides provided in this exemplary embodiment. Anchor guides 212 and 214 may be configured to pivot relative to posterior bar 210 such that they can move from a retracted state and a deployed state. In the retracted state, anchor guides 212 and 214 may extend generally parallel to bar 210 so that they and bar 210 may together pass through a lumen of a catheter. In the deployed state, anchor guides 212 and 214 may extend generally perpendicular to bar 210 as shown in FIG. 2 so that they may be used to thread a tissue anchor over the guide, through apertures in bar 210 and into adjoining tissue to secure bar 210 to the tissue.

One or more snare features 216 may be provided on posterior bar 210. In this exemplary embodiment, two snare features 216 are provided, one near each end of posterior bar 210. Snare feature 216 may be configured to prominently extend from posterior bar 210 such that they can easily engage with one or more tensile members/snares, and also to prevent the tensile members from disengaging during manipulation. In some embodiments, snare features 216 are configured to be easily imaged under fluoroscopy and echocardiography to aid in positioning posterior bar 210 during delivery and attachment to tissue, and to aid in connecting tensile members to the snare features 216.

Posterior bar 210 may be designed to preferentially load anchors in shear versus tension with respect to the anatomy. Torque control features may be provided to allow the initial positioning of posterior bar 210, and to allow the ability to move the implant as subsequent anchors are delivered to match the anatomy.

Posterior bar 210 may also be provided with some level of flexibility to allow for in vivo adjustment of the bar to contour to the particular subject's anatomy. The flexibility of posterior bar 210 may also serve to allow the bar to flex during the cardiac cycle. In some embodiments, the flexibility of posterior bar 210 is created by providing a series of slits (not shown in FIG. 1) transverse to the longitudinal axis of the bar. In some embodiments, the slits and or other flexibility-providing features may be configured to limit the minimum radius of posterior bar 210 when implanted to ensure it applies a more uniform tension to the posterior side of the mitral annulus.

Referring to FIG. 3, an exemplary anterior pad 310 constructed according to aspects of the present disclosure is shown. Anterior pad 310 is configured to be implanted in the left atrium on or near the mitral annulus adjacent to the anterior leaflet, particularly on a trigone, as will be subsequently described in more detail. In this exemplary embodiment, anterior pad 310 is a generally flat structure provided with four petals 312 radially extending from a central portion. In other embodiments, more, fewer or no petals may be provided. A primary tissue anchor 314 may be located in the center of anterior pad 310. In some embodiments, additional tissue anchors 316 may be provided, such as an additional anchor 316 near the center of each petal 312, as shown. In some embodiments, primary tissue anchor 314 is identical to additional tissue anchors 316, and in other embodiments it is configured differently, such as having features that facilitate the positioning of anterior pad 310 during delivery. The petals 312 may be designed to fold into a compact configuration such that anterior pad 310 may be delivered through a catheter.

Anterior pad 310 may be provided with a low profile as shown to minimize the amount of irregular structure in the atrium that might be a potential site for thrombosis. In this exemplary embodiment, anterior pad 310 is provided with atraumatic edges to limit the potential for tissue damage, and is covered in polyethylene terephthalate (PET) fabric to aid with tissue ingrowth.

One or more snare features may be provided on anterior pad 310. In this exemplary embodiment, the top ends of tissue anchors 314 and 316 are configured to engage with one or more tensile members/snares These snare features may be configured to prominently extend from anterior pad 310 such that they can easily engage with one or more tensile members/snares, and also to prevent the tensile members from disengaging during manipulation. In some embodiments, the snare features and or the entire anterior pad 310 are configured to be easily imaged under fluoroscopy and echocardiography to aid in positioning anterior pad 310 during delivery and attachment to tissue, and to aid in connecting tensile members to the snare features. Anterior pad 310 may be designed to preferentially load anchors in shear versus tension with respect to the anatomy.

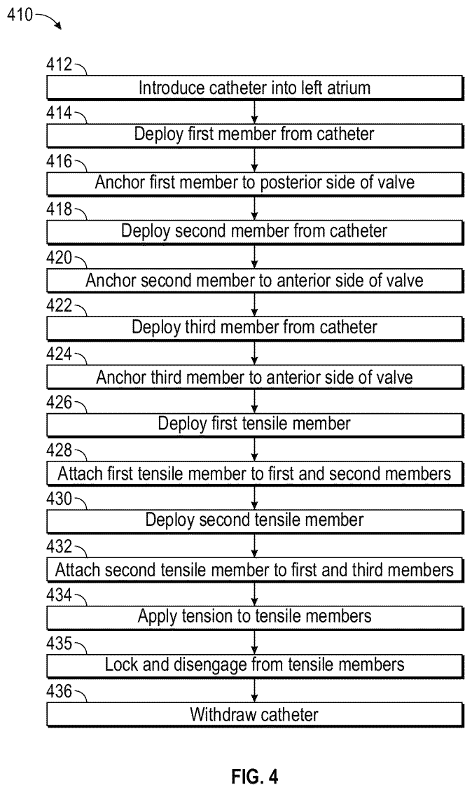

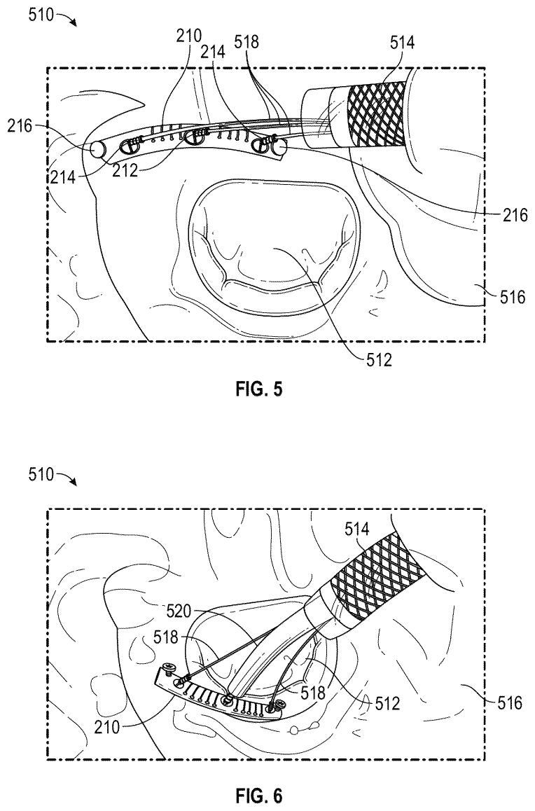

Referring to FIG. 4, an exemplary method of performing an annuloplasty procedure according to aspects of the present disclosure is shown. The steps of this exemplary method 410 will be described in reference to the flowchart shown in FIG. 4 and the series of images shown in FIGS. 5-22. In each of the images shown in FIGS. 5-22, the view is looking in a generally caudal direction through the left atrium 510 toward the mitral valve 512 with the medial direction generally to the right. In some implementations of the method, one posterior bar 210 and one, two or more anterior pads 310 are implanted. In other implementations, different types or numbers of devices may be used. In FIGS. 5-22, posterior bar 210 is shown without a fabric cover for clarity. In this exemplary embodiment, at least one device anchor is placed at or near each of the five target locations T shown in FIG. 1.

In some implementations of method 410, the first step 412 of the method is introducing the distal end of a delivery catheter into the left atrium 510 of a subject. This may be performed using a transseptal approach, a left atrial approach or other methodology for gaining access to the left atrium. In the images shown in FIGS. 5-22, a transseptal approach is depicted with the distal end of catheter 514 passing through the septum 516 of the heart and into the left atrium 510 of the subject. In some implementations, an inner dilator (not shown) is located in the distal end of catheter 514 for crossing the septum.

Referring to FIGS. 4 and 5, once the distal end of catheter 514 is introduced into the left atrium 510, a posterior bar 210, sometimes referred to herein as a first member, may be deployed from the distal end of catheter 514 in step 414. In some implementations, catheter 514 is first introduced into the left atrium 510 before the posterior bar assembly is loaded into the proximal end of the catheter 514. In other implementations the posterior bar 210 along with its tissue anchor guides 212, 214 and snare features 216 may be pre-loaded into catheter (not shown) and advanced through catheter 514. As seen in FIG. 5, an anchor lead 518 may be removably attached to each of the tissue anchor guides 212 and 214 to push posterior bar 210 through catheter 514 and deploy it from the distal end.

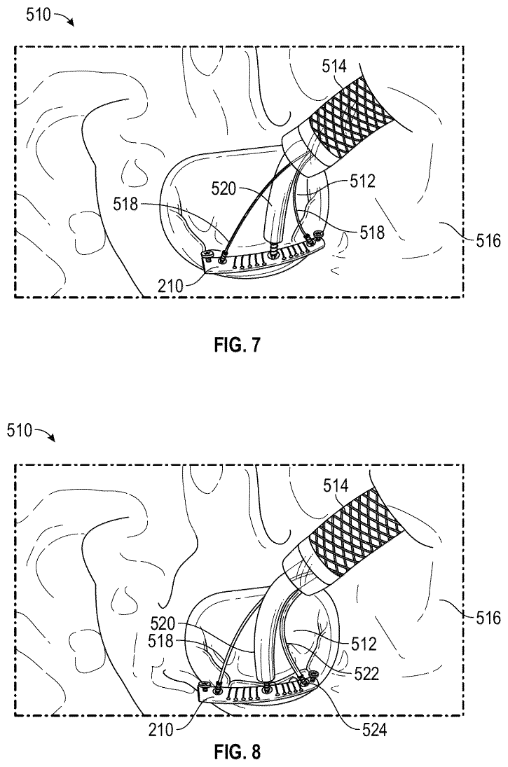

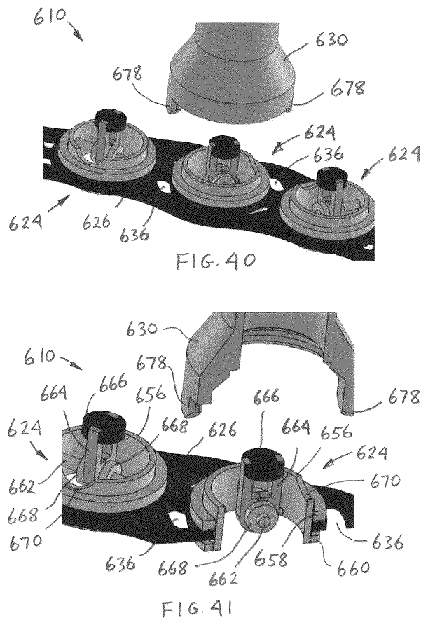

Referring to FIG. 6, once posterior bar 210 emerges from the distal end of catheter 514, a lead 518 attached to one of its ends may be pushed and the other pulled from the proximal end of catheter 514 to pivot posterior bar 210 into an orientation that is generally perpendicular to catheter 514, as shown. A steerable inner catheter 520 may be slid distally over middle lead 518 until features (such as recesses and/or castellations, not shown) engage with mating features on posterior bar 210 to keep bar 210 from rotating relative to inner catheter 520. Steerable inner catheter 520 may then be used to position and rotate posterior bar 210 until it is steered into its desired implantation location and orientation, as shown in FIG. 7. In some implementations, a torque driver coaxially located between lead 518 and steerable inner catheter 520 may be used to impart torque to posterior bar 210. Such an implementation is subsequently described in relation to FIGS. 40-46.

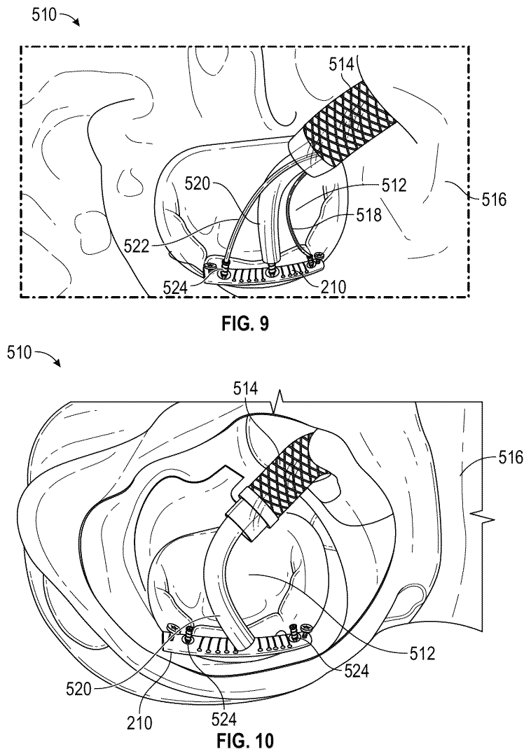

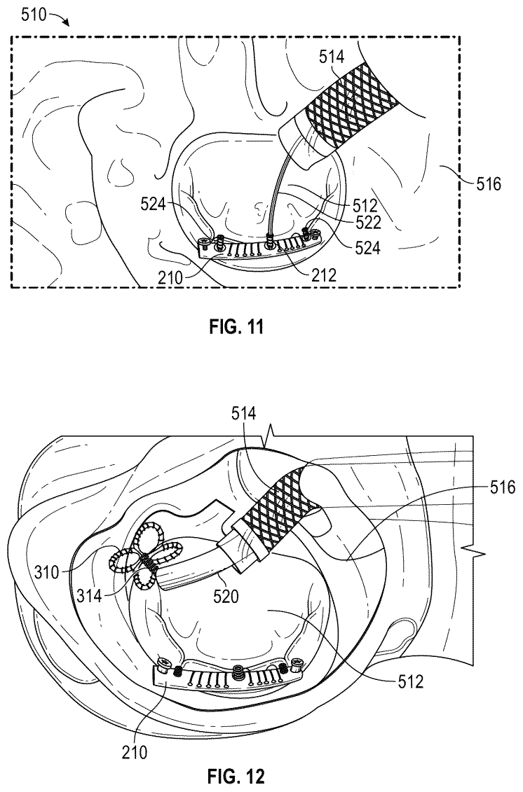

Referring to FIGS. 4 and 8-11, step 416 of exemplary method 410 will be described. In this step, posterior bar 210 (i.e., the first member) is anchored to the posterior side of mitral valve 512. This may be accomplished by first sliding a drive tube 522 with a helical tissue anchor 524 located on its distal end over lead 518 attached to the tissue anchor guide 214 located near the medial end of posterior bar 210, as shown in FIG. 8. While steerable inner catheter 520 holds posterior bar 210 against the mitral valve annulus tissue, drive tube 522 may be rotated to screw medial anchor 522 through posterior bar 210 and into the underlying tissue, as seen in FIG. 9. Drive tube 522 may then be removed from the medial anchor 214 and it (or another drive tube 522 with another helical tissue anchor 524) may be slid over lead 518 attached to the tissue anchor guide 214 located near the lateral end of posterior bar 210, as shown in FIG. 9. While medial anchor 524 and steerable inner catheter 520 (and in some implementations a torque driver inside catheter 520) hold posterior bar 210 against the mitral valve annulus tissue, drive tube 522 may be rotated to screw lateral tissue anchor 524 through bar 210 and into the underlying tissue, as seen in FIG. 10. Drive tube 522 may then be removed from the lateral anchor 524 and it (or another drive tube 522 with another helical tissue anchor 524) may be slid over lead 518 attached to the middle tissue anchor guide 212, as shown in FIG. 11. In some implementations, steerable inner catheter 520 may remain in place against posterior bar 210 when the central anchor is being placed (as shown in FIG. 10), or it may be removed from posterior bar 210 prior to drive tube 522 and middle anchor 524 being slid into engagement over middle tissue anchor guide 212 (as shown in FIG. 11.) While medial and lateral anchors 524 hold posterior bar 210 against the mitral valve annulus tissue, drive tube 522 may be rotated to screw middle anchor 524 through bar 210 and into the underlying tissue. FIGS. 10 and 11 show posterior bar 210 with the leads removed from the end tissue anchor guides, such as by unthreading.

In step 416, it should be noted that after the initial anchor has been placed, torque control of the implant 210 provided by steerable inner catheter 520 (or in some implementations a torque driver located within catheter 520) can be used to guide the placement of subsequent anchors to implant 210. This eliminates the need for unguided anchor placement after the initial anchor has been placed. FIG. 12 shows posterior bar 210 with the three anchors placed and all leads removed.

Referring to FIGS. 4 and 12, step 418 of exemplary method 410 will be described. In this step, anterior pad 310 (sometimes referred to herein as a second member) is deployed from the distal end of catheter 514. In some implementations, anterior pad 310 is steered toward the lateral trigone with steerable inner catheter 520 as shown in FIG. 12. (The lateral trigone is also shown in FIG. 1.)

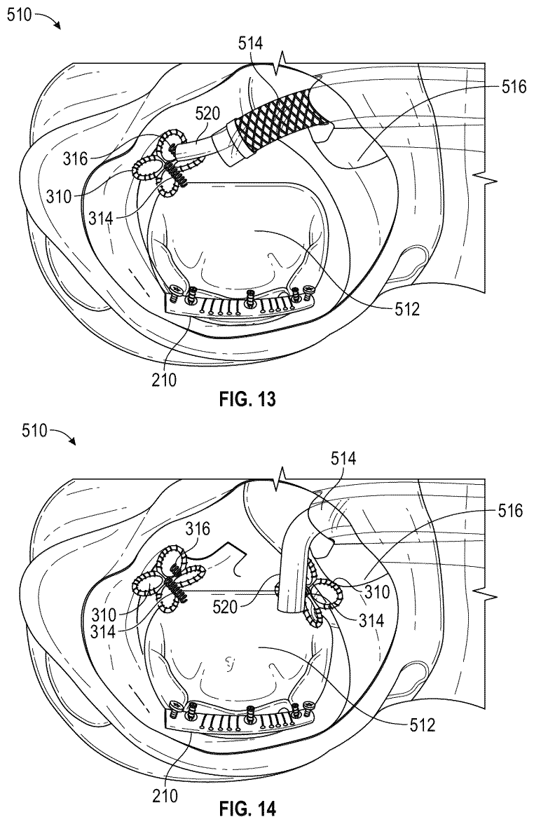

Referring to FIGS. 4, 12 and 13, step 420 of exemplary method 410 will be described. In this step, anterior pad 310 (sometimes referred to herein as a second member) is anchored to the anterior side of mitral valve 512. In some implementations, anterior pad 310 is anchored to the lateral trigone as shown with a single anchor 314. A drive tube (not shown) may be used within steerable inner catheter 520 to screw anchor 314 into place. As shown in FIG. 13, additional anchor(s) 316 may be used to further secure anterior pad 310 to the lateral trigone.

In step 420, it should be noted that after the initial anchor has been placed, its lead can remain in place through steerable inner catheter 520 such that the lead and catheter 520 can be used to guide the placement of subsequent anchors to implant 310. This eliminates the need for unguided anchor placement after the initial anchor has been placed.

Referring to FIGS. 4 and 14, steps 422 and 424 of exemplary method 410 will be described. In these steps, another anterior pad 310 (sometimes referred to herein as a third member) is deployed from the distal end of catheter 514. In some implementations, anterior pad 310 is steered toward the medial trigone with steerable inner catheter 520 as shown in FIG. 14. (The medial trigone is also shown in FIG. 1.) Anterior pad 310 may then anchored to the anterior side of mitral valve 512. In some implementations, anterior pad 310 is anchored to the medial trigone as shown with a single anchor 314. A drive tube (not shown) may be used within steerable inner catheter 520 to screw anchor 314 into place. As with the lateral anterior pad 310, additional anchor(s) may be used to further secure the medial anterior pad 310 to the medial trigone.

In step 424, it should be noted that after the initial anchor has been placed, its lead can remain in place through steerable inner catheter 520 such that the lead and catheter 520 can be used to guide the placement of subsequent anchors to implant 310. This eliminates the need for unguided anchor placement after the initial anchor has been placed.

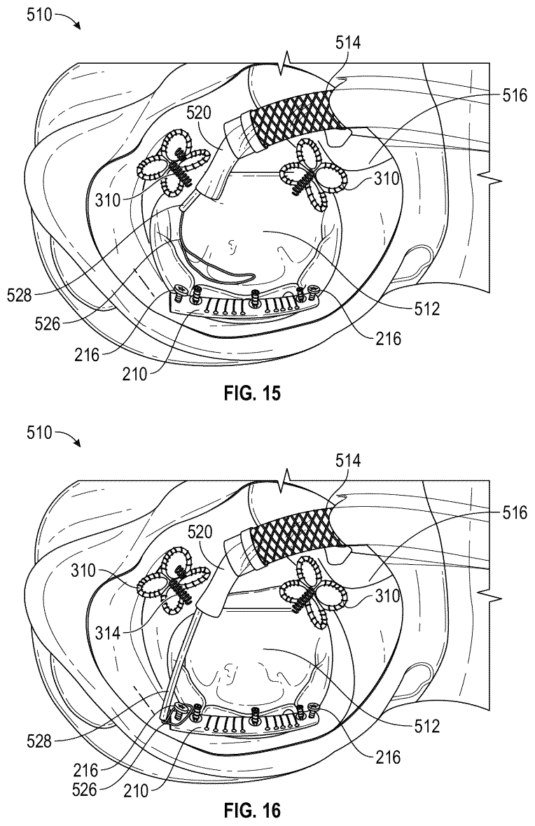

Referring to FIGS. 4 and 15, step 426 of exemplary method 410 will be described. In this step, a first tensile member, tether or snare 526 is deployed from the distal end of catheter 514 through steerable inner catheter 520 as shown. A snare sheath 528 may be used to direct the first tensile member 526 toward implant features. Snare sheath 528 may also be used to tighten first tensile member 526 around the implant features by pulling proximally on the tensile member 526 relative to the sheath 528.

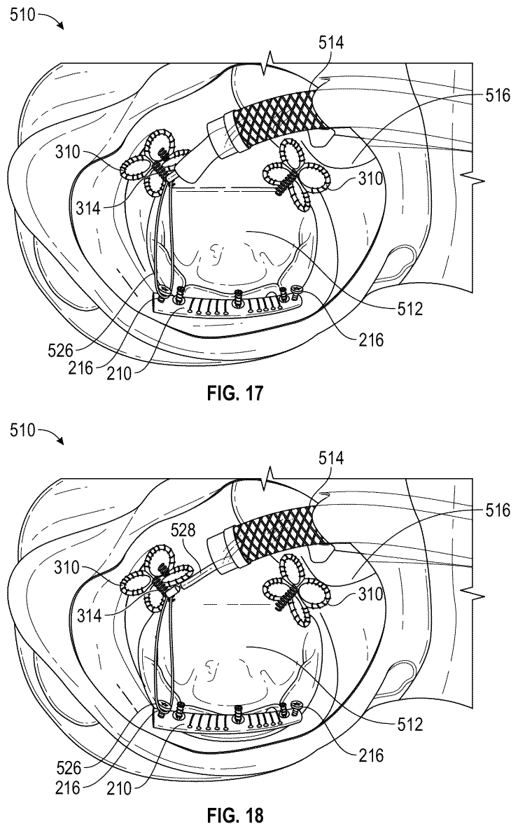

Referring to FIGS. 4 and 16-18, step 428 of exemplary method 410 will be described. In this step, first tensile member or snare 526 is attached to the posterior bar 210 (i.e., the first member) and anterior pad 310 (i.e., the second member.) Steerable inner catheter 520 and snare sheath 528 may be utilized to guide first tensile member 526 over the lateral snare feature 216 of bar 210, as shown in FIG. 16. First tensile member 526 may then be guided over primary tissue anchor 314 of anterior pad 310, as shown in FIG. 17. A small amount of tension may then be applied to first tensile member 526 with snare sheath 528 to keep it engaged with bar 210 and pad 310, as shown in FIG. 18.

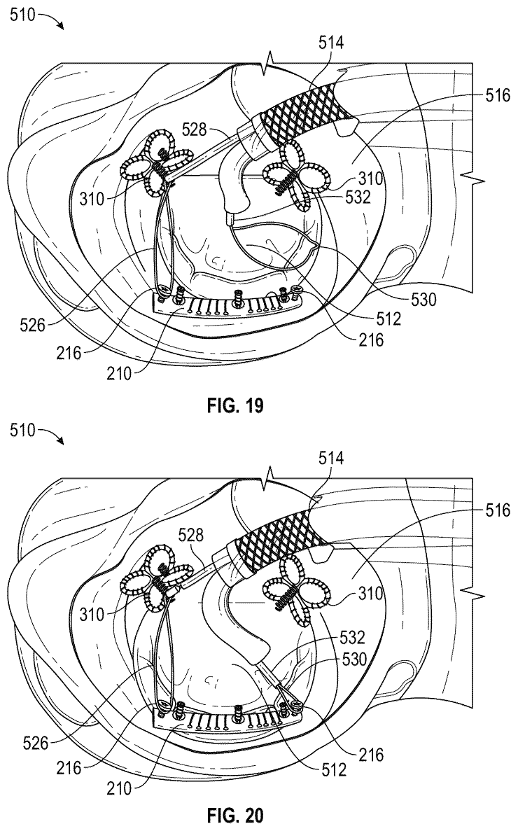

Referring to FIGS. 4 and 19, step 430 of exemplary method 410 will be described. In this step, a second tensile member or snare 530 is deployed from the distal end of catheter 514 through steerable inner catheter 520 as shown. A snare sheath 532 may be used to direct the second tensile member 530 toward implant features. Snare sheath 532 may also be used to tighten second tensile member 530 around the implant features by pulling proximally on the tensile member 530 relative to the sheath 532.

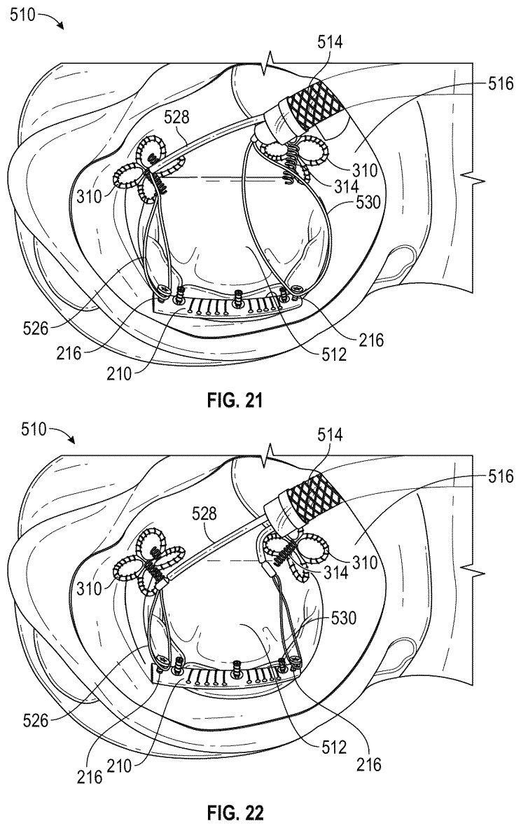

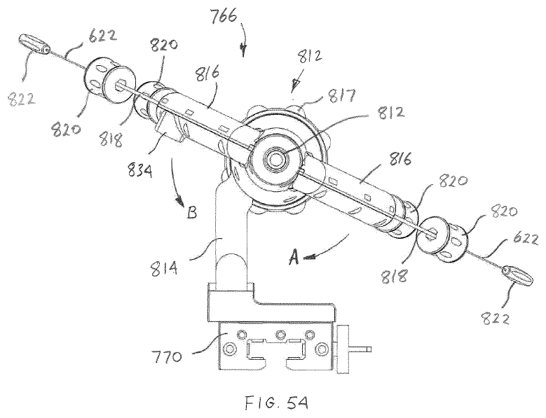

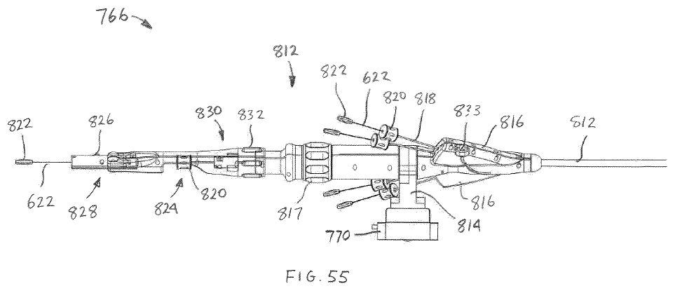

Referring to FIGS. 4 and 20-22, step 432 of exemplary method 410 will be described. In this step, second tensile member or snare 530 is attached to the posterior bar 210 (i.e., the first member) and the next anterior pad 310 (i.e., the third member.) Steerable inner catheter 520 and snare sheath 532 may be utilized to guide second tensile member 530 over the medial snare feature 216 of bar 210, as shown in FIG. 20. Second tensile member 530 may then be guided over primary tissue anchor 314 of anterior pad 310, as shown in FIG. 21. A small amount of tension may then be applied to second tensile member 530 with snare sheath 532 to keep it engaged with bar 210 and pad 310, as shown in FIG. 22. In some implementations, the snare shape may be configured to more easily engage the snare features on the implants. For example, each snare may form a D-shape that makes contact with the lateral side or medial side of the atrium. The wall of the atrium is then used to guide the snare down to the annulus and then cinch without necessarily needing to guide the snare to each snare feature. In some embodiments, the snare has a dumbbell (or dog bone) shape, such as the exemplary snare 550 shown in FIG. 54. Snare 550 includes a distal loop 552 and a proximal loop 554 having predefined diameter(s), with the rest of the snare having generally parallel tensile members forming a smaller gap between them than the loop diameter(s). Distal loop 552 may first be exposed to engage a first snare feature on an implant, and subsequently the proximal loop 554 may be exposed to capture a second snare feature on an implant. In some embodiments, as depicted in FIG. 55, two snares 560 and 562 are loaded in parallel, each with a predefined shape. Individual snares 560 and 562 may be connected with a coupler 564 and can slide independently to engage snare features on implants separately.

Once both first tensile member 526 and second tensile member 530 are in place, additional tension may be applied to both to draw the anterior and posterior sides of mitral valve 512 into closer approximation. In some implementations, tension in members 526 and 530 may be increased simultaneously. In some implementations, tension may be increased incrementally in members 526 and 530, alternating between the two until the desired tensions and or valve approximation is reached. In some implementations, the final tension and or tissue approximation of each tensile member 526 and 530 is approximately the same. In some implementations, the final tension and or tissue approximation of each tensile member 526 and 530 is different. Because medial and lateral cinching can be performed independently, the placement of each bar is more forgiving. This generally holds true for all of the systems disclosed herein. In some implementations, real time echocardiography of the mitral valve is used to monitor the reduction in mitral regurgitation as tensile members 526 and 530 are tightened.

After the desired tensions and or tissue approximations are obtained, tensile members 526 and 530 may be tied off. In some implementations, a reversible lock may be used during the cinching process which is configured to permanently hold the position of the tensile member. A disconnect member may be used to decouple the snare from the delivery system, or a portion of the tensile member may be cut to release it. Catheter 514 may then be withdrawn from the left atrium, along with steerable inner catheter 520 and snare sheaths 528 and 532 (step 436 shown in FIG. 4.) In addition to tensioning the device during the de novo procedure, additional tensioning devices can be added at a later time or date, and or the existing devices can be re-tensioned to further reduce the A-P dimension.

Additional embodiments of the preceding system and method can be found in Applicant's co-pending U.S. Patent Application Publication 2021/0052387, entitled Annuloplasty Systems and Methods.

Referring to FIGS. 23-47B, a second exemplary embodiment of an annuloplasty system 600 constructed and implanted according to aspects of the present disclosure is shown. Referring first to FIG. 23, annuloplasty system 600 is constructed and functions in a manner similar to the previously described system. It too includes an elongated posterior implant 610 configured to be implanted in the left atrium on or near the mitral annulus adjacent to the posterior leaflet, and two anterior implants 612 each configured to be implanted in the left atrium on or near the mitral annulus adjacent to the anterior leaflet, particularly on a trigone. However, in this second exemplary embodiment, instead of attaching tensile members or tethers to the implants after they have been deployed, tethers 614 are pre-attached to anterior implants 612 before they are deployed from a catheter. The anterior implants 612 are implanted first in this embodiment, and their tethers 614 are threaded through the posterior implant 610 before it is deployed from its catheter. The posterior implant 610 is then deployed from its catheter and tracks over tethers 614 as it is placed on the posterior side of the mitral annulus. After the posterior implant 610 is secured in place with anchors 616, tethers 614 can be tensioned and secured with locks 618, as will be subsequently described in detail. This arrangement saves considerable time during the procedure, not needing to snare each of the implants with the tethers. It also ensures the tether attachment points are more consistent and reliable.

In this second exemplary embodiment, posterior implant 610 is provided with five anchors 616 and anterior implants 612 are each provided with two anchors 616. Each end of posterior implant 610 is provided with a swiveling eyelet assembly 620 for tracking over a tether 614 and providing a backstop for a tether lock 618.

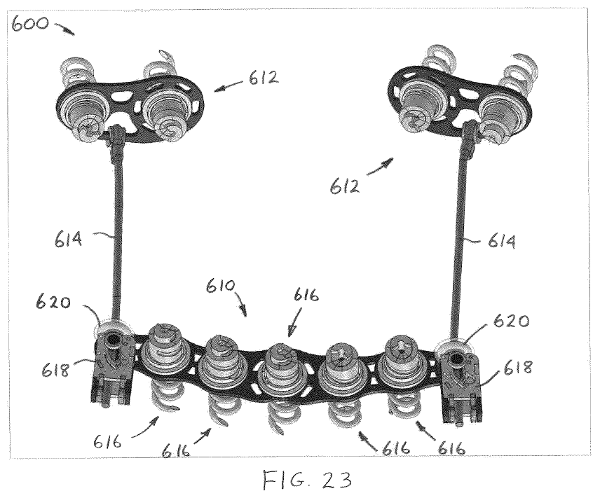

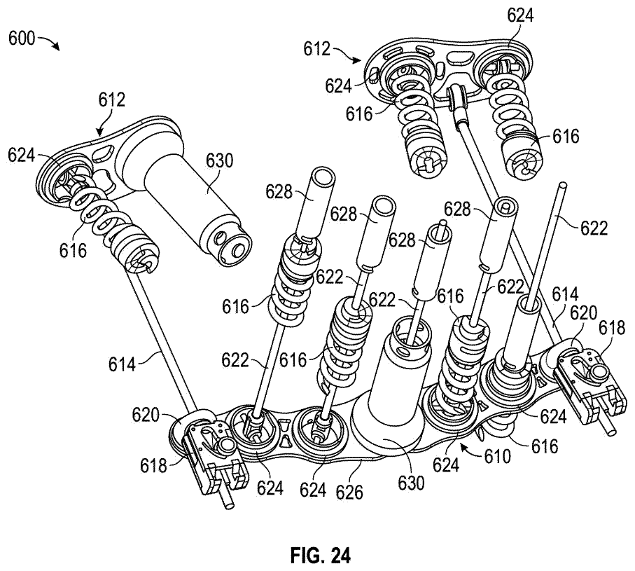

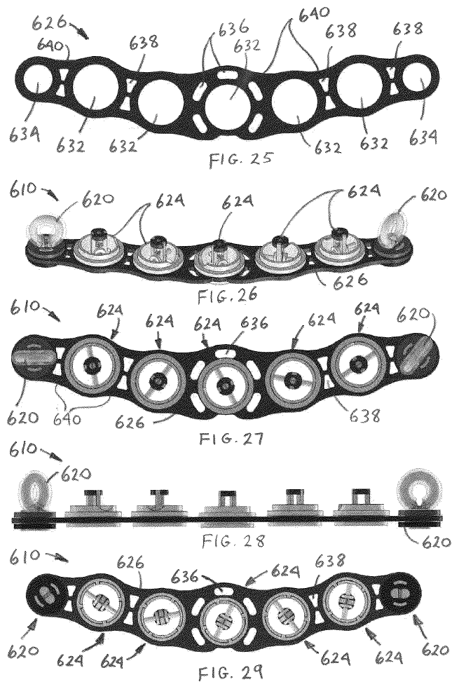

Referring to FIG. 24, another perspective view of system 600 is provided, showing anchors 616 in various stages of insertion. Each anchor 616 is guided into place by its own lead 622 detachably connected to a spinner assembly 624. Each spinner assembly 624 is rotatably attached to posterior implant plate 626. A separate driver head 628 is detachably connected to the top of each anchor 616. When a driver head 628 is rotated by a proximally extending driver tube (not shown), the attached anchor 616 is driven through its spinner assembly 624 and into the underlying heart tissue until it seats against its spinner assembly 624, thereby securing implant plate 626 against the tissue. A torque head 630 is provided for each implant (only two are shown in FIG. 24.) Each torque head 630 is longitudinally and rotationally driven by a proximally extending torque tube (not shown) to engage with its respective implant and drive the implant into position for anchoring.

Referring to FIG. 25, a bare posterior implant plate 626 is shown without any components attached for clarity. It is provided with five through holes 632 for rotatably retaining spinner assemblies 624 (shown in FIGS. 23 and 24.) Two holes 634 are also provided for rotatably retaining eyelet assemblies 620 (shown in FIGS. 23 and 24.) A series of slots 636 spaced around the central spinner assembly hole 632 are provided for engaging with torque head 630 (shown in FIGS. 23 and 24), as will be subsequently described in detail. Additional through holes 638 and scallops 640 may be provided as shown to reduce the amount of metal in plate 626 for better echo imaging and tissue ingrowth.

Referring to FIGS. 26-29, posterior implant plate 626 is shown with anchor spinner assemblies 624 and tether eyelet assemblies 620 attached to base plate 626. FIG. 26 is a perspective view, FIG. 27 is a top plan view, FIG. 28 is a side view and FIG. 29 is a bottom view.

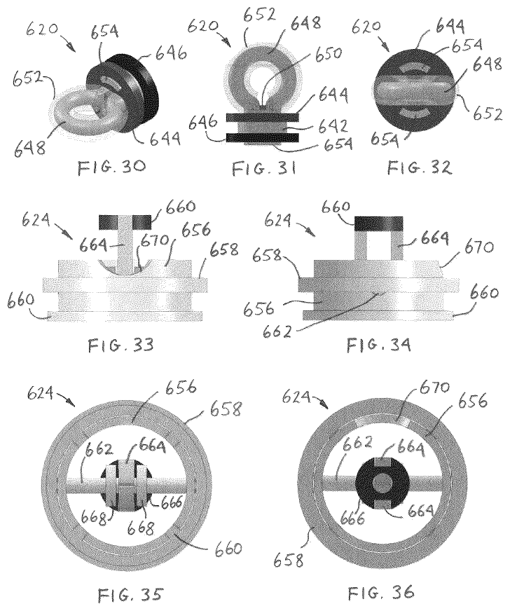

Referring to FIGS. 30-32, various views of eyelet assembly 620 are shown. FIG. 30 is a perspective view, FIG. 31 is a side view and FIG. 32 is a top plan view. (A bottom view of eyelet assembly 620 is provided in FIG. 29.) As best seen in FIG. 31, eyelet assembly 620 may be formed from six separate components: a cylindrical core 642, a top ring 644, a bottom ring 646, an eyelet 648, a wedge or filler material 650 and an eyelet covering 652. As best seen in FIGS. 31 and 32, core 642 may be provided with two pairs of arcuate fins 654, one pair protruding from the top of core 642 and one pair protruding from the bottom. Top ring 644 and bottom ring 646 may each be provided with mating slots for receiving the arcuate fins 654. In some embodiments, fins 654 are swaged, welded, epoxied, press-fit and or fastened to rings 644 and 646 by other suitable means. In other embodiments, there is only a sliding fit between fins 654 and rings 644 and 646, and the rings are held in place by being sandwiched between core 642 and the ends of a straight shank portion of eyelet 648, and or by the eyelet shank expanding against the rings, as will be described next. In some embodiments, fins 654 may serve as centering features for core 642, and or as anti-rotation features such that rings 644 and 646 do not rotate relative to core 642 and or eyelet 648. In other embodiments (not shown), feature shapes other than arcuate fins may be used.

Eyelet 648 may be formed with a straight shank section that has an oval or oblong transverse cross-section (best seen in FIG. 29.) Core 642 may be provided with a central bore having a mating oval or oblong transverse cross-section or a circular cross-section for receiving the eyelet shank. The straight shank section may be split down the center, with a gap between the two halves. This allows an anti-friction tube 652 to be slid over one half of the shank and onto the circular portion of eyelet 648, as shown. In some embodiments, tube 652 is made of or coated with polytetrafluoroethylene (PTFE) to reduce friction between eyelet 648 and the tether that passes through it. In other embodiments, eyelet 648 may be dipped directly into PTFE or another friction-reducing coating.

During assembly, the straight shank section of eyelet 648 may be passed through top ring 644, the center of core 642 (which resides in one of the holes 634 of posterior implant plate 626, shown in FIG. 25) and through bottom ring 646. A wedge or filler material 650 may then be placed between the two halves of the eyelet shank such that they are urged outwardly against the inside walls of oval or oblong bores within rings 644 and 646. In some embodiments, material 650 is a resiliently compressible material placed in the shank gap before assembly, such that it can be compressed during assembly and then exert a resilient outward force after assembly. In other embodiments, material 650 is a metal (i.e., non-compressible.) In some embodiments, the eyelet shank is provided with a necked-down portion (not shown) having an axial length slightly longer than the distance between the top of top ring 644 and the bottom of bottom ring 646. This arrangement allows core 642 and rings 644 and 646 to be captivated within the necked-down portion after the split shank is radially compressed, passed through the other components and then radially expanded. Once eyelet assembly 620 is assembled, posterior implant plate 626 (shown in FIG. 25) is sandwiched between top ring 644 and bottom ring 646 of eyelet assembly 620. Core 642 may be provided with a height that is slightly larger than the thickness of implant plate 626 such that eyelet assembly 620 may freely rotate relative to plate 626.

Each of the exemplary embodiments described above provide eyelet assembly 620 with the ability to rotate relative to posterior implant 610, thereby allowing the tether passing through the eyelet assembly 620 to align with a central lumen of a catheter during delivery and then rotate to align with an anterior trigone implant 612 once implanted.

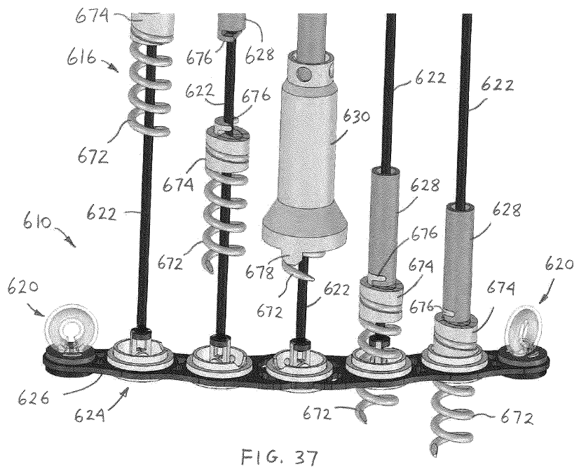

Referring to FIGS. 33-36, various views of spinner assembly 624 are shown. FIG. 33 is a side view, FIG. 34 is another side view, taken in a direction orthogonal to that of FIG. 33, FIG. 35 is a bottom view and FIG. 36 is a top view. Spinner assembly 624 may be formed from six separate components: a central hoop 656, a top disc 658, a bottom lock ring 660, a crossbar 662, a U-shaped connecting rod 664 and a lead nut 666. Central hoop 656 is configured to be rotatably received within one of holes 632 in posterior implant plate 626 (shown in FIG. 25.) Disc 658 and ring 660 may each be welded or otherwise connected to hoop 656 to rotatably captivate implant plate 626 therebetween. Crossbar 662 spans across a central bore in hoop 656 (parallel to plate 626.) U-shaped connecting rod 664 is pivotably mounted to crossbar 662, as best seen in FIG. 35 (and also shown in FIG. 41.) Lead nut 666 can be welded or otherwise fastened to the top of connecting rod 664. Lead nut 666 is provided with a central threaded bore for receiving a threaded end of an anchor lead 622 (shown in FIGS. 24 and 37.) Fixed flanges 668 may be provided on crossbar 662 to keep connecting rod 664 and lead nut 666 centered within hoop 656. Connecting rod 664 may be laser cut in an open (V-shaped) configuration and then closed (made into a U-shape) around crossbar 662 between flanges 668 before nut 666 is attached to the tops of its two prongs.

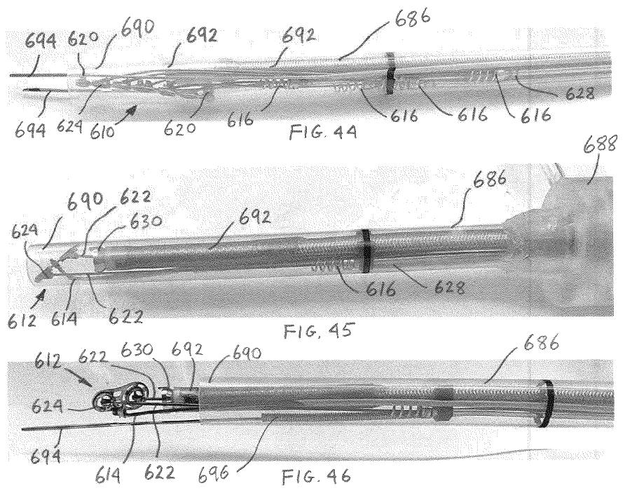

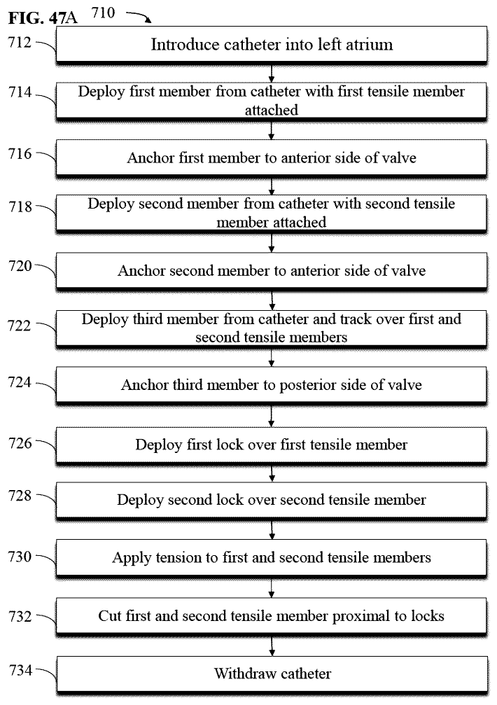

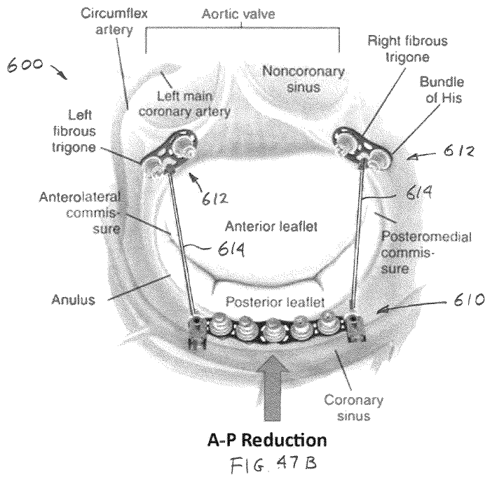

With the above-described arrangement, lead nut 666 may pivot with respect to spinner assembly 624, which in turn spins with respect to posterior implant 610 (shown in FIGS. 23, 24 and 37.) This allows an anchor lead 622 to generally lay flat against implant 610 when it is preloaded into a delivery catheter (as shown in FIG. 44), and to extend orthogonally or at another angle when the implant is being deployed. As best seen in FIGS. 33 and 40, one or more recesses 670 may be provided in the top of hoop 656 to allow connecting rod 664, lead nut 666 and anchor lead 622 (shown in FIGS. 24 and 37) to lay flatter against the implant.

Another advantage of spinner assemblies 624 is that they ensure that anchors 616 (shown in FIGS. 23 and 24) which thread through them are able to pull the implant all the way against the heart tissue without leaving any gaps between the tissue and the implant. The same spinner assemblies 624 may also be used with the two anterior implants 612 and work in much the same way as they do with posterior implant 610. As shown in FIGS. 23 and 24, a spinner assembly is provided for each anchor 616, so there are five spinner assemblies mounted on posterior implant 610 and two on each of the two anterior implants 612.

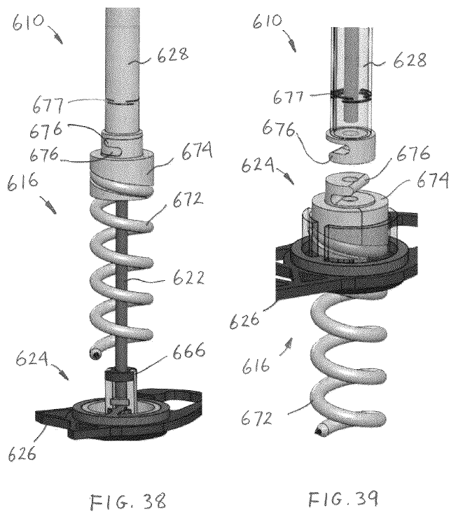

Referring to FIGS. 37-39, construction and operation of implantable anchors 616 will be described. In this exemplary embodiment, each anchor 616 is constructed from two components: a coil 672 and an anchor head 674. The distal end of anchor head 674 may be provided with a helical slot for receiving the proximal end of coil 672. In some embodiments, the proximal end of coil 672 is welded to head 674. In this exemplary embodiment, the center of anchor head 674 is hollow so that it fits over lead nut 666 and connecting rod 664 when anchor 616 is being implanted. The proximal end of anchor head 674 may be provided with a cylindrical, hook-shaped releasable engagement feature or clasp 676. An identical and or complementary mating feature or clasp 676 may be located on the distal end of driver head 628. When the implants are assembled and pre-loaded into delivery catheters, the two clasps 676 may be interlocked with each other and held together by an anchor lead 622. When interlocked, as shown in FIG. 38, clasps 676 transmit axial and rotation motion from driver head 628 to anchor 616 for driving the anchor through spinner assembly 624 and into the underlying heart tissue. After all the anchors 616 of an implant are installed, the distal end of each anchor lead 622 may be unscrewed from its associated lead nut 666 and withdrawn proximally through clasps 676 to allow each anchor driver to be disengaged from its anchor 616, as shown in FIG. 39. As shown in FIGS. 38 and 39, a series of slots or laser cuts 677 may be formed through the wall thickness of driver heads 628 to form flexures or living hinges. These flexures can relieve pressure and allow clasps 676 to more easily engage and disengage from one another when there is axial misalignment, or a lateral moment being applied to driver head 628. In other embodiments (not shown), a hollow stranded cable may be used instead of a rigid tube with or without flexures.

As also depicted in FIG. 37, torque head 630 may be provided with a flared distal end configured to fit over the central spinner assembly 624 when distally extending tabs 678 fit into slots 636 for steering implant 610. Torque head 630 may also be provided with a central bore large enough for an anchor 616 to be housed in when it is being installed.

Referring to FIGS. 40 and 41, additional views of torquer head 630 are shown. FIG. 40 shows the distal end of torquer head 630 as it approaches the central spinner assembly 624. Torquer head 630 may be engaged with implant 610 by pushing the proximal end of the torquer tube (not shown) in a distal direction while pulling the proximal end of central anchor lead 622 (shown in FIG. 23) in a proximal direction. The torquer tube may need to be rotated until tabs 678 engage with slots 636. FIG. 41 shows the distal end of torquer head 630 and central spinner assembly 624 with portions cut away to show further details of these components.

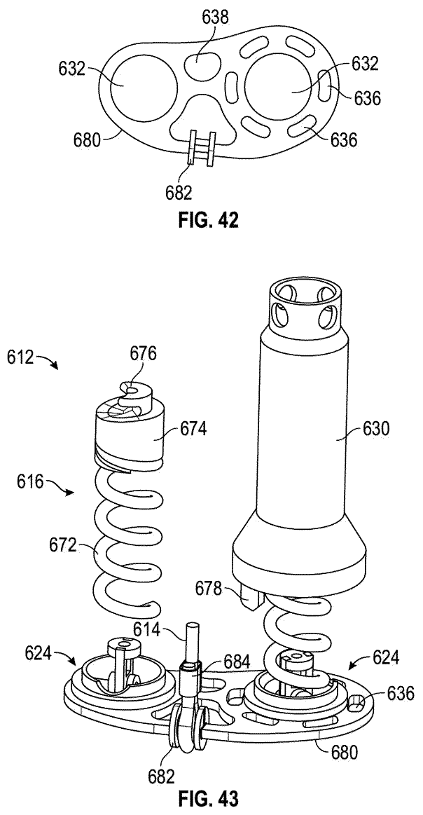

Referring to FIGS. 42 and 43, views of anterior implant 612 components are shown. FIG. 42 shows a bare anterior implant base plate 680 with only a tether thimble 682 attached. Since the two anterior implants 612 shown in FIGS. 23 and 24 are mirror images of one another, the same plate 680 and thimble 682 may be used to construct either one, depending on which direction spinner assemblies 624 are facing. When spinner assemblies 624 are mounted on the near side of plate 680 shown in FIG. 42 (such that the lead nuts 666 are pointed up, as shown in FIG. 43), a lateral anterior implant 612 is formed (shown on the left side of FIGS. 23 and 24.) When spinner assemblies 624 are mounted on the far side of plate 680 shown in FIG. 42 (such that the lead nuts 666 are pointed down, opposite of what is shown in FIG. 43), a medial anterior implant 612 is formed (shown on the right side of FIGS. 23 and 24.)

In this exemplary embodiment, identical components such the spinner assemblies 624, anchors 616, torque head 630, etc. are used for anterior implants 612 previously described for posterior implant 610. As shown in FIG. 42, the spacing of slots 636 may be the same as that used on the posterior implant plate 626 (shown in FIG. 25) for receiving the two opposing tabs 678 of torque head 630 (only one tab 678 seen in FIG. 43.) With slots 636 spaced every 60 degrees, torque head can be dithered no more than plus or minus 30 degrees or rotated no more than 60 degrees in one direction, before tabs 678 engage with a pair of mating slots 636.

As shown in FIG. 43, thimble 682 and sleeve 684 can be used to terminate the distal end of a tether 614 on a beam of an anterior implant 612 such that the tether can freely pivot relative to the implant. This can allow the implants 612 with their pre-attached tethers 614 to be more reliably loaded into and deployed from a delivery catheter. This pivoting also allows the tethers 614 to align directly towards the posterior implant 610 (as shown in FIGS. 23 and 24) rather than imparting rotational moments to the implanted anterior implants 612 and the underlying heart tissue.

In some embodiments, tethers or tensile members 614 have a composite structure. A continuous braided filament core may comprise an Ultra High Mechanical Polyethylene (UHMPE) fiber such as Dyneema® provided by Koninklijke DSM N.V. of the Netherlands, combined with a polyethylene terephthalate (PET) fiber. Dyneema® may be used for strength and durability and PET provides improved bonding with epoxy. In some embodiments, a 50%/50% combination of Dyneema® and PET is used. This continuous braided filament core may be inserted into or coated with a polyvinylidene fluoride (PVDF) jacketing to provide desirable handling characteristics, such as high column strength for threading the tether through a catheter and advancing the catheter without the tether collapsing. In some embodiments, at least one platinum wire is placed in the distal section of each tether 614 for radiopacity so that the tethers can be better seen under imaging. The filament core may be saturated with epoxy prior to being inserted into an outer jacket. This may be done to bind the composite together. In some embodiments, the outer jacket is run through a necking die to reduce its diameter and compress it into the filament. Tethers 614 may be color-coded so that the surgeons can distinguish a medial tether from a lateral tether. In some embodiments, markings are provided on the tethers 614 every 5 mm so that cinching may be observed. In some embodiments, tethers 614 have an outer diameter of 0.026 inches. Applicants have discovered that the use of the above features provides the ability to cut the tethers in vivo, provides tethers with superior longitudinal stiffness for responsive cinching and superior durability for the life of the implants while supporting the full in vivo load of heart valve adjustment.

Referring to FIGS. 44-46, views of the exemplary implants are shown preloaded into delivery systems. In some embodiments, the implants are each loaded into their own implant loader 686. Implant loader 686 has a proximal hub 688 (shown in FIG. 45) configured to slide over the distal end of an inner steerable catheter (not shown.) The distal end 690 of implant loader 686 may be configured to slide within the proximal end of an outer steerable catheter (not shown.) During deployment of the implant, the implant loader 686 remains in place in the proximal end of the outer catheter while the implant and distal end of inner catheter slide distally through implant loader 686 and the outer catheter.

FIG. 44 shows a posterior implant 610 preloaded into an implant loader 686. As shown, implant 610 is close to parallel with the central axis of implant loader 686. Five anchor leads 622 are each attached to a spinner assembly 624 on the implant and lie generally flat against implant 610 when preloaded. Each anchor lead 622 extends proximally through an anchor 616 and attached anchor driver 628. Only four anchors 616 are visible, as the fifth anchor is located inside torque head 630.

A pair of tether pullers 694 may each be threaded through a spinning eyelet assembly 620 of posterior implant 610 and extend distally out of the implant loader 686 as shown. After the anterior implants are implanted, their tethers 614 may be attached to the protruding ends of the tether pullers 694, such as with a sleeve attached to each puller that can be crimped onto the tether. The tethers may then be pulled proximally with the tether pullers 694 until the proximal ends of the tethers emerge from the inner steerable catheter (not shown.)

FIG. 45 shows a medial anterior implant 612 preloaded into an implant loader 686. As shown, implant 612 forms an acute angle with the central axis of implant loader 686. Two anchor leads 622 are each attached to a spinner assembly 624 on the implant and extend proximally through an anchor 616 and attached anchor driver 628. Only one anchor 616 is visible, as the second anchor is located inside the torque head 630 torque tube 692.

FIG. 46 shows a lateral anterior implant 612 that has been preloaded into an implant loader 686 and is being pushed out of its distal end 690. In this exemplary embodiment, the preloading and deployment of the lateral anterior implant 612 is essentially the same as that of the medial anterior implant 612 shown in FIG. 45, but a tether puller 694 is provided for attaching to the proximal end of the tether from medial anterior implant 612 which is implanted first. In this exemplary embodiment, an axially extending spring lumen 696 is provided to guide tether puller 694. In this embodiment, spring lumen 696 resembles an automobile “curb feeler” in that it may be deflected from a straight orientation by a lateral force in operation but is biased to return to its original straight orientation. Spring lumen 696 serves to prevent tether puller 694 and later a tether itself from wrapping about the implant, another tether, lead or tube.

Referring to FIGS. 47A, 47B and 24, an exemplary method of performing an annuloplasty procedure according to aspects of the present disclosure is schematically shown. The steps of this exemplary method 710 are similar to those previously described in reference to the flowchart shown in FIG. 4 and the series of images shown in FIGS. 5-22. For ease of understanding, descriptions of details that are the same between the two methods will not be repeated below. In some implementations of the method, two anterior implants 612 and one posterior implant 610 are implanted. In other implementations, different types or numbers of devices may be used.

In some implementations of method 710, the first step 712 of the method is introducing the distal end of a steerable outer delivery catheter (not shown) into the left atrium of a subject. This may be performed using a transseptal approach, a left atrial approach or other methodology for gaining access to the left atrium. In some implementations, an inner dilator (not shown) is located in the distal end of the steerable outer delivery catheter for crossing the septum. A steerable inner delivery catheter (not shown) may be placed through the outer steerable delivery catheter for more precise delivery of the implants.

In step 714 of this exemplary embodiment, once the inner delivery catheter is introduced into the outer catheter, a first anterior implant 612 (see FIG. 24), sometimes referred to herein as a first member, and the distal end of the inner delivery catheter may be deployed into the left atrium from the distal end of the outer delivery catheter in. In this exemplary embodiment, a medial anterior device 612 is implanted first, followed by a lateral anterior device 612. In other embodiments, the order of implantation may be changed. Anchor leads 622 and the inner catheter may be used to push anterior implant 612 through the outer delivery catheter and deploy it from the distal end. After the first anterior implant 612 emerges from the distal end of the outer delivery catheter, anchor leads 622 may be manipulated from the proximal end of the inner delivery catheter to pivot anterior implant 612 into an orientation that is generally perpendicular to the inner delivery catheter. The first anterior implant 612 emerges from the outer delivery catheter with the distal end of its tether or first tensile member 614 pre-attached. The proximal end of the attached tether 614 extends through the delivery catheters and out the proximal ends. Torque head 630 may be slid distally over anchor lead 622 until distally extending tabs 678 fit into slots 636 (see FIG. 43) so that implant 612 may be steered into its desired implantation location and orientation. Alternatively, torque head 630 may remain stationary relative to the catheter and anchor lead 622 may be used to pull implant 612 proximally until it engages with torque head 630.

In step 716, anterior implant 612 (i.e., the first member) is anchored to the anterior side of the mitral valve. This may be accomplished by individually turning each of the two helical tissue anchors 616 with their attached driver heads 628. While torque head 630 holds anterior implant 612 against the mitral valve annulus tissue, one drive tube may be rotated to screw its anchor 616 through its spinner assembly 624 and into the underlying tissue. The torque head 630 may be used to then finely adjust/rotate implant 612 before the second anchor 616 is screwed into place with its drive tube and driver head 628. Proper placement of the first member may be confirmed through imaging. When the surgical staff is ready to remove the delivery instrumentation, leads 622 may be unscrewed from spinner assemblies 624, withdrawn past driver heads 628 and at least partially into the connected drive tubes. This allows the driver heads 628 to disengage from the anchors 616. Once driver heads 628 are disengaged, the attached drive tubes, anchor leads 622, torque head 630 and inner catheter can be proximally withdrawn through the outer delivery catheter.

In this exemplary embodiment, steps 718 and 720 are similar to steps 714 and 716, respectively. In step 718, a lateral anterior implant 612 (sometimes referred to herein as a second member) and the distal end of an inner delivery catheter may be deployed into the left atrium from the distal end of the outer delivery catheter in much the same way as previously described for the medial anterior implant 612 in step 714. In some embodiments, a separate, pre-loaded and pre-sterilized steerable inner catheter is provided for each of the anterior implants 612. In some embodiments, the tether 614 from the previously implanted first member 612 remains in the outer catheter after the inner catheter for the first member has been removed. To avoid entanglement, this tether 614 may be threaded through the second inner catheter before the second inner catheter is introduced into the outer steerable catheter. In this exemplary embodiment, the second member 612 is deployed with its own tether or tensile member 614 attached, such that the proximal ends of both the first and second tethers 614 extend through the second inner catheter and out of its proximal end. In step 720, the lateral anterior implant 612 (i.e., the second member) is anchored to the anterior side of the valve in much the same way as previously described for the medial anterior implant 612 (i.e., the first member.)

In step 722, posterior implant 610 (sometimes referred to herein as a third member) is deployed into the heart. As with the first and second members, the third member may be provided to the surgeons preloaded into its own steerable inner catheter. In some embodiments, before the third inner catheter is introduced into the outer catheter, the tethers or tensile members 614 extending from the first and second members are threaded through eyelet assemblies 620 on posterior implant 610 and through the third inner catheter. Anchor leads 622 and the inner catheter may be used to push anterior implant 612 through the outer delivery catheter and deploy it from the distal end. After the posterior implant 610 emerges from the distal end of the outer delivery catheter, anchor leads 622 may be manipulated from the proximal end of the inner delivery catheter to pivot posterior implant 610 into an orientation that is generally perpendicular to the inner delivery catheter. By keeping some tension on the proximal ends of the tethers 614 connected to the implanted first and second members, the posterior implant 610 or third member emerges from the outer delivery catheter and tracks over the first and second tensile members 614. One advantage to this arrangement is that the first and second tensile members 614 help guide the third member 610 into a proper orientation. Having the tensile members 614 pre-connected to the three implants also saves time during the surgical procedure and ensures that the tensile members are properly and consistently connected to the implants. Torque head 630 may be slid distally over the central anchor lead 622 until distally extending tabs 678 fit into slots 636 (see FIG. 40) so that implant 610 may be further steered into its desired implantation location and orientation. Alternatively, torque head 630 may remain stationary relative to the catheter and anchor lead 622 may be used to pull implant 610 proximally until it engages with torque head 630.

In step 724, posterior implant 610 (i.e., the third member) is anchored to the posterior side of the mitral valve. This may be accomplished by individually turning each of the five helical tissue anchors 616 with their attached driver heads 628. While torque head 630 holds posterior implant 610 against the mitral valve annulus tissue, one drive tube may be rotated to screw its anchor 616 through its spinner assembly 624 and into the underlying tissue. The torque head 630 may be used to then finely adjust/rotate implant 610 before the next anchors 616 are screwed into place with their drive tubes and driver heads 628. Proper placement of the third member may be confirmed through imaging. When the surgical staff is ready to remove the delivery instrumentation, leads 622 may be unscrewed from spinner assemblies 624, withdrawn past driver heads 628 and at least partially into the connected drive tubes. This allows the driver heads 628 to disengage from the anchors 616. Once driver heads 628 are disengaged, the attached drive tubes, anchor leads 622, torque head 630 and inner catheter can be proximally withdrawn through the outer delivery catheter.

In other embodiments, the order of deployment and attachment of the multiple implants can be changed. In these other embodiments, the first implant or implants are deployed with tether(s) attached, and at least one subsequently deployed implant tracks over the tether(s) when it is being deployed and attached to heart tissue. For example, a posterior implant may be implanted first with two tethers pre-attached at opposite ends of the implant. A medial anterior implant may then be deployed, tracking over one of the tethers of the posterior implant. After the medial anterior implant is secured to underlying heart tissue, a lateral anterior implant may be deployed, tracking over the other tether of the posterior implant. In another embodiment, a single anterior implant with two tethers is implanted first, and a single posterior implant may then be deployed, tracking over the two tethers of the anterior implant. Other embodiments having different orders of implant deployment may also utilize the principles of the present disclosure.

In exemplary method 710, once all three implants have been deployed and anchored, additional tension may be applied to the interconnecting tethers 614 to draw the anterior and posterior sides of the mitral valve into closer approximation. This may be accomplished in steps 726, 728 and 730 of method 710. In step 726, a first lock 618 is deployed over the first tensile member 614 which is connected to the medial anterior implant 612. In step 728, a second lock 618 is deployed over the second tensile member 614 which is connected to the lateral anterior implant 612. The locks 618 may be pushed over the tensile members 614 from their proximal ends by sleeve-like tools (not shown) that urge the locks 618 distally until they abut against eyelet assemblies 620, as shown in FIGS. 23 and 24. In step 730, tension is then applied to the first and second tensile members 614 by pulling proximally on the tensile members while pushing distally against locks 618 with the sleeve-like tools.

In some implementations, tension in tensile members 614 may be increased simultaneously. In some implementations, tension may be increased incrementally in members 614, alternating between the two until the desired tensions and or valve approximation is reached. In some implementations, the final tension and or tissue approximation of each tensile member 614 is approximately the same. In some implementations, the final tension and or tissue approximation of each tensile member 614 is different. Because medial and lateral cinching can be performed independently, the placement of each implant is more forgiving. In some implementations, real time echocardiography of the mitral valve is used to monitor the reduction in mitral regurgitation as tensile members 614 are tightened. In some embodiments, one or both locks 618 may be temporarily released if it is desired to reduce the tension in the tensile members 614.

After the desired tensions and or tissue approximations are obtained, the excess length of tensile members 614 extending proximally from locks 618 may be cut off. In step 732, a cutter assembly may be slid distally along each tensile member 614 until it reaches the lock 618. It may then be activated to cut the tensile member and then withdrawn with the cut off portion of the tensile member. In step 734, the outer delivery catheter may then be withdrawn from the left atrium.

In some embodiments, the systems and methods disclosed herein, or portions thereof may be utilized in a similar manner on either atrioventricular valve.

Advantages provided by the systems and methods disclosed herein can include the following. A more direct reduction in the anterior-posterior (A-P) direction can be achieved. Because the A-P direction is the most clinically relevant dimension to be reduced, it is advantageous to directly affect this dimension as opposed to simultaneously changing other dimensions of the annulus. This can be accomplished with a reduced cinching force because the action is directly in the A-P direction, rather than larger forces that are generally needed with circumferential remodeling. Lower cinching force typically translates to fewer anchors required. The systems and methods also allow for a high level of customization to suit a particular anatomy. This relates to there being distinct components that are placed separately, and the ability to adjust the medial and lateral sides separately. The separate components are each easier to implant compared with one superstructure. Each of the components can be retrieved prior to anchor detachment. The systems and methods allow for in vivo adjustability, allow for reduced accuracy needed to place the components and simplify the implantation procedure. A smaller number of implant sizes and configurations can also be accommodated. In addition to tensioning the device during the de novo procedure, additional tensioning devices can be added at a later time or date, and or the existing devices can be re-tensioned to further reduce the A-P dimension.

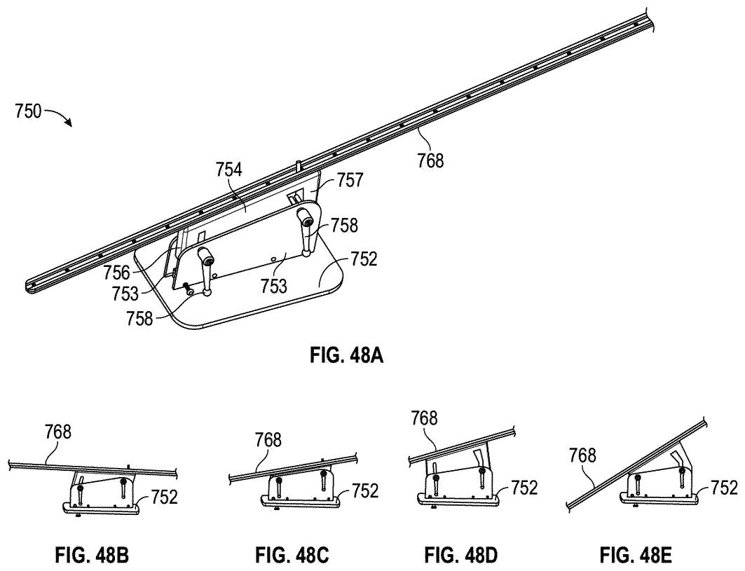

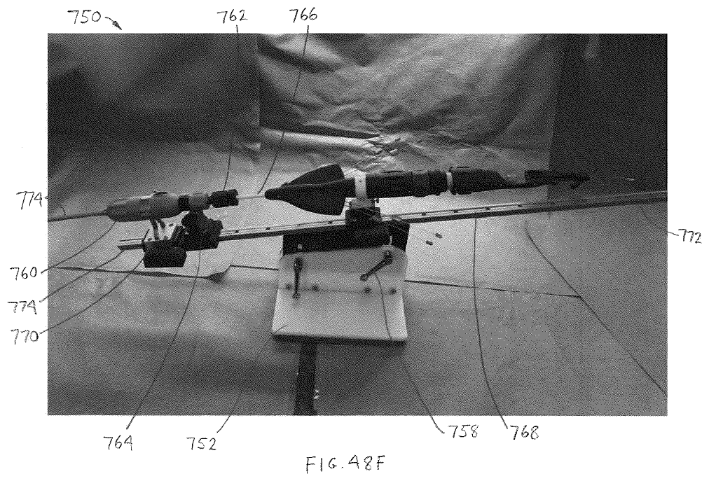

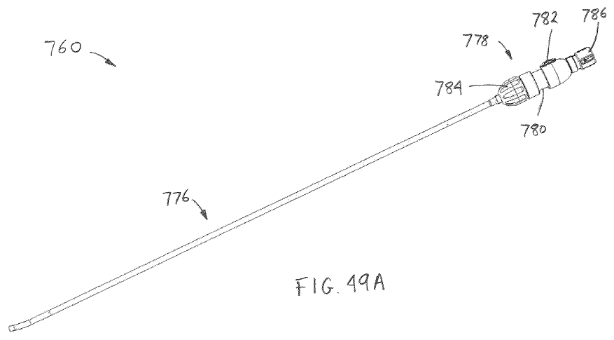

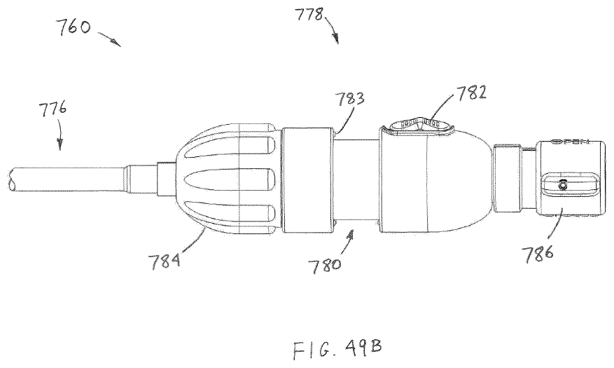

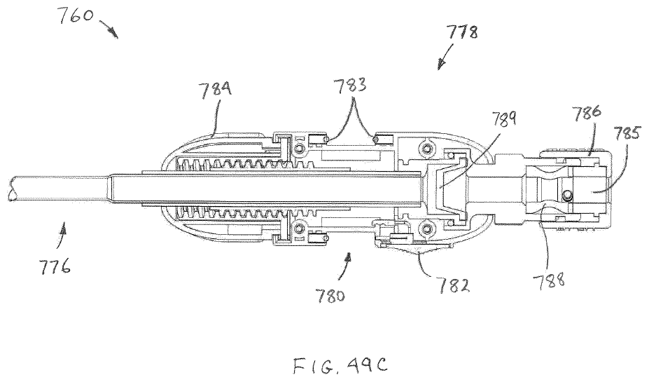

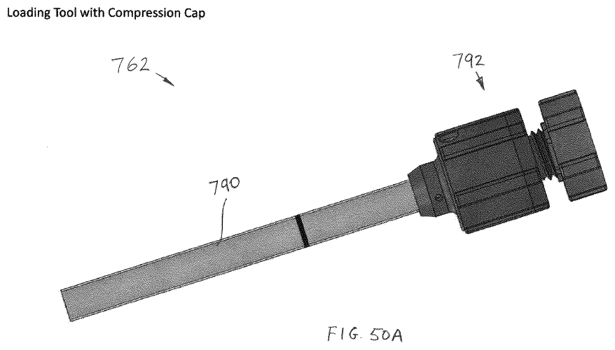

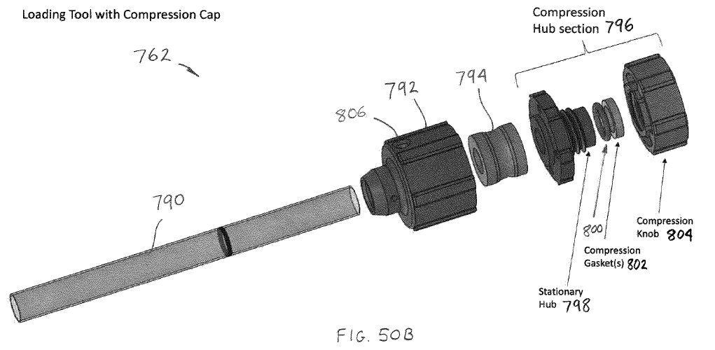

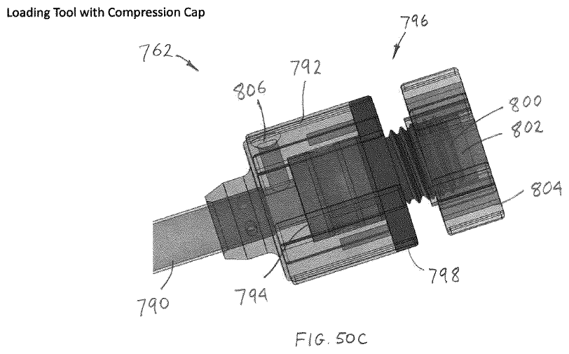

Referring to FIGS. 48A-62F, details of exemplary surgical instrumentations that may be constructed and used to implant the previously described devices according to aspects of the present disclosure are provided.

Referring first to FIG. 48A, an exemplary adjustable base system is provided for slidably supporting the proximal ends of some or all of the above-mentioned instrumentations. The exemplary system includes a weighted base 752 configured to rest upon an operating table, cart, stool or other platform (not shown) adjacent to a patient being operated upon. Clamps (not shown) can be used to hold base 752 to the underlying platform, or base 752 can be fabricated with sufficient weight of its own to prevent it from moving during a surgical procedure. Base 752 can be provided with a pair of spaced apart vertical plates 753 extending upwardly from it as shown. Plates 753 may be configured to slidably receive an adjustable bracket 754 therebetween, with a linear guide rail 768 mounted to the top of bracket 754. Slots 756 and 757 may be provided through bracket 754 for receiving threaded shafts from clamp handles 758. With this arrangement, clamp handles 758 can be loosened, the height and or angle of rail 768 can be adjusted, and handles 758 re-tightened in order to releasably lock the orientation of rail 768 relative to base 752 and the patient.

Referring to FIGS. 48B-48E, the exemplary adjustable base system of FIG. 48A is shown in various orientations. In particular, FIG. 48B shows rail 768 in a lowered position with a slight negative angle (i.e., tilted downward away from the patient.) FIG. 48C shows rail 768 in a lowered position with a slight positive angle (i.e., tilted downward towards the patient.) FIG. 48D shows rail 768 in a raised position with a moderate positive angle. FIG. 48E shows rail 768 in a raised position with a steep positive angle. Many other orientations may be obtained with the base system. In some embodiments, the base system may be adjusted anywhere between a negative 10-degree angle and a positive 45 degree angle. In some embodiments, the range of height adjustment is at least 3 inches.

Referring to FIG. 48F, proximal ends of some of the previously mentioned instrumentations are shown as they may be configured for use during a surgical procedure. System 750 includes an outer steerable catheter assembly 760, implant loading tool assembly 762, tether retainer yoke 764 and inner steerable catheter assembly 766. The outer steerable catheter assembly 760, tether retainer 764 and inner steerable catheter assembly 766 may each be slidably attached to a linear guide rail 768 by a separate carriage assembly 770. In some implementations, guide rail 768 is a 30 mm wide Drylin® T Standard guide rail, and carriage assemblies 770 are Drylin® T Standard recirculating ball bearing carriages, both manufactured by Igus, GmbH of Cologne, Germany. In another embodiment the carriage assembles have an inner profile that matches the profile of the guide rail. In other embodiments the carriages snap over the guide rail at any point along its length without the need to slide the carriage over the end of the rail. Carriage assemblies 770 may be provided with locking knobs that may be moved between a locked state in which the carriage is fixed in its current position on rail 768, and a free state in which the carriage may be moved along the rail. In other embodiments, means of applying specific friction (such as ball indents) can be used to achieve a desired friction on the linear guide to allow smooth linear motion but prevent inadvertent sliding. In some implementations, guide rail 768 is 60-190 cm long and inclined at an angle of 0 to 45 degrees above horizontal. In some implementations, guide rail is 120 cm long with a 15 degree angle. In the exemplary embodiment shown in FIG. 48F, linear guide rail 768 includes an upper proximal end 772 and a lower distal end 774.

Referring to FIGS. 49A-49C, outer steerable catheter assembly 760 is shown. Catheter assembly 760 includes a catheter section 776 (which is subsequently described in further detail) and a handle assembly 778. Handle assembly 778 includes a mounting groove 780 for removably attaching handle 778 to a rail carriage assembly 770, as shown in FIGS. 48A-48C. Handle 778 may be rotatably attached so that the surgeon can rotate outer catheter assembly 760 relative to the rail carriage assembly. This allows the surgeon to rotationally orient the distal end (not shown) of catheter section 776 within the patient's circulatory system and heart. In this exemplary embodiment, a single pull cable extends between a pull ring at the distal end of catheter section 776 and handle assembly 778 for pulling the distal end from a generally straight orientation into a curved orientation. Rotation lock 782 may be provided on handle assembly 778 as shown to indicate to the surgeon the radial/rotational orientation of outer catheter 760 and to releasably lock the orientation in place. Ball detents 783 may be provided as shown in FIG. 49C to provide tactile feedback when handle assembly 778 is being rotated, and to provide frictional engagement to keep the handle in its current rotational orientation. Steering knob 784 is provided on the distal end of handle assembly 778 and is coupled to an internal lead screw mechanism as shown in FIG. 49C for pulling on the proximal end of the pull cable. With this arrangement, the more steering knob 784 is twisted, the more the distal end of the outer catheter 776 is curved for navigating within the patient.

An internal bore 785 may be configured to receive the distal end of implant loading tool assembly 762, as will be subsequently described. A cam-action collar seal 786 may be provided on the proximal end of handle assembly 778 for tightening down an hourglass-shaped valve 788 (shown in FIG. 49C) by compressing it longitudinally. In this exemplary embodiment, collar 786 and valve 788 provide a hemostasis seal configured to minimize blood loss during the surgical procedure. Collar 786 may be used to completely close valve 788 when just the tethers are passing through the handle. In this exemplary embodiment, collar 786 is twisted 180 degrees when going from the open position to the closed position, and the words “OPEN” and “CLOSED” are molded into opposite sides of collar 786. A cross-slit valve 789 may also be provided as shown in FIG. 49C to allow a loading tool to be rapidly inserted or withdrawn from handle assembly 778 with minimal blood loss. Handle assembly 778 may also be provided with a flush port (not shown.)

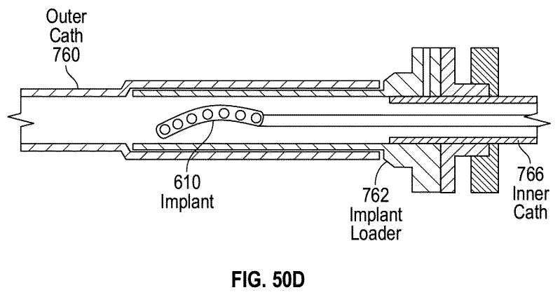

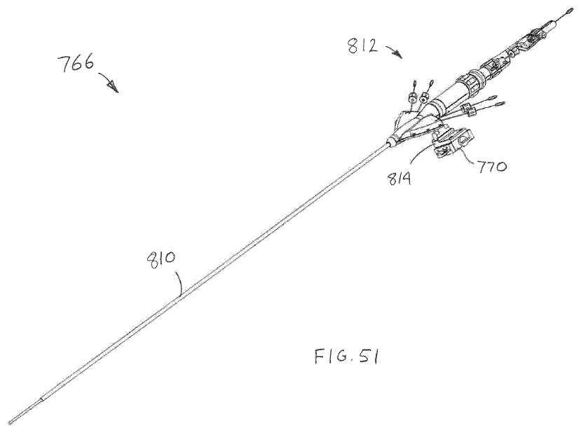

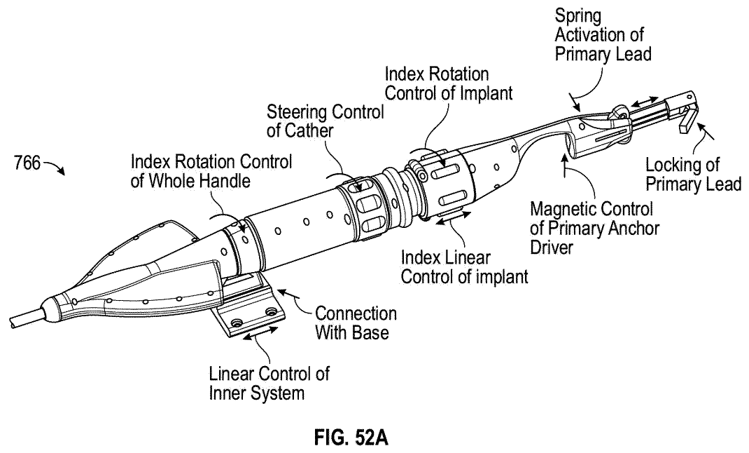

Referring to FIGS. 50A-50C, three views of an exemplary implant loading tool assembly 762 are provided, similar to the implant loaders shown in FIGS. 44-46. FIG. 50A is a perspective view showing tool 762 assembled, FIG. 50B is an exploded view showing the components of tool 762, and FIG. 50C is an enlarged transparent view of the proximal end of tool 762 showing internal features. As best seen in FIG. 50B, loading tool 762 includes a rigid cannula 790, a main housing 792 attached to the proximal end of cannula 790, a hemostasis seal 794 located inside housing 792 when assembled, and a compression hub section 796 that seals the proximal end of housing 792. Compression hub section 796 includes a stationary hub 798, compression gaskets 800 and 802, and a compression knob 804. Three screws (not shown) may be used to attach stationary hub 798 to main housing 792.

The cannula 790 of implant loading tool assembly 762 is configured to be received within the proximal end of outer catheter 760, and tool 762 is configured to allow inner a steerable catheter assembly 766 to pass through it and into outer catheter 760. In some implementations, a separate inner steerable catheter assembly and loading tool assembly 762 are used to introduce each device that is implanted in the patient's body. Compression gaskets 800 and 802 serve to seal around the outside of an inner catheter assembly or other instrumentation placed through loading tool 762. Compression knob 804 may initially be tightened to compress gaskets 800 and 802 to make a good seal and inhibit an inner catheter from rotating relative to loading tool 762. After loading tool 762 has been loaded into the proximal handle of the outer catheter, compression knob 804 may be loosened to allow the inner catheter to rotate and easily slide in and out of the outer catheter. A flush port 806 may also be provided in main housing 792 as shown.

Referring to FIG. 50D, a longitudinal cross-section schematically shows implant loading tool assembly 762 in use when placed in the proximal end of outer steerable catheter 760. The handle assembly of outer catheter 778 is omitted in FIG. 50D for clarity. Loading tool 762 is shown attached to the distal end of an inner steerable catheter 766 with compression knob 804 tightened, and with previously described posterior implant 610 preloaded in tool 762. The proximal end of outer steerable catheter 760 may be enlarged as shown so that when loading tool 762 is placed therein, the inner diameter of tool 762 is substantially the same as the inner diameter of the main portion of outer catheter 760. A separate loading tool 762 may be provided with each inner steerable catheter 766. Each tool 762 may come packaged attached to the distal end of the inner catheter assembly with an implant preloaded into the tool. This arrangement allows for an easy exchange of inner catheters and implants within outer catheter 760 but does not require that the implant fit inside the inner catheter assembly. The implant need only fit within the inside diameter of the main portion of outer catheter assembly 760, through which the inner catheter assembly 766 also slides.

In operation, implant loading tool assembly 762 may come packaged releasably attached onto the distal end of inner catheter 766 with compression hub 796. Compression hub 796 keeps tool 762 attached to inner catheter assembly 766 and prevents it from rotating relative to inner catheter assembly 766 so that implant 610 and its leads do not become twisted. After unpackaging, loading tool 762 and the distal end of inner catheter 766 may be inserted into the proximal end of outer catheter 760. Compression hub 796 may then be loosened, allowing inner catheter 766 and implant 610 to be slid distally through outer catheter 760.

Referring to FIGS. 51-56, an exemplary steerable inner catheter 766 is shown. Inner catheter 766 may be used to deliver and implant a posterior implant 610 (shown in FIGS. 23 and 24.) As best seen in FIG. 51, inner catheter 766 includes a catheter section 810 and a control housing 812 mounted on the proximal end of the catheter section 810. Control housing 812 may be rotationally coupled to a support structure 814 which in turn may be mounted to a guide rail carriage 770, as previously described. Ball detents or another detent mechanism may be provided as previously described relative to the outer catheter assembly to provide tactile feedback such as periodic resistance when the inner catheter control housing 812 is being rotated, and to provide frictional engagement to keep housing 812 in its current rotational orientation.

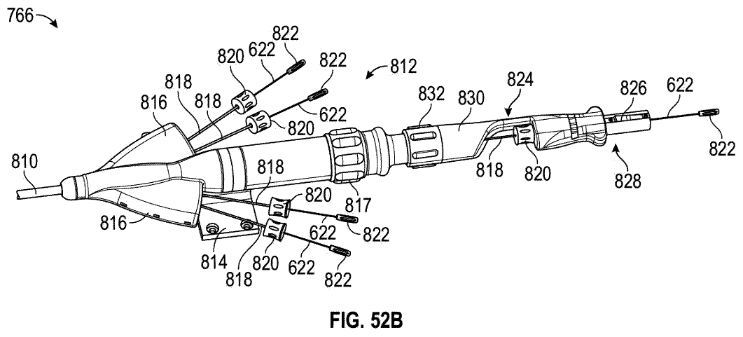

As best seen in FIG. 52B, a distal portion of housing 812 may be provided with a pair of opposing, radially extending wings 816. Wings 816, among other functions, serve as an indicator as to the orientation of the distal tip of catheter section 810 as it is being maneuvered within the patient's heart. Control housing 812 may be rotated during a surgical procedure relative to support structure 814 to change its radial orientation and that of the distal tip of catheter section 810. Steering knob 817 may be twisted by the surgeon to increase or decrease the amount of curvature of the distal tip. In this exemplary embodiment, steering knob 817 drives an internal sled through a lead screw assembly (not shown), which in turn drives a pair of steering cables (not shown) that extend along the length of catheter section 810 to its distal tip. One steering cable pulls on one side of the distal tip while the other cable provides slack on the opposite side, thereby allowing the distal tip to curve in a first direction. When the steering knob 817 is turned in the opposite direction past its original neutral position, the other cable pulls on the opposite side of the distal tip, thereby curving the distal tip in the opposite direction.

One of the wings 816 may be labelled with an M for “medial” and the other labeled with an L for “lateral”, as shown. The medial wing 816 may be provided with a pair of internal guide slots (not shown) that guide the proximal portions of two medial anchor driver tubes 818 outwardly from catheter section 810 to a location proximal to medial wing 816 where they can be operated by a surgeon. The proximal ends of medial anchor driver tubes 818 may be provided with control knobs 820 for rotating medial driver tubes 818, which in turn drive medial anchor driver heads 628, shown on the right in FIG. 24, at the distal ends of anchor driver tubes, as previously described. The proximal ends of medial anchor leads 622 are shown extending from the proximal ends of medial driver tubes 818 in FIG. 52B. Smaller diameter control knobs 822 may be provided on the proximal ends of medial anchor leads 622 for rotating the leads, which in turn are connected at their distal ends to the two medial spinner assemblies 624, shown on the right in FIG. 24 and as previously described.

In a similar fashion to the medial wing 816, lateral wing 816 may be provided with a pair of internal guide slots (not shown) that guide the proximal portions of two lateral anchor driver tubes 818 outwardly from catheter section 810 to a location proximal to lateral wing 816 where they can be operated by a surgeon. The proximal ends of lateral anchor driver tubes 818 may be provided with control knobs 820 for rotating lateral driver tubes 818, which in turn drive lateral anchor driver heads 628, shown on the left in FIG. 24, at the distal ends of anchor driver tubes, as previously described. The proximal ends of lateral anchor leads 622 are shown extending from the proximal ends of lateral driver tubes 818 in FIG. 52B. Smaller diameter control knobs 822 may be provided on the proximal ends of lateral anchor leads 622 for rotating the leads, which in turn are connected at their distal ends to the two lateral spinner assemblies 624, shown on the left in FIG. 24 and as previously described.

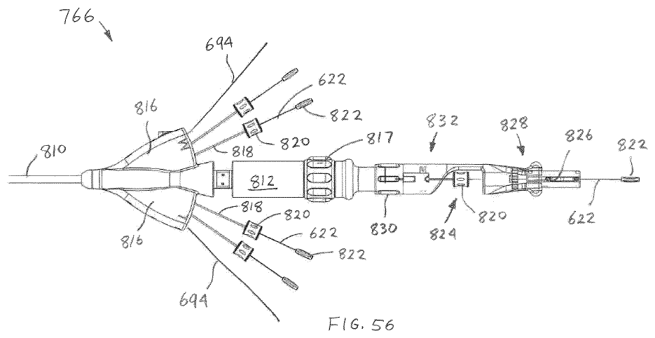

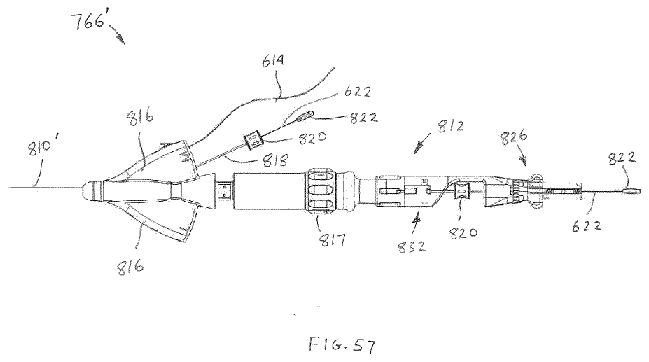

The two wings 816 together form a manifold that present drive tubes 818, leads 622 and tethers or tether pullers 694 (see FIG. 56) to the surgeon in a flat, fan shape. The linear arrangement of the drivers and tethers provides an intuitive relationship between the driver on the proximal end and with their arrangement on the implant.

A central anchor driver tube 818 may extend through the center of housing 812 and terminate with a control knob 820 in a recessed portion 824 of housing 812. This driver tube 818 may be used for rotating the central anchor driver head 628, shown in the center of FIG. 24, as previously described. In this exemplary embodiment, a first magnet is fixed inside the center anchor control knob 820 and a mating second magnet is fixed inside the proximal portion of control housing 812 to prevent the central anchor driver 818 from inadvertently advancing during the procedure. The proximal end of central anchor lead 622 is shown extending from the proximal end of control housing 812 in FIG. 52B. A smaller diameter control knob 822 may be provided on the proximal end of central anchor lead 622 for rotating the lead, which in turn is connected at its distal end (FIG. 24) to a central spinner assembly 624 (not shown.) A lock lever 826 may be provided as shown in FIG. 52B to releasably couple central lead 622 to the handle to prevent relative motion with the handle and therefore prevent relative motion between the implant and the handle. In this exemplary embodiment, lever lock 826 is coupled to a spring actuation mechanism 828. Spring actuation mechanism 828 serves to bias central lead 622 proximally with a resilient spring force when lever 826 is engaged. This arrangement helps retain implant 610 against torquer head 630 (shown in FIG. 24) when manipulating implant 610 with inner steerable catheter control housing 812 (shown in FIG. 52B.) In some embodiments the central anchor can be eliminated, relying on the other anchors to provide fixation. The torquer would not need to operate around a central anchor and lead nut would not need to mount on a spinner assembly. In such embodiments, the diameter of the torquer could be reduced allowing for easier loading. An inside diameter of the torquer could be smaller to more closely match a diameter of a lead. The tighter diameter alignment between the inner diameter of the torquer and lead would reduce side to side movement and allow a torquer to engage with the posterior implant with less manipulation.

Control housing 812 may also be provided with a torquer control assembly 830. The housing for torquer control assembly 830 is generally C-shaped and forms the previously described recessed portion 824. A torquer control knob 832 may be formed into the housing for torquer control assembly 830 that allows a surgeon to rotate and axially translate the entire housing for the torquer control assembly 830 relative to the rest of the control housing 812. Internally, torquer control knob/housing 832 is connected to the proximal end of torque tube 692 (not shown in FIG. 52B), the distal end of which is connected to torque head 630 as shown in FIG. 45. (Torque head 630 itself is best seen in FIGS. 24 and 37.)

In operation, the proximal end of central lead 622 may be pulled proximally after the implant has been deployed into the left atrium to pull the implant against torque head 630 (i.e., from a configuration in which implant 610 is separated from torque head 630 as shown in FIG. 37 to a configuration in which implant 610 is engaged with torque head 630 as shown in FIG. 24.) Torquer control knob/housing 832 may need to be rotated slightly to ensure the tabs of torque head 630 engage with the slots of the implant plate, as previously described. Once torque head 630 is fully engaged with the implant, spring actuation mechanism 828 may be depressed distally and lever 826 engaged to keep central lead 622 biased proximally, keeping torque head 630 engaged with the implant. The implant can now be rotated and translated with knob 832 to finely position the implant within the patient. Because torquer control assembly 830 extends around recessed portion 820 and supports spring actuation mechanism 828, central lead 622 will rotate and translate in unison with the torque head and the implant. This arrangement keeps the torque head against the implant when the implant is being translated by torquer control assembly 830, and keeps central lead 622 from being unscrewed from the implant when the implant is being rotated by torquer control assembly 830.

In some embodiments, torquer control assembly 830 is provided with a detent mechanism that provides periodic resistance when torquer control assembly 830 is used to rotate the torque tube and or when it is used to move the torque tube in an axial direction. What is meant by “periodic resistance” is a series of stops, detents or clicks, so that the surgeon receives feedback on how fast the control and implant are being moved, and so that the implant is held in place when the torquer control assembly 830 is not being moved. In some embodiments, the rotational connection of the inner catheter assembly to its carriage assembly detent mechanism has a greater rotational resistance than the torquer control knob assembly detent mechanism. This helps ensure that the inner catheter assembly is not inadvertently rotated when the torquer control assembly is being rotated. In this exemplary embodiment, torquer control assembly 830 is provided with 11 detent positions as knob 832 is moved linearly/axially over a stroke of 10 mm, and 36 detent positions as knob 832 is rotated one full revolution.

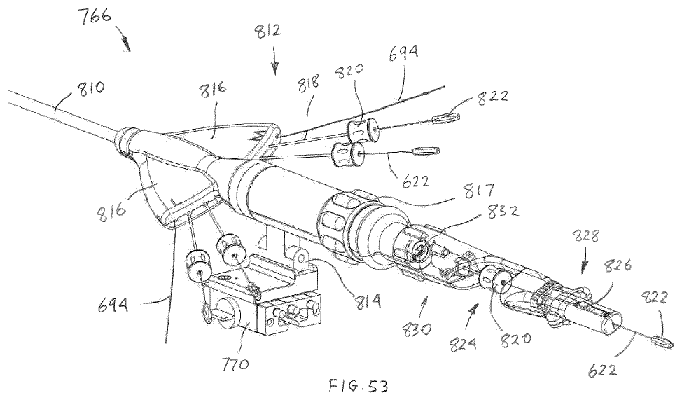

Referring to FIGS. 53-56, additional views showing the above-described features of inner catheter 766 are provided. As best seen in FIG. 54, control housing 812 may be rotated towards a surgeon positioned above and to the left of control housing 812 (in the direction of Arrow A) or away from the surgeon (in the direction of Arrow B.) FIG. 54 shows wings 816 moved 25 degrees towards a surgeon from an initial horizontal position. In this orientation, the distal tip of the inner catheter is moved in an anterior direction within the patient's heart. When implanting the posterior implant in this exemplary embodiment, the outer steerable catheter does most (and in some embodiments all) of the anterior and posterior positioning. When implanting the trigone implants, the inner steerable catheter is more “active” in the anterior and posterior as well as the medial and lateral positioning. In this exemplary embodiment, support structure 814 is configured to allow control housing 812 to be rotated plus or minus 90 degrees. In other embodiments, up to plus or minus 180 degrees of rotation are allowed. Preventing 360 rotation of the inner system prevents the physician from wrapping the tethers around each other in the anatomy and/or inside the outer guide. A detent mechanism may be provided that allows for 28 discrete positions in this 180-degree range of motion. As best seen in FIG. 55, control housing 812 may be provided with a flush port 833 having a tube located in a recessed groove spanning between a connection in the center of the bottom of the housing and a valve mounted on the bottom peripheral edge of medial wing 816. The support structure and rail carriage has been omitted from FIG. 56 for clarity.

Referring to FIG. 56, each wing 816 may be configured to support the proximal end of a tether puller 694 as shown. When supplied to the operation room, inner catheter assembly 766 may be provided with the two tether pullers 694, each extending through a wing 816, through inner catheter 810, through an eyelet assembly 620 in posterior implant 610 (both shown in FIG. 23) which is loaded into an implant loading tool 686 or 762 (as shown in FIG. 44) and extending out the distal end of the implant loading tool. The distal ends of the tether pullers may be provided with crimp sleeves or other attachment devices in order to grasp the proximal ends of the tethers 614 (distal ends of tethers 614 shown in FIGS. 23 and 24).

In some implementations, both anterior implants are implanted first and their tethers extend out the proximal end of the outer catheter, as will be subsequently described in more detail. The distal ends of the tether pullers can be different colors, different lengths, labeled with letters, and or have another identifying characteristic so that the medial and lateral tethers can be distinguished from each other at their distal ends. The proximal end of the tether from the medial implant can then be attached to the distal end of the medial tether puller 694, such as by inserting the tether into a crimp sleeve and crimping it. In other embodiments the interior of the crimp sleeve is provided with barbs facing in the proximal direction that allow the sleeve to grip the tether without the need of crimping. In addition, a peel-away-funnel may be provided to help guide the tether into the sleeve. The lateral tether may be attached to the lateral tether puller 694 in the same or a similar way. The two tethers may then be pulled through the implant and the inner catheter by the tether pullers 694 until they extend from the proximal side of the wings 816. They can then be cut or otherwise separated from the tether pullers 694. In some implementations, the inner catheter 766 is then inserted through the outer catheter while a light tension is applied on the tethers. In another embodiment the tethers can be fixated with respect to the linear guide. This would eliminate the need for an operator to apply light tension on the tethers. This can involve clamping the tethers directly to the linear guide or with an additional carriage with tether locking features. To further fine tune the tension and/or relative length of the tethers with respect to the system, a tether locking feature can rotate to either take up or provide more tether length. This arrangement enables inner catheter 766, an implant loader and a loaded implant to be provided in the operating room ready to insert into an outer catheter without the need to remove the implant and thread tethers through it first. This arrangement also inhibits the tethers from becoming crossed or tangled when the posterior implant is tracked over them during implantation. In other embodiments, the tethers are shorter, remain inside the lumens and do not exit the manifold until the inner system is mostly inserted into the outer guide. In these embodiments the tether pullers are used to maintain light tension during advancement. In other embodiments, the tethers are short enough that they do not exit the manifold even when the inner system is fully inserted.

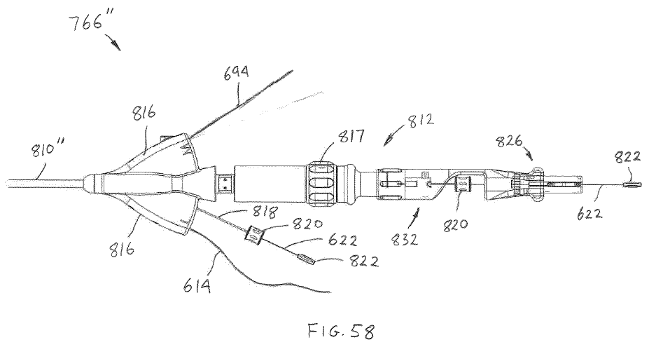

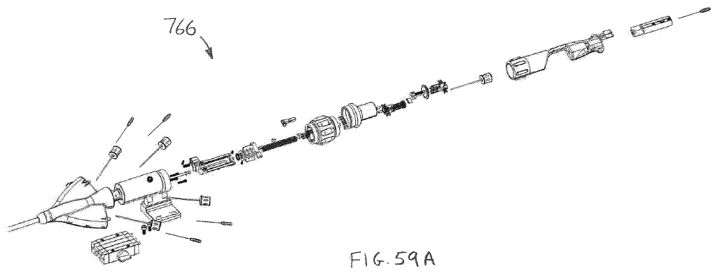

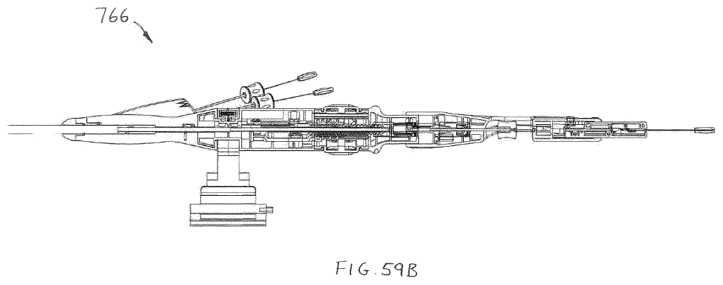

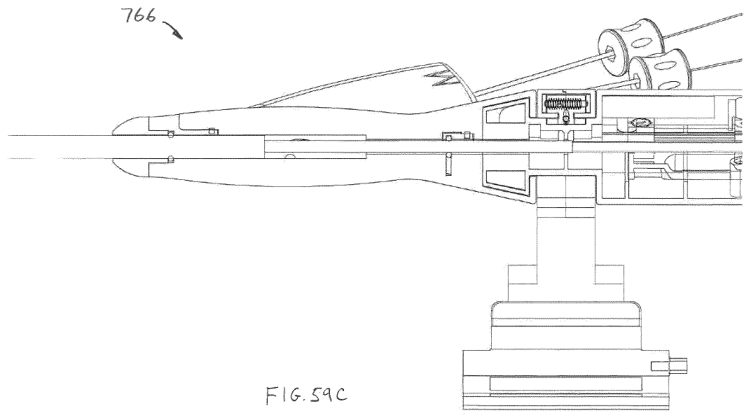

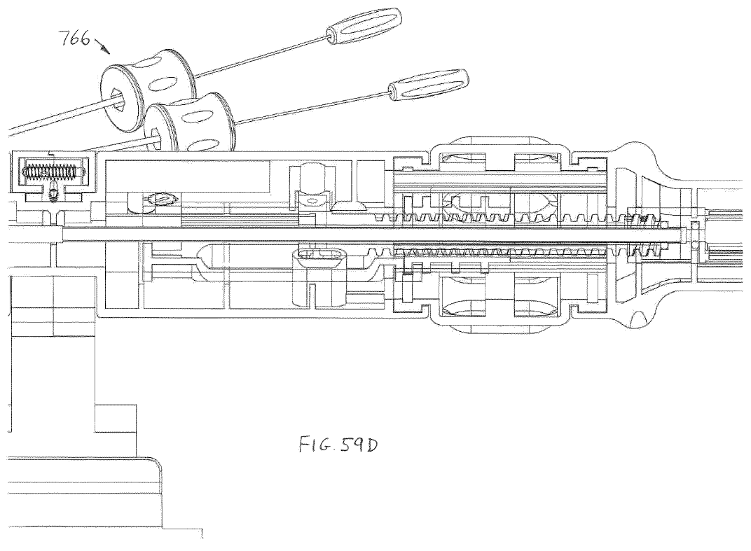

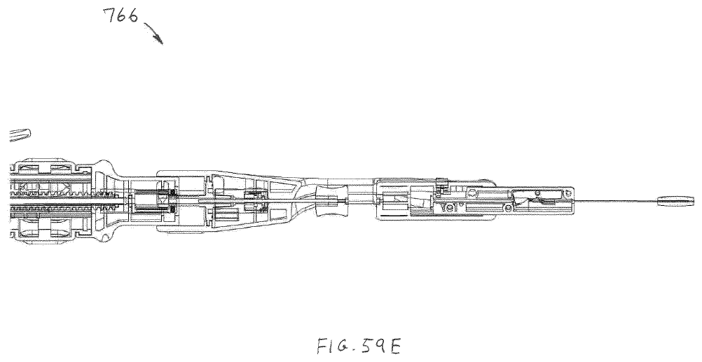

Referring to FIGS. 57 and 58, an exemplary inner catheter assembly 766′ configured for implanting a medial anterior implant 612 (shown in FIG. 23), and an exemplary inner catheter assembly 766″ configured for implanting a lateral anterior implant 612 (also shown in FIG. 23), respectively, are shown. In some implementations, inner catheter assemblies 766′ and 766″ are the same or similar to catheter assembly 766 shown in FIG. 56 and configured for implanting a posterior implant 610 (shown in FIG. 23), but are populated with fewer anchor driver tubes 818 and anchor leads 622, and fewer or no tether pullers 694. Each of these two inner catheters assemblies 766′ and 766″ may be provided with a driver tube 818 and anchor lead 622 running up the center of the control housing 812 as previously described for assembly 766, and a second set of driver tube 818 and anchor lead 622 extending from either the medial wing 816 or lateral wing 816, as appropriate for the particular implant. The control housing 812 in each case may be configured and function as previously described for inner catheter assembly 766. Each inner catheter assembly 766′ and 766″ may also be provided with an implant loader 686 or 762 (not shown) loaded with an anterior implant, similar to the arrangement for the posterior implant described above.

In some embodiments (not shown), the above-described instruments may be adapted to deliver implants having fewer or more anchors than the implants previously described. For example, a posterior implant may have only one medial anchor and one lateral anchor and therefore would not need all of the leads and driver tubes described above. Regardless of the number of medial and lateral anchors, a central anchor may be included or omitted. When the central anchor is omitted from a posterior implant, a central lead may still be used similar to as previously described to guide a torquer into place against the implant and help retain it there. An anterior implant may be provided with a single anchor, either within a torquer or separate therefrom. A lead may still be used through the torquer even when an anchor is not located there. In some embodiments, an anterior implant may be similar to a posterior implant and include a central anchor and two outboard anchors.

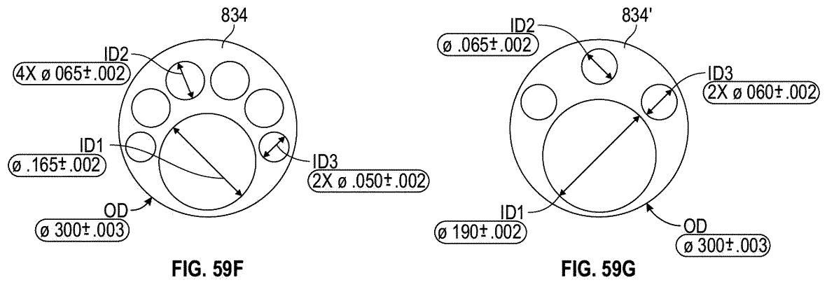

Referring to FIG. 59A, an exploded view is provided showing the interior components of inner steerable catheter assembly 766. FIG. 59B is a longitudinal cross-sectional view also showing the interior components of inner steerable catheter assembly 766. FIG. 59C is an enlarged cross-sectional view showing the distal/left portion of FIG. 59B. FIG. 59D is an enlarged cross-sectional view showing the middle portion of FIG. 59B. FIG. 59E is an enlarged cross-sectional view showing the proximal/right portion of FIG. 59B.

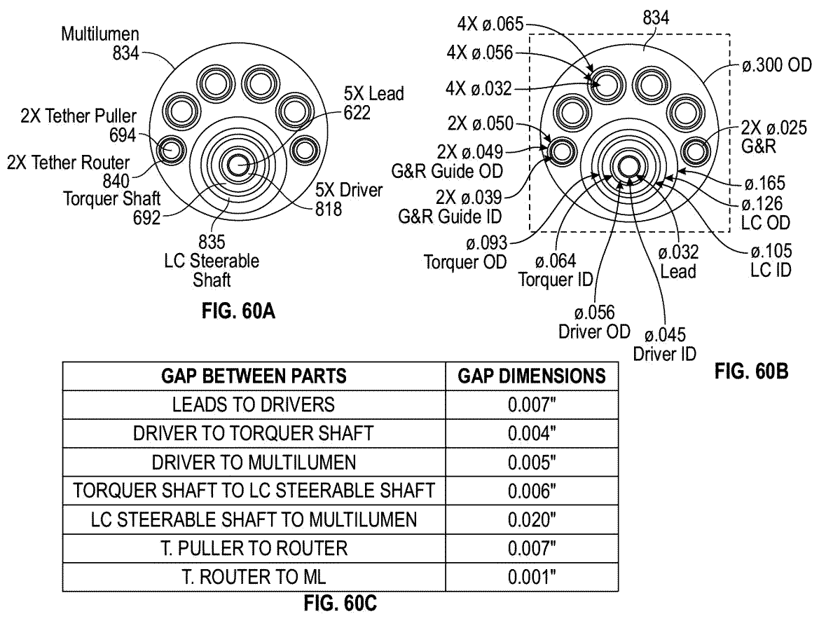

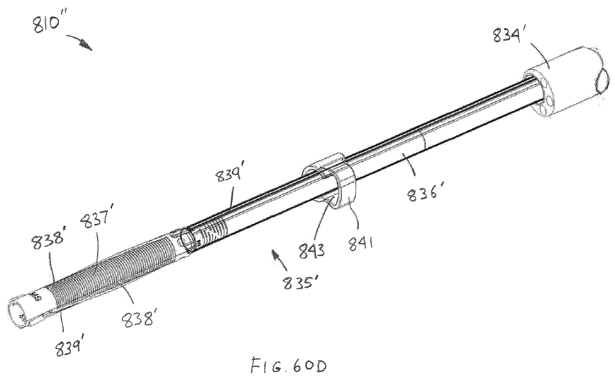

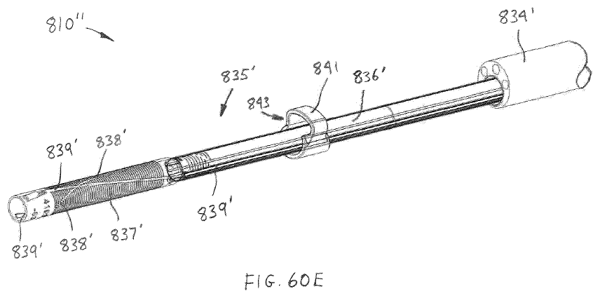

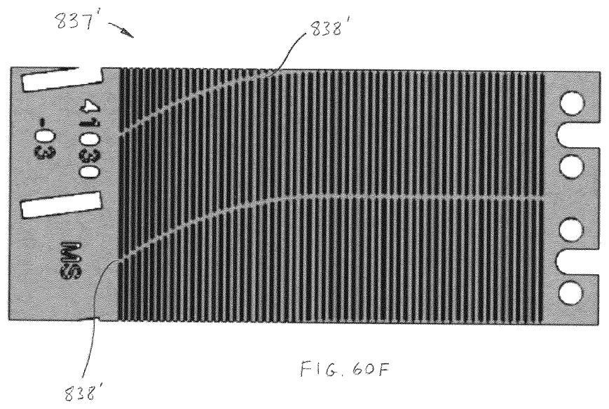

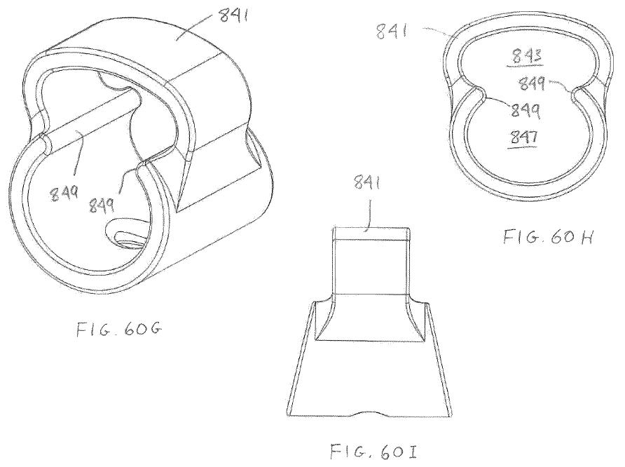

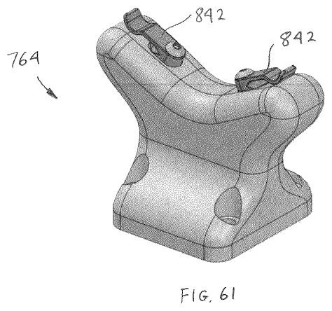

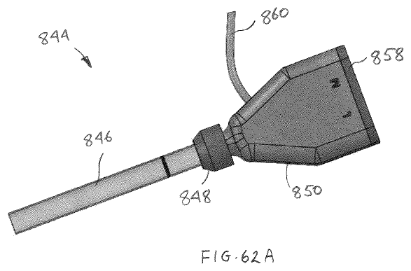

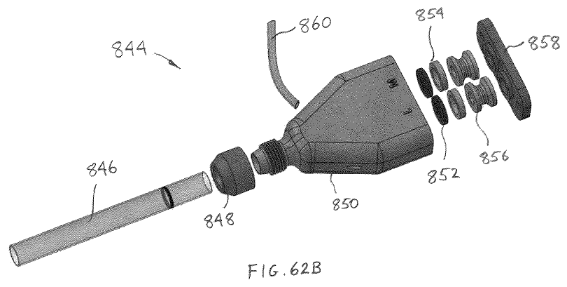

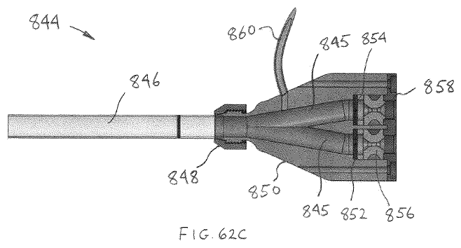

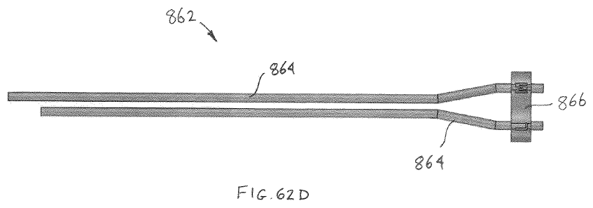

Referring to FIGS. 59F and 59G, cross-sections of inner catheters are shown. FIG. 59F shows inner catheter 810 of inner catheter assembly 766 and FIG. 59G shows inner catheters 810′/810″ of inner catheter assemblies 766′/766″. Referring first to FIG. 59F, inner catheter 810 may be provided with a multi-lumen extrusion (MLE) 834 having at least seven lumens therethrough. In this exemplary embodiment, a large hole is located at the bottom of MLE 834 with a crescent shaped series of smaller holes located along the top. Each of the holes is configured to receive at least one of a laser-cut steerable shaft 835, torque driver tube 692, anchor driver tube 818, anchor lead 622, tether 614, tether puller 694 and or tether router 840 therethrough, as shown in FIGS. 60A and 60B. Tether router 840 can provide a consistent lumen for a tether and or a tether puller to traverse the system, particularly from the back of MLE 834 to a manifold exit. MLE 834 keeps the above-listed items from becoming tangled or twisted with each other, can increase their responsiveness when they are being actuated. The MLE can greatly improve torque transmission of the inner system as a whole. In this exemplary embodiment, the proximal end of MLE 834 couples to the control housing. The proximal end of the laser cut steerable shaft 835 also couples to the control housing and its distal end, steered by pull wires from the control housing, extends beyond the distal end of MLE 834. FIG. 60B provides the dimensions the above items in this exemplary embodiment and FIG. 60C shows the gaps that extend between them. Referring again to FIG. 59G, a multi-lumen extrusion 834′ configured for use with inner catheter assemblies 766′/766″ may be similar to MLE 834 shown in FIGS. 59F, 60A and 60B and populated with the same items, but having only one lumen along its top for receiving a coaxial lead and driver. The lumens of MLE 834′ for the steerable shaft and for the tethers/tether pullers may also be larger than the corresponding lumens of MLE 834, as shown.

Referring to FIGS. 60D-60F, details of an exemplary laser cut inner steerable shaft 835 are shown. The steerable segment of inner steerable shaft 835 is constructed from two stainless steel hypo tubes and three laser cut patterns. The most proximal section 836 is a smaller diameter that runs roughly the length of MLE 834 and has a laser cut pattern to allow flexibility of the inner catheter in the vasculature. The smaller diameter section 836 running through MLE 834 allows the MLE a smaller diameter and therefore lower overall system profile. The exposed segment of the proximal hypo tube 836 (long smaller diameter) has a cut pattern that allows flexibility in all directions and the distal end of this section is intended to roughly align with the curved portion of the outer guide when the inner catheter is advanced into the atrium. The most distal section hypo tube 837 is larger in diameter and has a laser cut pattern that allows curvature in one plane (primary curvature). The larger most distal section 837 provides a larger diameter to allow a torquer to recess into the steerable assembly. This also provides support for the torquer. The larger diameter tube 837 also allows for higher torsional stiffness of the curved distal section (due to the larger moment of inertia) therefore providing enhanced control of the distal end. In some embodiments, the most distal section of the laser cut has a spine 838 that is spiraled. This provides some curvature roughly normal to the primary curvature. This pattern may be used on the trigone systems and allows the distal end to angle more anterior than the single primary curve. This spiral pattern (or opposite pattern) could also be used in the posterior system to increase or decrease the approach angle (in the anterior-posterior direction) of the implant to the annulus. In this exemplary embodiment, two spines 838 are provided along distal section 837, located 180 degrees apart. The maximum deflection of distal section 837 occurs between the two spines 838 and minimal to no deflection occurs along the spines. Two pull wires 839 are also provided along distal section 837 and also located 180 degrees apart from each other. As best seen in FIG. 60D, pull wires 839 (only one is visible) start out at the proximal end of distal section 837 being 90 degrees apart from spines 838. As spines 838 extend distally they spiral in one direction while pull wires 839 spiral in an opposite direction, crossing spines 838 before they reach the distal end of distal section 837. With this arrangement, distal section 837 is able to curve to a greater extent in a primary direction and to a lesser extent in a secondary direction generally perpendicular to the primary direction. In this exemplary embodiment, the inner catheter is steered with two guide wires (i.e., able to alternately curve in opposite directions) while the outer catheter is steered with a single guide wire (i.e., able to curve in a single direction.) In some embodiments, the laser-cut hypo tubes are covered with a braided jacketing and or polymer covering. In some embodiments, the pull wire(s) are sandwiched between the outer diameter of the hypo tube assembly and braided jacketing. FIG. 60F shows the laser cut pattern in distal tube 837 as if it were laid flat in a sheet.

FIGS. 60D and 60E also show an exemplary guide clip 841 that may be used on the lateral trigone system. Guide clip 841 is configured to clip onto inner steerable shaft 835 and is provided with an oval-shaped opening 843 configured to guide the coaxial anchor driver tube/anchor lead, tether, tether puller and or spring lumen (not shown) that extend distally from MLE 834, to keep them from twisting or tangling. In some embodiments, guide clip 841 is located about 60 mm proximal from the distal tip of steerable shaft 835, and about 35 mm distal from the distal end of MLE 834. In this exemplary embodiment, opening 843 is aligned with the small lumens on the top of MLE 834, as shown. In some embodiments (not shown), opening 843 is turned about 90 degrees counterclockwise from the small openings on the top of MLE 834 (as viewed from the proximal end of the instrument.)

Referring to FIGS. 60G-60I, enlarged views of guide clip 841 are shown. As best seen in FIG. 60H, guide clip 841 may be provided with a first oval-shaped opening 847 for receiving the inner steerable shaft 835 and an interconnected second oval-shaped opening 843 for guiding the above-mentioned elements. Clip 841 may be formed from a polymer or other resilient material such that inwardly protruding tabs 849 may be flexed outwardly when placing clip 841 over the inner steerable shaft. Tabs 849 can then provide an inward pressure to maintain the clip in place on the shaft. In this exemplary embodiment, clip 841 has an axial width that ranges from 3 to 5 mm. By keeping the width short, such as no more than 5 mm, clip 841 does not significantly affect the flexibility of the inner or outer catheters.

Referring to FIG. 61, and exemplary embodiment of a tether retaining yoke 764 is shown. As previously mentioned with regard to FIGS. 48A-48C, yoke 764 may be configured to mount on top of a linear carriage so that it may be moved along a rail between the proximal control handles of inner and outer catheters. In this exemplary embodiment, a spring clip 842 is attached to the top surface of each of the upward and laterally extending arms. Each spring clip 842 has a rounded distal tip that works in combination with the underlying arm's top surface to allow a tether to be easily slid into and out of the clip. In operation. spring clips 842 provide a location to temporarily store the proximal ends of tethers that extend out the proximal end of an outer catheter after one instrument has been removed from the outer catheter and before another instrument is inserted. This arrangement can keep a slight amount of tension on the tethers and keep them from crossing. In some embodiments, letters or other indicia (not shown) can be provided to label the medial and lateral sides of yoke 764 for holding the medial and lateral tethers. In alternative embodiments (not shown), additional clip(s) 842 are arranged on yoke 764 for use in procedures that involve more than two tethers.

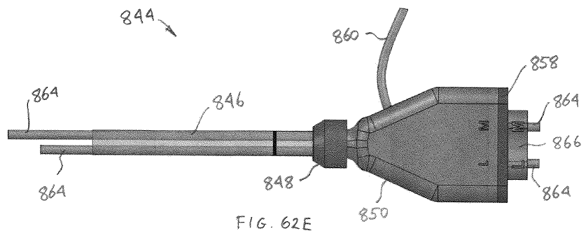

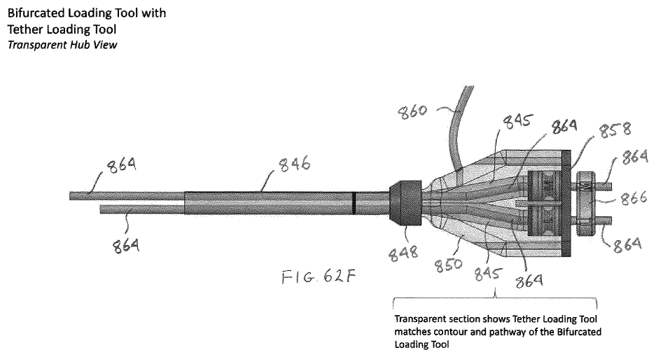

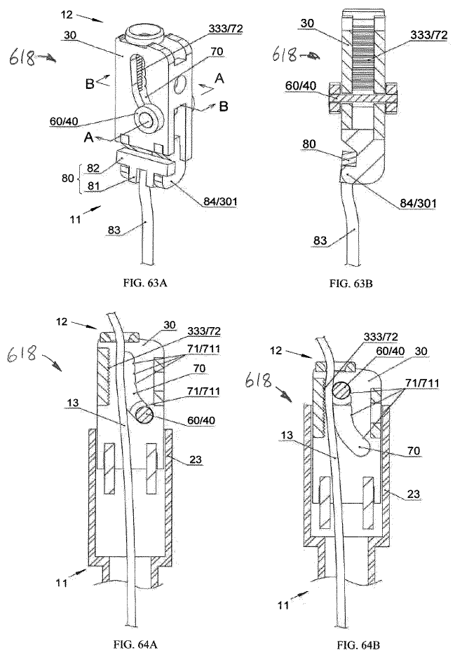

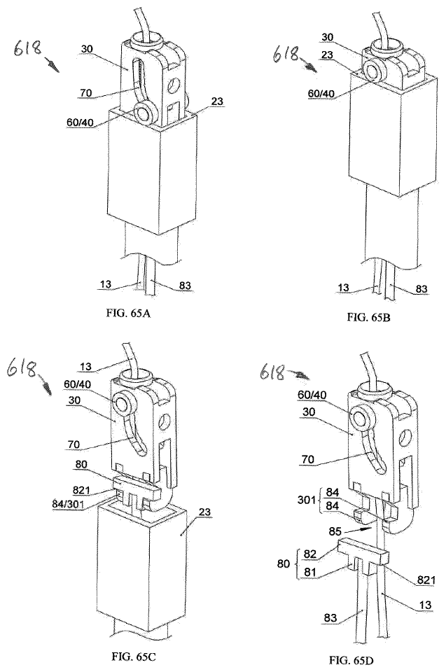

Referring to FIGS. 62A-62C, an exemplary embodiment of a bifurcated loading tool 844 is shown. In some implementations, after all devices have been implanted and the last inner catheter and loading tool has been removed from the outer catheter, the tethers from the implants may be threaded through loading tool 844 and it may be placed in the distal end of the outer catheter. Loading tool 844 may then be used to load tools over the tethers one at a time or simultaneously, such as for tensioning the tethers and cutting them off near the implants, as will be later described.

As best seen in FIG. 62C, exemplary loading tool 844 is provided with a pair of converging channels 845 that join together as they lead into the proximal end of canula section 846. The two sides may be labeled M for medial and L for lateral, as best seen in FIG. 62A. As best seen in FIG. 62B, loading tool 844 includes a cannula section 846, a collet nut 848, a housing 850, a first pair of seals 852, a second pair of seals 854, a pair of hemostasis valves 856, a retaining plate 858 and a flush port line 860. Collet nut 848 is configured to thread onto the distal end of housing 850 to secure canula section 846 thereto. One of the first seals 852, one of the second seals 854 and one of the hemostasis valves 856 are placed into each of the two channels 845 (FIG. 62C) and retained there by plate 858, which may be fastened to housing 850 by threaded fasteners (not shown.) Hemostasis valves 856 allow instrumentation to be inserted through channels 845 in tool 844 and through the outer catheter but prevent unrestricted blood flow when the instrumentation is removed. A flush port valve (not shown) may be provided on flush port line 860 to permit periodic flushing of tool 844.

Referring to FIGS. 62D-62F, an exemplary embodiment of a tether guide 862 is shown for use with bifurcated loading tool 844. Guide 862 is provided with two tubes 864 that are bent to match the inner contours of tool 844. One the distal ends of tubes 864 may be longer than the other for ease of inserting the tethers and to distinguish the two tubes from each other. The proximal ends of tubes 864 may be joined with a connector 866. Connector 866 and or tubes 864 may be slightly flexible and or configured to be rotatable with respect to one another so that they can follow the turns in channels 845 as they are being inserted through loading tool 844.

In operation, tether guide is placed into loading tool 844, as shown in FIGS. 62E and 62F. The proximal end of the medial tether extending from the distal end of the outer catheter is then inserted into the distal end and through the medial tube 864. Similarly, the proximal end of the lateral tether is inserted into the distal end and through the lateral tube 864. Guide 862 can then be removed through the proximal end of loading tool 844 and the tool can be inserted into the proximal end of the outer catheter. With this arrangement, the medial and lateral tethers can be easily inserted through their proper channels in loading tool 844, and without concern that they may be crossed.

In some embodiments (not shown), three or more converging channels 845 may be provided in loading tool 844 and three or more tubes 864 provided in tether guide 862 for use in procedures that involve three or more tethers.

Referring again to FIGS. 48A-48C, the overall operation of exemplary instrumentation system 750 will now be described. In some procedures, outer steerable catheter assembly 760 is moved from the upper proximal end 772 towards the lower distal end 774 of linear guide rail 768 as the distal end of outer steerable catheter assembly 760 is introduced into a patient's artery and advanced towards the patient's heart. As previously described, an inner dilator may be tracked over a guide and used to pass the distal end of outer steerable catheter assembly 760 through the septum of the heart and into the left atrium of the subject using a transseptal approach. Once outer catheter 760 is in place, tether retainer 764 may be installed onto guide rail 768 and or slid into position several inches proximal of the proximal end of outer catheter 760.

A separate, steerable inner catheter assembly and implant loading tool 762 combination for each device to be implanted may now be introduced in sequence through the outer steerable catheter assembly 760. In some implementations, inner catheter assembly 766′ (FIG. 57) is used first to implant a medial anterior implant, followed by inner catheter assembly 766″ (FIG. 58) to implant a lateral anterior implant, followed by inner catheter assembly 766 (FIG. 56) to implant a posterior implant. Each inner catheter assembly 766′, 766″ and 766 may in turn be snapped onto and later removed from the same carriage assembly 770.

Inner catheter 766′ may include tether 614 with its distal end pre-attached to a pre-loaded medial implant 612 (both shown in FIG. 23) and its proximal end extending from medial wing 816, as shown in FIG. 57.

The distal end of inner steerable catheter assembly 766′ may now be passed through outer catheter 760. In this exemplary implementation, implant loading tool assembly 762 is packaged on the distal end of inner steerable catheter assembly 766′ with a medial implant preloaded into it. Carriage assembly 770 for inner steerable catheter assembly 766′ may be attached or previously located on the upper proximal end 772 of guide rail 768 and moved distally until the distal end of implant loading tool assembly 762 reaches the proximal end of outer catheter 760. The distal end of tool 762 may then be inserted into outer catheter 760 and then locked in place. The proximal end of inner steerable catheter assembly 766′ may then be moved from the upper proximal end 772 towards the lower distal end 774 of linear guide rail 768 as the distal end of inner steerable catheter assembly 766′ and its associated implant is introduced through loading tool assembly 762 into the proximal end of outer catheter 760. Once the implant and distal end of inner catheter 766′ are advanced through outer catheter 760 and emerge from its distal end into the subject's left atrium as previously described, the proximal ends of inner catheter 766′, loading tool 762 and outer catheter 760 are positioned relative to one another approximately as shown in FIGS. 48A-48C.

After the medial anterior implant is implanted as previously described, inner catheter assembly 766′ may be withdrawn from outer catheter assembly 760 and removed from carriage assembly 770. At this point, the proximal end of the tether extending from the implanted medial implant will be protruding from the proximal end of the outer catheter and may be attached to the medial clip of tether retaining yoke 764. The inner catheter assembly 766″ (FIG. 58) for the lateral implant may now be attached to the carriage assembly 770 used for the inner catheters. As shown in FIG. 58, inner catheter assembly 766″ may be provided with a tether 614 attached to its lateral implant and extending from lateral wing 816. Inner catheter assembly 766″ may also be provided with a tether puller 694 extending from medial wing 816. The distal end of tether puller 694 may now be attached, as previously described, to the proximal end of the previously mentioned tether from the implanted medial anterior implant. Tether puller 694 may be proximally withdrawn from inner catheter assembly 766″ bringing the tether with it, and may then be removed from the tether such as by cutting off the proximal-most end of the tether. Inner catheter assembly 766″ and the implant loading tool 762 attached to it may now be introduced into outer catheter 760 after the tether for the medial implant is unclipped from yoke 764. Slight tension should be kept on the tether to ensure that inner catheter 766″ tracks over it rather than it bunching up in the outer catheter.

After the lateral anterior implant is implanted as previously described, inner catheter assembly 766″ may be withdrawn from outer catheter assembly 760 and removed from carriage assembly 770. At this point, the proximal ends of the tethers extending from the implanted medial and lateral implants will be protruding from the proximal end of the outer catheter and may be attached to the medial and lateral clips, respectively, of tether retaining yoke 764. The inner catheter assembly 766 (FIG. 56) for the posterior implant may now be attached to the carriage assembly 770 used for the inner catheters. As shown in FIG. 56, inner catheter assembly 766′ may be provided with a first tether puller 694 extending from medial wing 816 and a second tether puller 694 extending from lateral wing 816. The distal ends of tether pullers 694 may now be attached, as previously described, to the proximal ends of the previously mentioned tethers from the implanted medial and lateral anterior implants. Tether pullers 694 may be proximally withdrawn from inner catheter assembly 766 bringing the tethers with them, and may then be removed from the tethers such as by cutting off the proximal-most ends of the tethers. Inner catheter assembly 766 and the implant loading tool 762 attached to it may now be introduced into outer catheter 760 after the tethers for the medial and lateral implants are unclipped from yoke 764. Slight tension should be kept on the tethers to ensure that inner catheter 766 and the associated posterior implant track over them rather than the tethers bunching up in the outer catheter.

After the posterior implant is implanted as previously described, inner catheter assembly 766 and its loading tool 762 may be withdrawn from outer catheter assembly 760 and removed from carriage assembly 770. At this point, the proximal ends of the tethers extending from the implanted medial and lateral anterior implants and passing through the implanted posterior implant will be protruding from the proximal end of the outer catheter and may again be attached to the medial and lateral clips, respectively, of tether retaining yoke 764.

Bifurcated loading tool 844 (shown in FIGS. 62A-62F) may now be used. The proximal ends of the tethers from the implanted medial and lateral anterior implants may be threaded through loading tool 844, as previously described. Loading tool 844 may now be introduced into the proximal end of outer catheter 760 after the tethers have been unclipped from yoke 764. Slight tension should be kept on the tethers to ensure that tool 844 tracks over them rather than the tethers bunching up.

Additional implants and or instrumentation may now be passed through bifurcated loading tool 844 and through outer catheter 760. In particular, tether locks 618 (shown in FIGS. 23, 24 and 63A-65D) can be tracked over the tethers one at a time. A separate instrument (disclosed in more detail below) may be passed through loading tool 844 to introduce a tether lock 618 over each tether and to apply a desired amount of tension to each tether. Such an instrument may be alternated between the tethers to tension and or re-tension the tethers until the desired amount of tension (which may be patient specific, and which may be different between the tethers) is obtained. Alternatively, two tensioning/locking instruments may be used simultaneously, one over each tether. In some implementations, imaging and or real-time measurements may be taken to evaluate the tether tensioning as it is being performed. Once the desired tensions are achieved, the tensioning/locking instruments may be removed and a tether cutting instrument (disclosed in more detail below) may be passed through loading tool 844 and over each tether one at a time to cut off the excess length of the tethers, such as just proximal to a tether lock. After confirmation that the implant system has been properly implanted, outer catheter assembly 760 is slid proximally along guide rail 768 to withdraw the outer catheter from the patient.

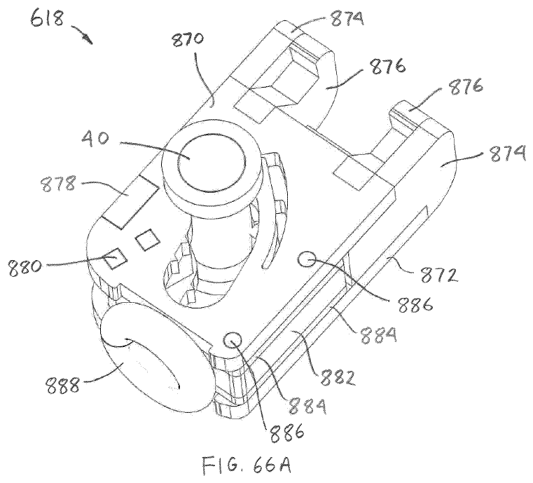

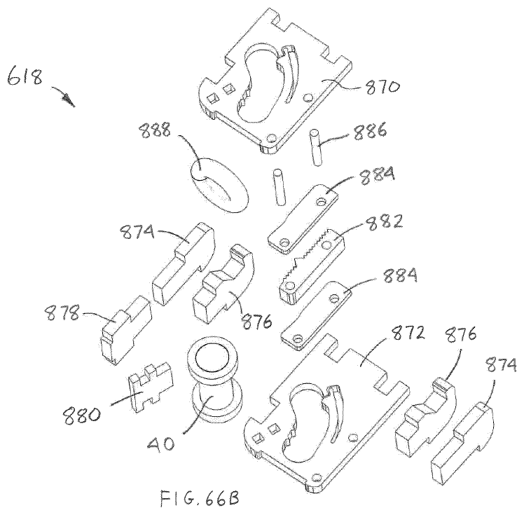

Referring to FIGS. 63A-65D, further details of an exemplary tensile member/tether lock 618 are shown. The locking device includes: a lock body 30 and a movable part 40. The lock body 30 is configured for the tensile member 13 to pass through, and the lock body 30 is provided with a sliding groove 70. The movable part 40 can be movably installed in the sliding groove 70. The lock body 30 is provided with an abutting part 333, and the tensile member 13 is located between the abutting part 333 and the movable part 40. The movable part 40 is configured to move along the sliding groove 70 to the abutting portion 333 under the action of an external force to limit the tensile member 13 to the location of the abutting portion 333.

The sliding groove 70 provides a guiding function for the movable part 40, and the movable part 40 moves along the sliding groove 70 under the action of external force, and gradually approaches the abutting portion 333, thereby limiting the tensile member 13 to the abutting portion 333. As shown in FIGS. 64A and 64B, the tensile member 13 passes through the lock body 30 along the direction from the proximal end 11 to the distal end 12. The sliding groove 70 acts as a chute, and the sliding groove 70 extends along the direction from the proximal end 11 to the distal end 12. The direction is inclined toward the abutting portion 333. Applying a force in the direction from the proximal end 11 to the distal end 12 on the movable part 40 can drive the movable part 40 to move along the sliding groove 70 toward the abutting part 333, and clamp the tensile member 13 between the abutting part 333 and the movable part 40 by putting pressure on it. Preferably, the movable member 40 is a pin 60 penetrating through the sliding groove 70 and having an enlarged head portion at both ends to retain pin 60 in groove 70.

Further, the abutting portion 333 may be provided with a series of teeth on an abutting surface 72 to increase the friction between the tensile member 13 and the abutting portion 333 and to improve the stability of the locking tensile member 13.

In one embodiment, the inner wall of the sliding groove 70 is provided with a limiting portion 71. As the movable member 40 moves along the sliding groove 70 from the proximal end 11 to the distal end 12, the movable member 40 can pass the limiting portion 71 under the action of external force and continue to move distally. After member 40 moves into place and the external force is removed, the limiting portion 71 limits the movable member 40, and the limiting portion 71 prevents the movable member 40 from returning to the proximal end 11, thereby locking the relative position between the lock body 30 and the movable member 40. The stability of the locking tensile member 13 is therefore improved. The limiting portion 71 may be located on the inner side wall of the sliding groove 70 away from the abutting surface 72. Specifically, the limiting portion 71 may be a limiting convex point or a limiting rib, etc. Referring to FIGS. 64A and 64B, in this exemplary embodiment the position limiting portion 71 is a limiting rib 711 extending longitudinally along the movable member 40. Further, a plurality of limiting ribs 711 are provided at the distal end of the inclined chute 70, and the movable part 40 can be limited to different limiting ribs 711 according to the thickness of the tensile member 13, so that the locking device is suitable for different thicknesses. The provision of multiple limit ribs 711 can also enable an operator to push the movable part 40 to the distal end without continuous force when the operator is exerting a force in the proximal direction against pulling member 13, thereby reducing the difficulty of operation. Further, the proximal end of the inner wall of the sliding groove 70 can also be provided with a limiting portion 71 for preventing the movable part 40 from moving to the distal end, so that the movable part 40 is initially retained at the proximal end of the sliding groove 70, which is convenient for sliding the lock body 30 along the tensile member 13 without resistance.

As shown in FIGS. 65A and 65B, the external force can be provided by a sliding cylinder, sleeve or collar 23. The sliding collar 23 is sleeved over the lock body 30, and the sliding collar 23 abuts against the movable part 40. The sliding collar 23 serves as the locking driving device 20. Sleeve 23 moves from the proximal end 11 to the distal end 12 relative to the lock body 30, thereby pushing the movable part 40 to move toward the abutting portion 333. In this exemplary embodiment, the sliding collar 23 is detachably connected to the locking device. After the locking of the tensile member 13 has been completed, the sliding collar 23 can be withdrawn from the locking device.

In this exemplary embodiment, as shown in FIGS. 63A and 63B, the lock body 30 is provided with a lock body connecting portion 301. The lock body connecting portion 301 is located at the proximal end of the lock body 30 and includes at least one connecting protrusion 84. The locking device also includes a chuck 80 provided with a connecting beam 82. The connecting protrusion 84 and the connecting beam 82 are configured to be detachably connected. The connecting beam 82 and the connecting protrusion 84 are also configured to connect the protrusion 84 to a proximally applied force to prevent the lock body 30 from moving when the movable member 40 receives an external distally applied force. As shown in FIGS. 63A, 63B, 65C and 65D, the lock body 30 is fixed, the sliding collar 23 pushes the movable member 40 to move toward the abutting portion 333 in the direction from the proximal end 11 to the distal end 12. During the process, the chuck 80 can apply a force to the lock body 30 in the proximal direction through the coupling of the connecting beam 82 and the connecting protrusion 84 to keep the position of the lock body 30 stable. The chuck 80 can be connected to pull wire 83 to apply a pulling force to the lock body 30 through pull wire 83 and chuck 80. In some embodiments, the lock body 30 remains stationary while sliding collar 23 is moved distally by the catheter to actuate the lock. In some embodiments, sliding collar 23 remains stationary while lock body 30 is moved proximally by pull wire 83 to actuate the lock.

As shown in FIG. 65C and FIG. 65D, the chuck 80 and the lock body connecting portion 301 have a detachable structure, which facilitates assembly and cooperation, and the chuck 80 is evacuated after the tensile member 13 is locked. Specifically, the pull wire 83 may be a filamentous material, a belt, a string, a cable, a rod or a suture. In some embodiments, pull wire 83 includes a flexible and/or superelastic material, for example, Nitinol, polyester, stainless steel, or cobalt-chromium alloy. In some embodiments, the pull wire 83 may also be a rigid or semi-rigid rod-shaped construction, such as stainless steel.

Further, the lock body connecting portion 301 includes a plurality of connecting protrusions 84 distributed at intervals, and a connecting groove 85 is provided between the plurality of connecting protrusions 84. In this exemplary embodiment, the chuck 80 includes a connecting post 81 fixed to the connecting beam 82, and the connecting post 81 is inserted in the connecting groove 85, which facilitates the connection between the chuck 80 and the lock body connecting portion 301 more reliably, and facilitates assembly and disassembly.

Further, as shown in FIGS. 63B, 65C, and 65D, the connecting protrusion 84 is configured in an L-shape. The connecting beam 82 is provided with an abutting surface 821 that abuts the connecting protrusion 84. The connecting beam 82 and the connecting column 81 form the T-shaped chuck 80. This ensures that the lock body connecting portion 301 and the chuck 80 have a large contact area, which is beneficial to transferring the pulling force.

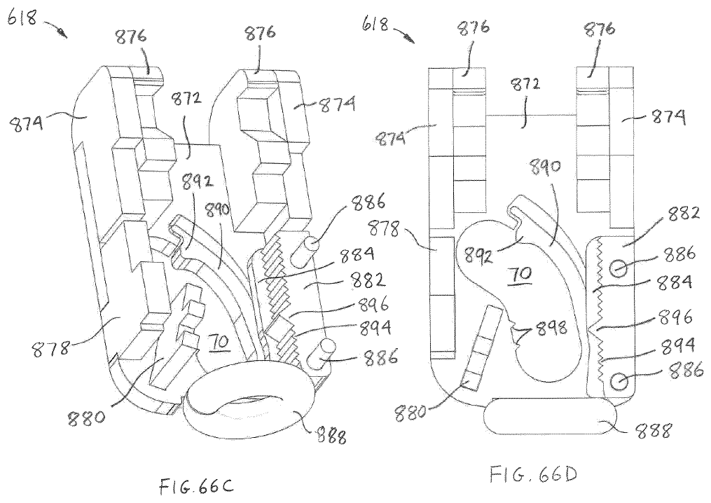

Referring to FIGS. 66A-66D, additional features found in an alternative embodiment of tether lock 618 are shown. In this exemplary embodiment, lock 618 includes front plate 870, back plate 872, a pair of laterally spaced earflaps 874, a pair of laterally spaced coupling plates 876, a side plate 878, a H-shaped brace 880, a toothed abutment block 882, a pair of spaced apart backstop plates 884, a pair of backstop pins 886, a tether bushing 888, and a movable member/lock pin 40.

As best seen in FIGS. 66C and 66D, lock 618 may be provided with a flexure arm 890 forming and or adjacent to one side of sliding groove 70. An inwardly protruding tooth 892 may be provided near the free end of flexure arm 890. In an unflexed state as shown in FIGS. 66C and 66D, tooth 892 protrudes into sliding groove 70 and inhibits movable member/lock pin 40 (shown in FIGS. 66A and 66B) from moving from the proximal end of groove 70 to the distal end of groove 70. When an external force is applied to pin 40 as previously described, the pin abuts against tooth 892 and causes arm 890 to flex away from groove 70, allowing pin 40 to pass towards the distal end of groove 70. This arrangement allows the tether to freely slide through lock 618 until the operator is ready to set the lock.

In this exemplary embodiment, abutment block 882 is provided with a series of teeth 894 to better engage with the tether when in the locked state. As previously described, the tether may have a fiber core covered by an outer jacket. One large tooth 896 may be located in the series as shown to help grip into the fiber core with less potential to damage the outer jacket of the tether. In this embodiment, the large tooth 896 is located proximally from the center of pin 40 when it is slid to the end of groove 70 to help retain the pin in this locked position. In this embodiment, large tooth 896 has a height that is more than twice the height of the other teeth in the series 894.

As previously described, channel 70 may be provided with several teeth 898 located on the opposite side of groove 70 from abutment block 882, as shown in FIGS. 66C and 66D. Teeth 898 can let the operator move the lock pin toward the locked position without the need to maintain a constant force on the lock pin, and can serve to keep the lock pin from moving out of the locked position.

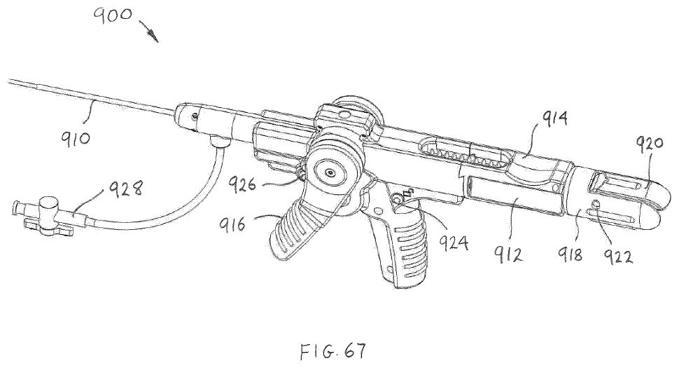

Referring to FIGS. 67-72, an exemplary instrument 900 configured to install and tension the above-described tether locks is shown. Referring first to FIG. 67, the proximal handle section of instrument 900 is shown. Instrument 900 includes a catheter section 910 and a main handle housing made up of a left half 912 and a right half 914, which may be secured together with fasteners. A handle trigger 916 may be provided with an upper forked section that is pivotably mounted to the left and right halves of the main housing as shown. A tension clamp base 918 may be provided at the proximal end of the housing with a tension clamp lever 920 pivotably mounted to it with a pivot pin 922 for clamping onto a tether. A trigger pull lock 924 may be slidably mounted in the main housing proximal to trigger 916 such that the lock slides transversely with respect to a longitudinal axis of the housing. A trigger push lock 926 may be slidably mounted in the main housing distal to trigger 916 such that the lock slides transversely with respect to the longitudinal axis of the housing. A flush port 928 may also be provided at the distal end of the main housing as shown.

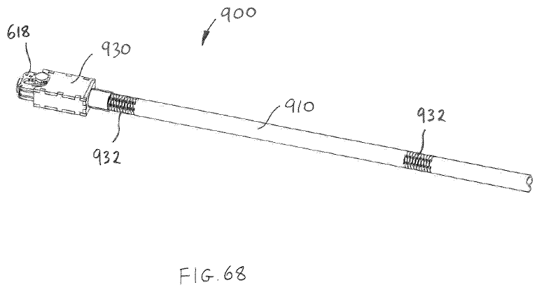

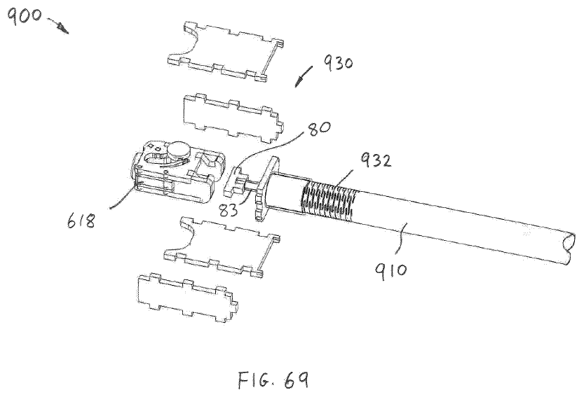

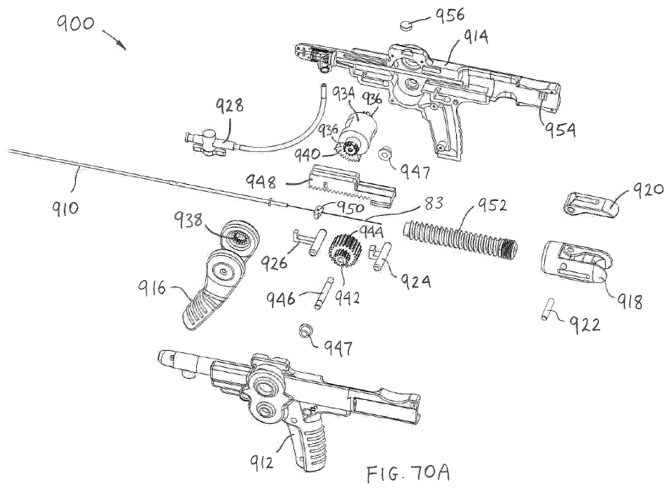

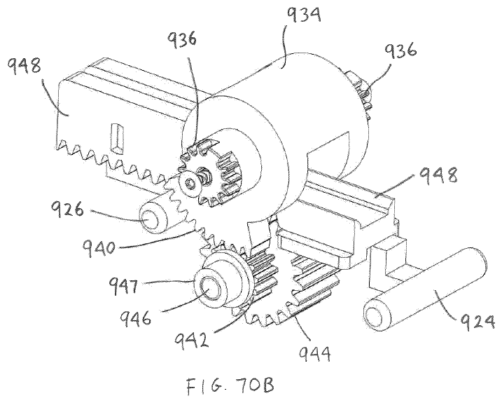

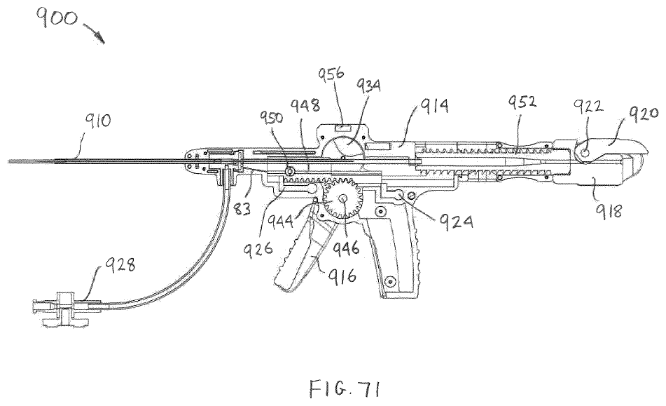

Referring to FIG. 68, the distal end of instrument 900 is shown. A collar 930 may be rigidly mounted to the distal end of catheter section 910 for slidably receiving the proximal end of tether lock 618 as shown. In some embodiments (not shown), a curved shroud or baseplate may be provided on the proximal end of collar 930 to ensure it does not snag when being withdrawn from the patient. One or more laser-cut sections 932 may be provided near the distal end of the catheter as shown to allow it to be more flexible when positioning lock 618 against an implant.

Referring to FIG. 69, the distal end of instrument 900 is shown with the components of collar 930 displayed in an exploded manner so that lock 618 and the coupling component 80 at the end of the pull wire 83 can be fully seen.

Referring to FIG. 70A, an exploded view of the proximal handle section of instrument 900 shows components thereof. A straddle gear 934 may be pivotably mounted between the left handle housing 912 and the right handle housing 914. In this exemplary embodiment, straddle gear 934 (also shown in FIG. 70B) is provided with a pinion gear 936 on either side and configured to engage with a mating recess 938 inside each upper end of trigger 916. Straddle gear 934 is also provided with a pair of laterally spaced apart gear segments 940 arranged to drive pinion gears 942. Pinion gears 942 are rigidly attached to drive gear 944 and all are rotatably mounted on gear shaft 946 and bushings 947 between the left handle housing 912 and the right handle housing 914 beneath gear rack 948. With this arrangement, when trigger 916 is squeezed (i.e., pivoted in a proximal direction), gear rack 948 slides proximally between the left handle housing 912 and the right handle housing 914. Pull wire 83 is coupled to gear rack 948 with cable lock 950 so that it is pulled proximally in catheter 910 when gear rack 948 slides proximally. In some embodiments, a return spring (not shown) is provided to bias trigger 916 and gear rack 948 towards a forward neutral position.

In this exemplary embodiment, tension clamp base 918 is threadably attached to a tension lead screw 952, which in turn engages with internal threads 954 located inside the left handle housing 912 and the right handle housing 914. A neodymium magnet 956 may be provided in a recess in the top of the handle housing as shown for removably coupling the housing to a linear slide carriage, as further described below.

Referring to FIG. 71, a longitudinal cross-section of instrument 900 is shown.

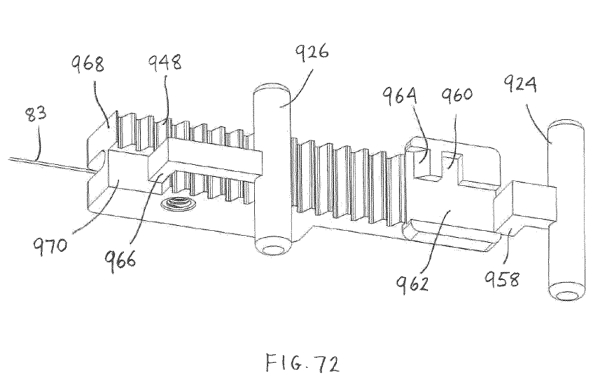

Referring to FIG. 72, an enlarged perspective view shows gear rack 948 from below. Also shown are trigger pull lock 924 and trigger push lock 926 in their unlocked positions. Gear rack 948 is shown in the distal-most position of its travel. During operation, gear rack 948 starts in a more proximal position (to the right in FIG. 72) and trigger pull lock 924 and trigger push lock 926 are in their locked positions (moved upward in FIG. 72.)

With gear rack 948 in the starting position, trigger pull lock pawl 958 engages with recess 960 in the bottom of gear rack 948, preventing the rack from moving proximally or distally until pawl 958 is slid (downwardly in FIG. 72) into alignment with groove 962. If trigger pull lock 924 is re-engaged after gear rack 948 is moved proximally, ramp 964 allows pawl to return to recess 960 when gear rack 948 returns distally, again locking gear rack 948 in its starting position.

With gear rack 948 in the starting position, trigger push lock pawl 966 engages with a distal surface 968 of gear rack 948, preventing the rack from moving distally until pawl 966 is slid (downwardly in FIG. 72) into alignment with groove 970. In this unlocked position, the trigger may be moved distally to drive gear rack 948 distally as shown, thereby pushing the pull wire 83 distally to disengage it from the implanted lock.

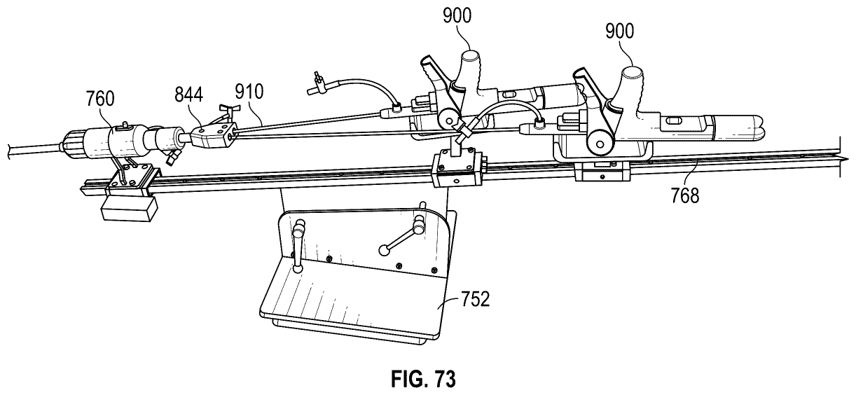

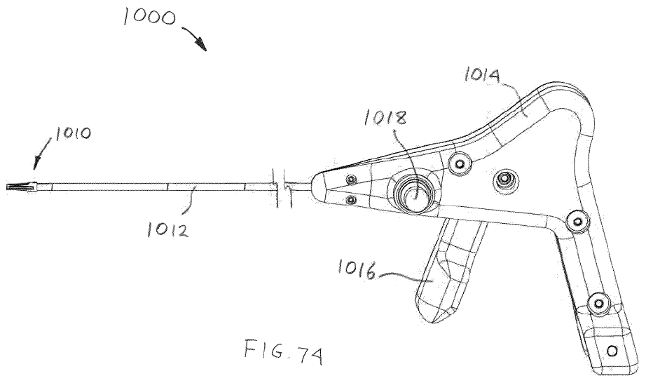

Referring to FIG. 73, operation of tether tensioning and locking instrument 900 will be described. In some implementations, instrument 900 is configured to be disposable after it is used in one angioplasty procedure. It may be provided in sterile packaging with a tether lock 618 preloaded on its distal end and retained there by coupling member 80 on the distal end of pull wire 83 (shown in FIG. 69.) In operation, instrument 900 is first removed from its packaging. The proximal end of a tether emanating from an implanted anterior implant, through an implanted posterior implant, through an outer catheter and through one side of a bifurcated loading tool, as previously described, may now be threaded through the preloaded tether lock, through instrument 900 and out its proximal end. The distal end of instrument 900 may be inserted through the bifurcated loading tool and through the outer catheter until the preloaded lock contacts one of the eyelet assemblies 620 of the posterior implant 610, as shown in FIG. 23. Instrument 900 can be inverted and releasably mounted to a carriage on linear guide rail 768 as shown, such as by using a magnet located in the top of the instrument housing, as previously shown and described. In some implementations, a second instrument 900 with its own preloaded tether lock 618 may now be advanced over the second tether in the same manner so that both instruments 900 can be used to tension the tethers at the same time. This allows for real-time feedback (such as from an echocardiogram) on the effect the tether tensioning is having on improving mitral valve coaptation. The second instrument 900 can also be inverted and releasably mounted to a second carriage on linear guide rail 768 with a magnet, as shown. In some embodiments, the catheter sections 910 of the two (or more) instruments 900 can be different lengths to allow the positions of the instruments to be staggered along the same guide rail 768, as shown.

After instrument(s) 900 is/are in place, tension clamp lever 920, previously placed in a vertical position, is lowered into a horizontal position (as shown in FIG. 71) to lock the tether against the tension clamp base 918. The tension clamp base 918 may then be rotated with respect to the rest of instrument 900 to advance the clamp base 918 in a proximal direction, thereby increasing the tension on the tether. Once the desired tension is obtained in both/all tethers (as confirmed by an echocardiogram in some implementations), trigger pull lock 924 may be disengaged by pushing on its left end protruding out of left handle housing 912. Trigger pull lock 924 may be reengaged by pushing on its right end which will now be protruding out of right handle housing 914. With trigger pull lock 924 disengaged, trigger 916 can now be squeezed causing gear rack 948 to move pull wire 83 proximally, as previously described with reference to FIGS. 67-72. This in turn will cause lock 618 to move from the unlocked state to the locked state, as previously described with reference to FIGS. 63A to 66D. Once both/all locks 618 have been locked, the desired coaptation of the mitral valve leaflets can again be confirmed. If for some reason it is desired to change the tension on a tether at this point, its lock 618 may be moved from the locked state back to the unlocked state. This may be accomplished by returning trigger 916 to its original position and pulling proximally on the tether, such as by rotating tension clamp base 918, thereby moving movable member 40 proximally away from abutting surface/block 72/882 (shown in FIGS. 63A to 66D.)

Once locks 618 have been set, instrument(s) 900 may be disengaged and removed. This may be accomplished by first moving trigger push lock 926 to the unlocked state, by pushing on its left end protruding out of left handle housing 912. Trigger pull lock 926 may be reengaged by pushing on its right end which will now be protruding out of right handle housing 914. With trigger pull lock 926 disengaged, trigger 916 can now be extended distally past its starting position causing gear rack 948 to move pull wire 83 distally, as previously described with reference to FIGS. 67-72. This in turn will cause T-shaped chuck/coupling member 80 to push lock 618 out of collar 23 and disengage from connecting portion/coupling element 301, as previously described with reference to FIGS. 65C and 65D. Instrument 900 may now be removed from the outer catheter and bifurcated loading tool, leaving its tensioned and locked tether remaining.

After locks 916 have been installed and instrument(s) 900 removed, a tether cutting instrument (described in more detail below) may be passed through the bifurcated loading tool and over each tether one at a time to cut off the excess length of the tethers, such as just proximal to a tether lock. After confirmation that the implant system has been properly implanted, the outer catheter assembly may be slid proximally along its guide rail to withdraw the outer catheter from the patient.

In initial embodiments of tether cutting instruments (not shown) developed by the present Applicant, the Applicant encountered and overcame a number of challenges. Guillotine style cutters have been described previously in the prior art. A basic design having a typical razor blade between adjacent openings that slides past the openings was found suitable to cut typical suture or monofilament tethers but would not cut the high strength fibers used in the tether described above of the present system. Design modifications were made that would allow the cutting system to cut through the majority of the tether, but high-strength fibers such as Dyneema are tenacious and would always leave some filaments that were not cut. It has been assumed that the same problem would occur with other high strength fibers such as Kevlar and Vectran.

To address this challenge, a backstop was added to provide a hard surface for the blade to contact and provide a surface for the blade to cut through the final high strength fibers. This interim design would sometimes cut the tether but very unreliably.

The Applicant then tried using sharper razor blades (microtome blades) which would cut somewhat better but still not reliably, and could dull during handling in manufacturing which would prevent cutting of the tether.

The Applicant then searched for harder cutting blades, which resulted in using tungsten carbide. Tungsten carbide blades would reliably cut the tether but could easily shatter, especially when welded to a blade holder.

To address this, the cutter system design was further modified to entrap the blade within the blade holder without welding. This reduced shattering, but it was still an issue.

The design was further changed to disperse the load on the blade by using a pin to evenly pull on the back of the blade. This further reduced occurrences of blade shattering. However, if the handle was squeezed too hard, the resulting force on the blade and backstop would cause the blade to shatter.

Accordingly, the Applicant incorporated a stop feature in in the blade holder that limits the amount of travel of the blade relative to the backstop. It was discovered that an interference of 0.0015″ between the blade and the backstop allows enough compression to cut the tether but does not generate enough force to shatter the blade. The exemplary embodiment of a tether cutting instrument disclosed below addresses all of the above issues, in addition to others, and represents many design iterations by the Applicant.

Referring to FIGS. 74-86, an exemplary instrument 1000 configured to thread over and cut a tether in vivo, such as adjacent to an above-described tether lock after tensioning, is shown. Referring first to FIG. 74, tether cutting instrument 1000 comprises a tether cutter assembly 1010 located at its distal end. Cutter assembly 1010 is coupled to the distal end of a flexible catheter 1012. In some embodiments, catheter 1012 is about 60 inches long. A handle section 1014 is coupled to the proximal end of catheter 1012 and is provided with a trigger or movable actuator 1016 for actuating cutter assembly 1010. Handle section 1014 may also be provided with a flush port 1018.

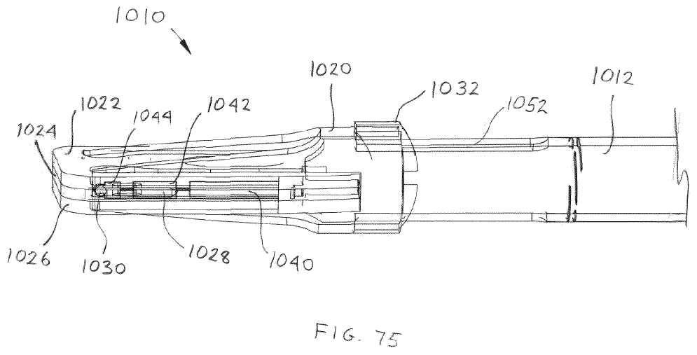

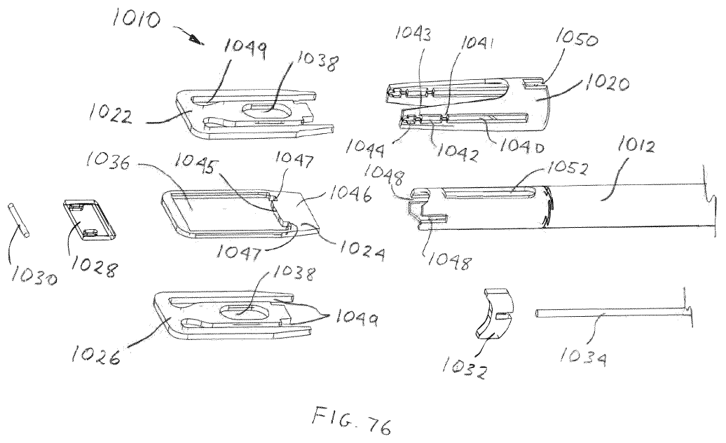

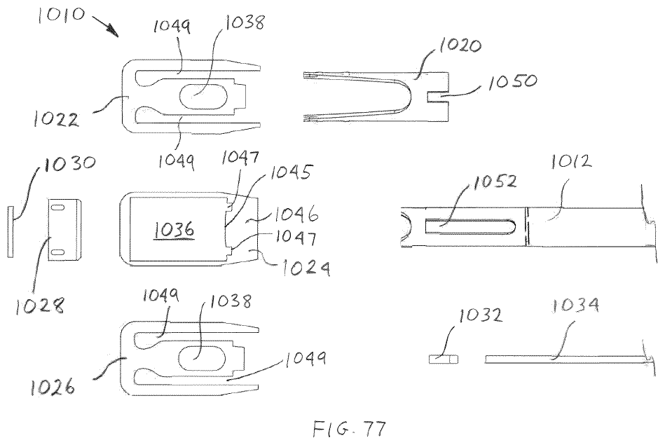

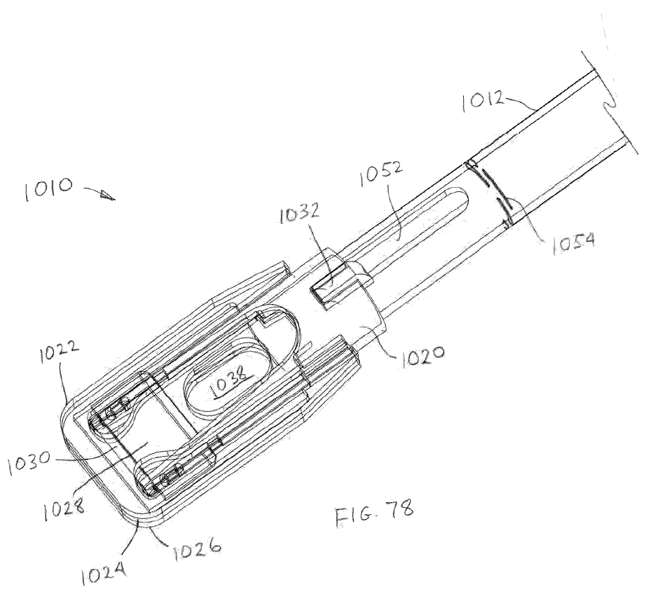

Referring to FIGS. 75-77, components of an exemplary cutter assembly 1010 are shown. FIG. 75 is a semi-transparent perspective view showing the distal end of instrument 1000, FIG. 76 is an exploded perspective view, and FIG. 77 is an exploded top plan view. Cutter assembly 1010 includes blade holder 1020, upper stationary plate 1022, filler plate 1024, lower stationary plate 1026, cutting blade 1028, pull pin 1030 and pull wing 1032. The distal end of a pull wire 1034 is also shown in FIGS. 76 and 77. Pull wire 1034 is configured to extend through a central lumen of catheter 1012 for connecting cutter assembly 1010 at the distal end of catheter 1012 to the handle section at the proximal end.

When the above components of cutter assembly 1010 are assembled, cutter blade 1028 is slidably received in window 1036 (shown in FIGS. 76 and 77) of filler plate 1024. Filler plate 1024 is sandwiched between upper stationary plate 1022 and lower stationary plate 1026, and also remains stationary. This arrangement slidably captivates blade 1028 in window 1036 and allows movable blade holder 1020 to drive blade 1028 in a proximal direction to cut a tether (not shown) when the tether extends through apertures 1038 in stationary plates 1022 and 1026.

As best seen in FIG. 76, in this exemplary embodiment, blade holder 1020 has a generally tubular shape and is configured to be slidably received over the distal end of catheter 1012. As previously mentioned, blade holder 1020 moves longitudinally relative to catheter 1012 to drive blade 1028 from a distal position towards a proximal position. As shown in FIG. 76, blade holder 1020 has a pair of longitudinally extending arms and a majority blade holder 1020 tapers towards a distal end. These longitudinally extending arms may be provided with a pair of opposing long slots 1040, a pair of opposing medium slots 1042 and a pair of opposing short slots 1044. Posts 1041 are formed between slots 1040 and 1042, and posts 1043 are formed between slots 1042 and 1044. These posts formed between the slots may be provided with longitudinal slits or gaps such that the top and bottom portions of the longitudinal arms may be pried apart when assembling cutter assembly 1010. During assembly, the proximal, laterally extending portion 1046 of filler plate 1024 is placed across the long slots 1040 such that slots 1040 are able to slide longitudinally with respect thereto. Once assembled, the lateral sides of the proximal portion 1046 extend outside of blade holder 1020, as does the remainder of filler plate 1024 which extends towards and around the distal end of blade holder 1020.

Cutter blade 1028 may be provided with a pair of opposing slots therethrough, as shown in FIGS. 76 and 77. The posts 1043 of blade holder 1020 (located between medium slots 1042 and short slots 1044) extend through the apertures of blade 1028 when assembled. This further contains and aligns blade 1028 but allows the blade to “float” relative to blade holder 1020. What is meant by “float” is that blade 1028 is loosely attached rather than rigidly attached to blade holder 1020 and a small amount of movement can occur between the two. The distal ends of short slots 1044 may be provided with rounded openings (as best seen in FIG. 76) for receiving the ends of pull pin 1030 (as best seen in FIG. 75.) This arrangement allows blade holder 1020 to indirectly drive blade 1028 in the proximal direction through pull pin 1030, which can be welded to blade holder 1020. As such, essentially all of blade 1028 is put into compression when in use. This allows blade 1028 to be actuated without being welded (or attached) directly to blade holder 1020 and distributes the load to prevent stress concentrations in blade 1028. In some embodiments, blade 1028 comprises tungsten carbide. Not welding (or attaching) directly to blade 1028 and distributing the pull force across blade 1028 prevents the tungsten carbide blade from shattering.

When blade 1028 moves proximally with respect to filler plate 1024 the tether is squeezed between blade 1028 and a backstop 1045 on the proximal portion 1046 of filler plate 1024. Backstop 1045 provides a hard surface for blade 1028 to engage with to ensure all filaments of the tether are cut.

When pull wire 1034 is tensioned, posts 1041 of blade holder 1020 (located between long slots 1040 and medium slots 1042) make contact with cutouts 1047 on proximal portion 1046 of filler plate 1024 to limit the motion of blade holder 1020 and blade 1028. This prevents additional force from being applied to blade 1028 after it has made contact with backstop 1045. In some embodiments, the interference between blade 1028 and backstop 1045 is 0.0015 inches. Blade 1028 will make contact with backstop 1045 but the minimal interference will allow the blade to “dig into” the back stop slightly, so as to fully cut the tether. The limited interference prevents additional buildup of force on blade 1028. As previously indicated, without this limited travel of blade holder 1020, blade 1028 would often shatter.

After filler plate 1024, blade 1028 and pull pin 1030 are assembled with blade holder 1020, upper stationary plate 1022 and lower stationary plate 1026 may be assembled with filler plate 1024 and the entire assembly inserted into the distal end of catheter 1012. As best seen in FIG. 76, the distal end of catheter 1012 may be provided with a pair of opposing slots 1048 for receiving the proximal portion 1046 of filler plate 1024. The proximal ends of stationary plates 1022 and 1026 (which in this exemplary embodiment are identical) may be providing with portions that extend inside the distal end of catheter 1012. Stationary plates 1022 and 1026 may include longitudinal slots 1049 (best seen in FIG. 77) configured to receive the longitudinal arms of blade holder 1020 and may serve to laterally guide blade holder. Laser or ultrasonic welding, adhesives, and or other fastening means may be used to fasten stationary plates 1022 and 1026 and or filler plate 1024 to one another and or to catheter 1012. Scalloped-shaped cutouts may be provided in the distal end of catheter 1012 as shown to provide room for a tether to pass through cutter assembly 1010.

A pair of vertically oriented slots 1050 may be provided in the proximal end of blade holder 1020 as shown for receiving pull wing 1032. Longitudinal slots 1052 may be provided through the top and bottom walls of catheter 1012 near its distal end for slidably receiving pull wing 1032. During assembly, pull wing 1032 may be slid through longitudinal slots 1052 of catheter 1012 and coupled to slots 1050 in blade holder 1020, such as by laser welding. Pull wing 1032 may also be attached to the distal end of pull wire 1034. This arrangement allows pull wire 1034 to pull the blade holder 1020 in the proximal direction as pull wing 1032 travels in slots 1052.

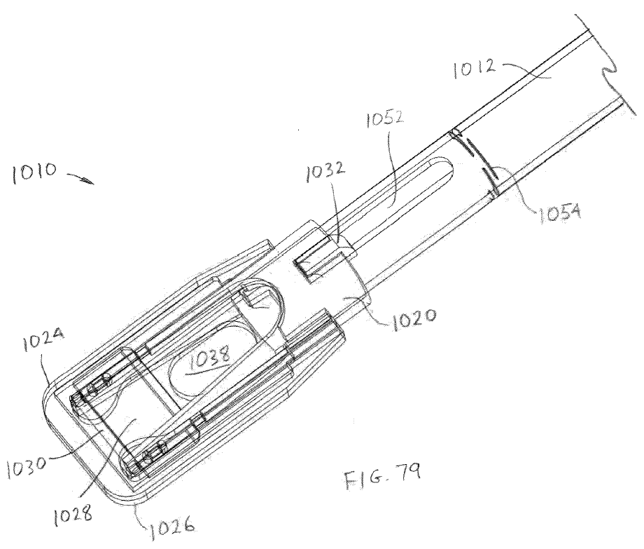

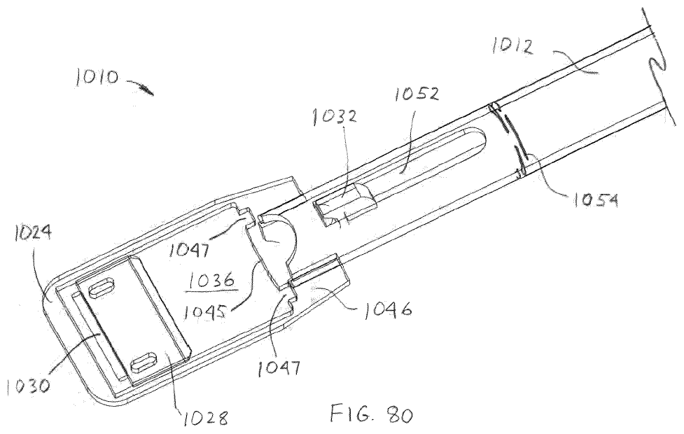

Referring to FIGS. 78-80, additional views of cutter assembly 1010 are provided. FIG. 78 is an enlarged transparent view showing the distal end of instrument 1000, FIG. 79 is a similar view with the upper stationary plate removed for ease of understanding, and FIG. 80 is another similar view with the upper stationary plate, the lower stationary plate and the blade holder removed for ease of understanding. Also shown is a laser cut section 1054 that increases the flexibility of that portion of catheter 1012, which has been found advantageous during use of this exemplary embodiment. As shown in FIG. 74, there are a total of three laser cut sections in catheter 1012, all relatively close to its distal end.

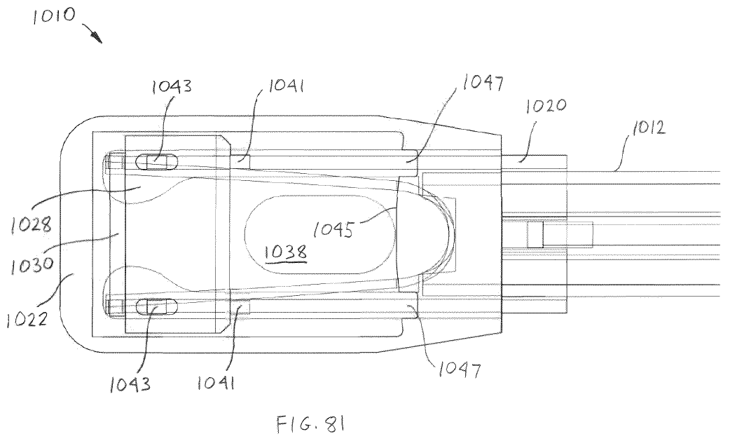

Referring to FIGS. 81-83, additional views of cutter assembly 1010 are provided. FIG. 81 is an enlarged transparent top plan view showing the distal end of instrument 1000 with cutting blade 1028 and blade holder 1020 in a distal position. FIG. 82 is similar view with cutting blade 1028 and blade holder 1020 in a distal position and upper stationary plate and lower stationary plate removed for ease of understanding.

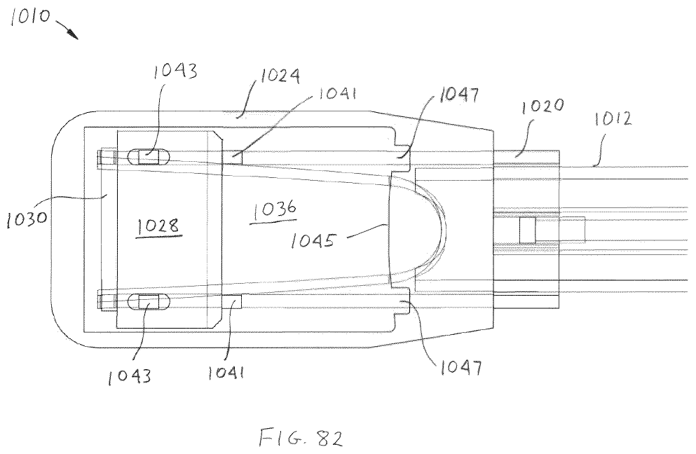

FIG. 83 is another similar view showing cutting blade 1028 and blade holder 1020 in a proximal position and upper stationary plate, lower stationary plate and pull pin removed for ease of understanding. As shown and previously indicated, blade 1028 is captivated by blade holder 1020 when posts 1043 pass through the elongated apertures in blade 1028. In this exemplary embodiment, the apertures in blade 1028 are larger in width and depth than posts 1043, thereby allowing blade 1028 to move in at least two directions (i.e., in the plane of blade 1028) relative to blade holder 1020. This “floating” arrangement ensures that as pull pin 1030 moves proximally against the distal edge of blade 1028 it contacts the blade evenly and distributes the pulling force evenly along blade 1028 to inhibit shattering the blade.

As previously mentioned, blade holder 1020 may include a stop configured to prevent the blade holder from moving further in a proximal direction once it has reached its desired proximal position and finished cutting the tether. In this exemplary embodiment, blade holder posts 1041 serve as stops when they contact the proximal surfaces of cutouts 1047 in filler plate 1024. Blade 1028 may be configured to contact backstop 1045 before stops 1041 prevent blade holder 1020 from moving further in the proximal direction. In this exemplary embodiment, stops 1041 are configured to allow blade 1028 to move further in the proximal direction at least an additional 0.0015 inches after the blade has contacted the backstop. In other embodiments, there may be more than 0.0015 inches of interference, less than 0.0015 inches of interference, no interference, or a small gap that exists between blade 1028 and backstop 1045 when stops 1041 hit cutouts 1047 and prevent further proximal movement, as shown in FIG. 83. In some embodiments, the distal surfaces of stops/posts 1041 may further serve to limit the floating movement of blade 1028 relative to blade holder 1020 in order to keep blade 1028 better aligned with pull pin 1030.

Backstop 1045 may be provided with a convex shape to aid in reliable tether cutting, as shown in FIGS. 81-83. In some embodiments, the convex shape comprises one or more radiuses of at least 0.070 inches. In other embodiments, the convex shape is formed by a flat center portion taking up no more than half of the width of backstop 1045, with the remaining side portions tapering and/or curving away from the flat center portion. In some embodiments, the side portions include flat portions that taper away from the center portion by at least 5 degrees.

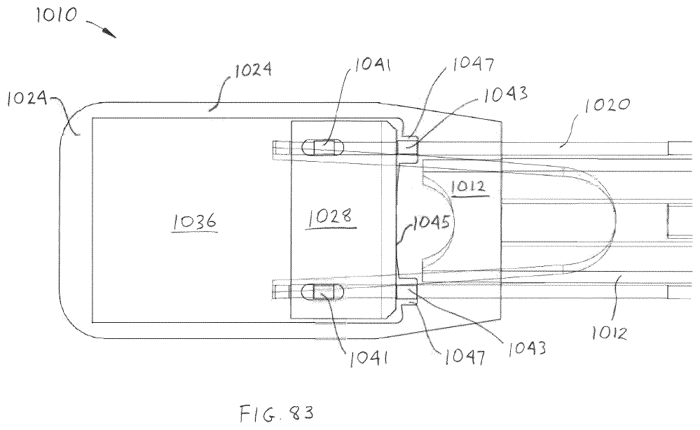

Referring to FIG. 84, an enlarged transparent side view is provided showing an exemplary cutting blade 1028 and pull pin 1030. In this exemplary embodiment, cutting blade 1028 includes a cutting edge 1056 that comprises a flat center portion surrounded by tapered surfaces that each comprise an angle A, as shown, relative to the top and bottom surfaces of cutting blade 1028. In some embodiments, angle A is at least 15 degrees and or is less than 20 degrees. In other embodiments (not shown), cutting edge 1056 does not include a flat center portion, but includes tapered surfaces that meet at a sharp apex. In some of these embodiments, the sharp apex is eased with a radius of about 0.0005 inches. In some embodiments, cutting blade 1028 has a length of no more than about 0.150 inches, a width of no more than about 0.080 inches, and a thickness of no more than about 0.012 inches.

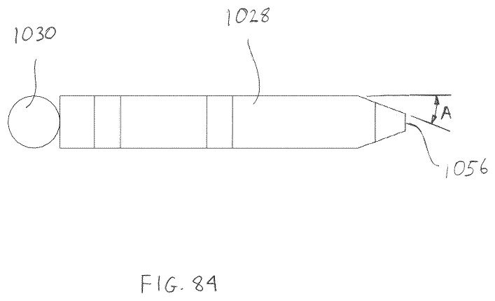

Referring now to FIGS. 85 and 86, details of the proximal handle section 1014 of tether cutting instrument 1000 are shown. FIG. 85 is a rear side view and FIG. 86 is an exploded perspective view of handle section 1014. As best seen in FIG. 85, handle section 1014 may include a forked lower section 1060 provided with a pair of inwardly facing press-fit stainless-steel ball-nose spring plungers 1062. This arrangement allows handle section 1014 to be temporarily attached to a rail carriage 770 (shown in FIG. 48F) during use.

As best seen in FIG. 86, in this exemplary embodiment handle section 1014 includes a left handle housing 1064, a right handle housing 1066, an actuator 1016, a spring 1068, a cable lock 1070, a set screw 1072 and a flush port 1018. Also shown in FIG. 86 are the proximal ends of catheter 1012 and pull wire 1034. A pair of outwardly facing circular bosses 1074 may be provided at the top of actuator lever 1016, configured to be received in mating recesses 1076 such that lever 1016 is sandwiched between handle housings 1064 and 1066 and pivots with respect to recesses 1076. Cable lock 1070 may be inserted into bore 1078 through lever 1016 to receive the proximal end of pull wire 1034. Set screw 1072 may then be threaded into cable lock 1070 to lock pull wire 1034 relative to lever 1016 such that when the lever is squeezed, pull wire 1034 is pulled proximally to actuate the cutter. Spring 1068 may be provided in handle housing slot 1080 and a slot in the back of lever 1016 (not shown) such that lever 1016 and cutting blade 1028 (shown in FIG. 78) are returned to their distal starting positions when lever 1016 is released. Flush port assembly 1018 serves to seal the proximal end of catheter 1012 where pull wire 1034 exits, and is configured to permit periodic flushing of instrument 1000.

The above-described tether cutting instrument is suitable for cutting multiple tethers of the type also described above. In other words, while the instrument may be constructed to be disposable after use in a single surgical procedure, only one instrument may be needed during the surgical procedure to cut two or more tethers in sequence.

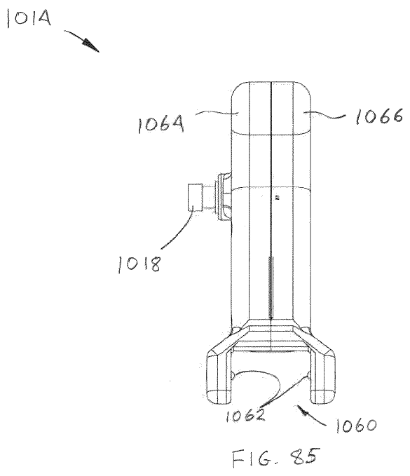

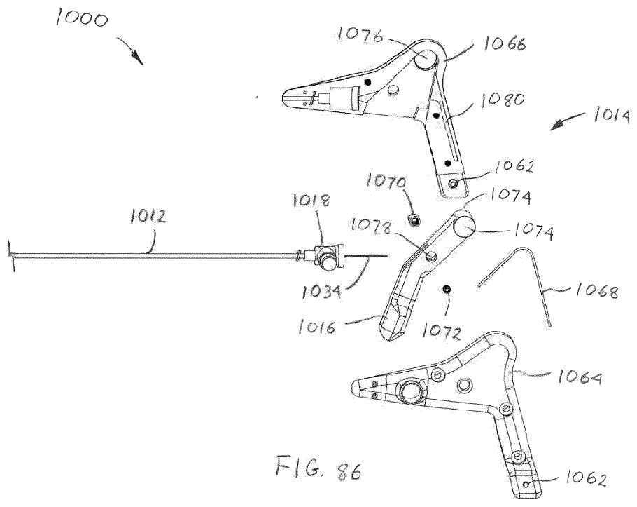

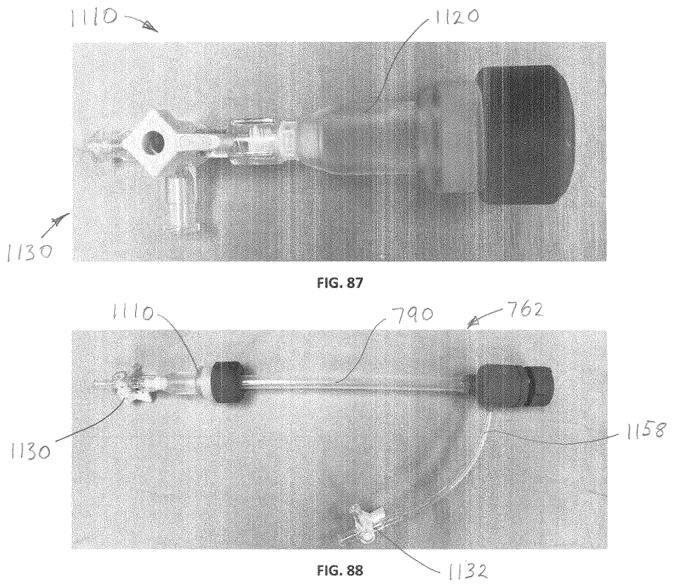

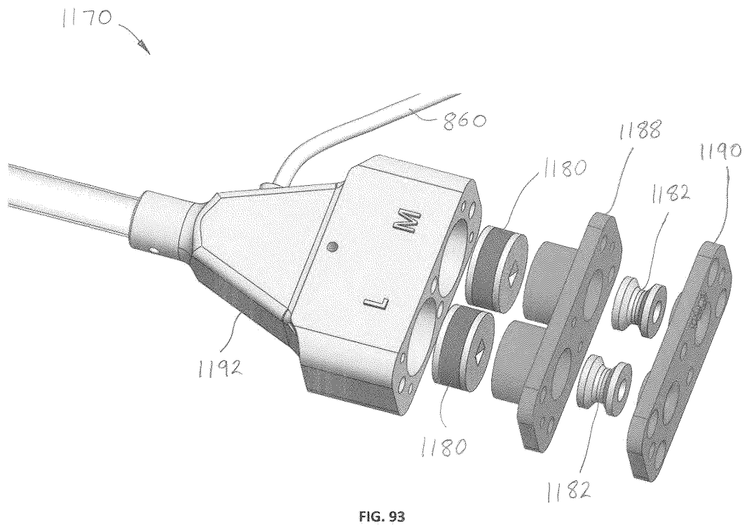

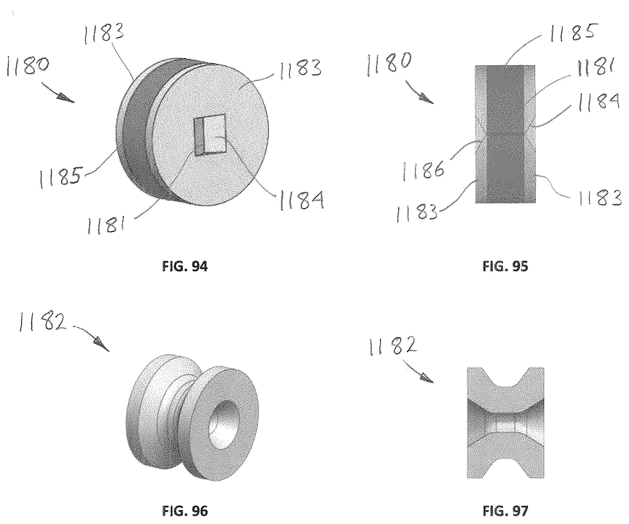

Referring to FIGS. 87-105, exemplary systems, devices and methods for flushing surgical instruments and providing for hemostasis are provided. These address a number of challenges that may arise in the use of prior art surgical devices and or some of the instruments previously described herein. For example, when using such devices in a surgical procedure, it can be challenging to remove all of the air from the instruments, to achieve hemostasis so that a significant amount of blood from a patient being operated upon does not leak out from the devices, to prevent additional air from entering the devices during a procedure and passing into the patient, and to not require operating room personnel to spend an inordinate amount of time in preparing the surgical instruments to achieve these objectives. These challenges can become particularly acute when flushing large, multi-lumen systems or systems having a complex implant inside of a loading tool, when needing to introduce a large, open-ended system into an outer guide without trapping air inside, when sealing over a small diameter tether in a large diameter valve in the outer guide, when converting a single hemostasis valve to a double (or more) hemostasis valve during procedure, or when sealing a variety of sizes, shapes, rough, and/or side-by-side lumens.

Referring first to FIG. 87, an exemplary flush tool 1110 constructed and used according to aspects of the disclosure is shown. To aid in flushing the instrumentation systems, removing bubbles, and eliminating bubbles from returning, flush tool 1110 was developed by the applicants to control fluid, particularly at the distal end of implant loading tool 762. Flush tool 1110 is configured to slide over the distal end of the loading tool tube 790, as shown in FIG. 88. In this exemplary embodiment, a silicone sleeve provides a fluid tight seal over the loading tool tube 790. Flush tool 1110 has an elongated shape to allow tether pullers 694 to extend out of the distal end of loading tool 762 and into a clear cavity 1120 of flush tool 1110. There is a fluid port 1130 on the distal end of flush tool 1110. Typically, systems are flushed by introducing fluid from the proximal end and elevating the distal end to fill the system with fluid and push air out of the distal end. But with the complex instrumentation designs described above, there are areas where air bubbles can remain in the system. (The surface tension of the aqueous solutions, usually saline, can produce air bubbles that are difficult to remove). Flush tool 1110 provides the ability to reverse the flow of fluid from the distal to proximal direction. It also provides the ability to pull a vacuum from the distal end (and in some embodiments from the proximal end), and it maintains fluid in the system when left horizontally. Reversal of flow during the flushing process can cause bubbles to be released and flushed from the system. Pulling a vacuum at the distal end can also aid in releasing bubbles trapped in the area of the loaded implant and exposed drive components. After the system is flushed it may be connected to an intravenous-type drip and laid in a horizontal orientation until the physician is ready to use it. Flush tool 1110 maintains fluid in loading tool 762 that would otherwise drain when the loading tool is in the horizontal direction. Flush tool 1110 also allows for transfer of the system to the operating table without loss of fluid.

In some embodiments, the loading tool 762 also has a flush valve 1132 coupled to its proximal end that can provide additional flow patterns for releasing trapped air bubbles. The inner surface of loading tool 762 can be made from or coated with a low surface energy material, or may be treated to render it hydrophilic, so as to inhibit bubbles from being trapped along the inside diameter of the loading tool. In addition, one or more flush ports may be added to other locations within the instrumentation system to provide improved access for flushing. After flushing the instrumentation, stands (not shown) may be used to elevate the distal end of the system. This arrangement can assist in maintaining a fluid column in the system, and can help manage the devices on the prep table before they are used in a surgical procedure.

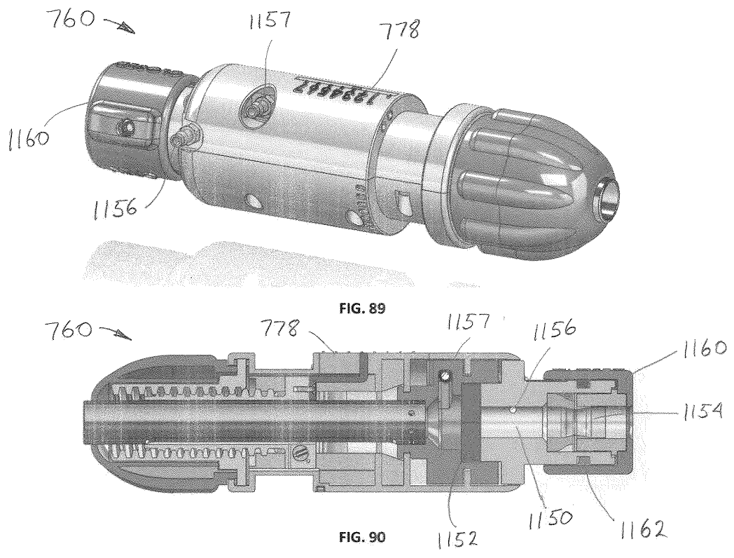

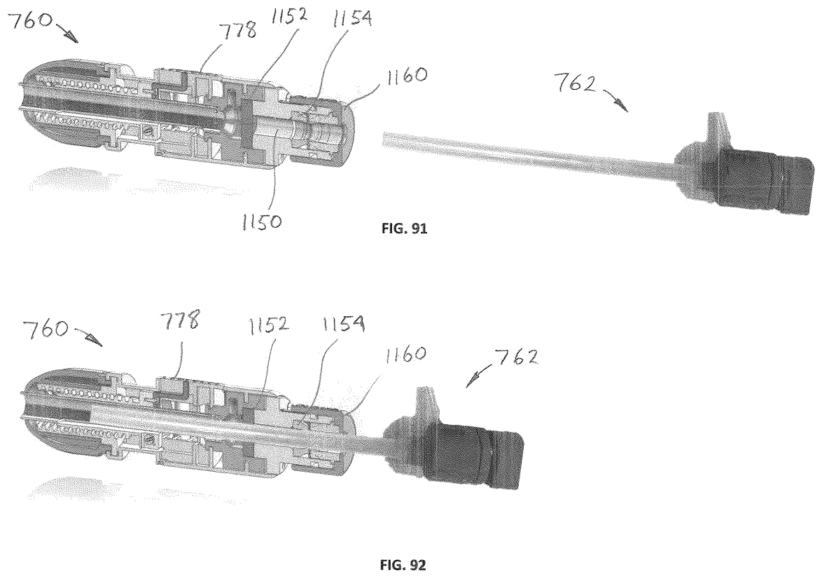

Referring to FIGS. 89-92, flushing/hemostasis features that may be provided on the proximal handle assembly 778 of an outer steerable catheter assembly/outer guide 760 will be described. As previously described, in some embodiments the annuloplasty system has a large loading tool 762 over the implant. To allow the implant to be pushed through the loading tool, the tool is open on its distal end. With the loading tool 762 being an open system, fluid can drain from it (and therefore air can be introduced into the system). Typically, physicians will drip fluid over the back of an outer guide to create a “wet” connection when inserting an inner guide. With larger systems, such as those described herein, this process is not well controlled, and it can be easy to trap air in the system during this insertion process. According to aspects of the present disclosure, this issue may be addressed by incorporating a flushing chamber 1150 between a closed distal valve 1152 (such as a single slit) and open hourglass valve 1154 within outer guide handle 778 (as best seen in FIG. 90). In the exemplary embodiment shown, the hourglass valve 1154 is open when empty (FIG. 91) but is sized to passively seal over the loading tool 762 when the tool is introduced into outer guide handle 778 (FIG. 92). In this embodiment, when loading tool 762 is being introduced, fluid is constantly flushed into chamber 1150 through port 1156 and flows out of hourglass valve 1154. With enough flow, a bolus of fluid is created beyond the proximal end of the outer guide handle 778 to allow the distal tip of loading tool 762 to make contact with this bolus of fluid before entering hourglass valve 1154 and creating a fluid tight seal, preventing trapped air in the system. An additional port 1157 (best seen in FIG. 90) may be provided in handle assembly 778 distal to single slit valve 1152 to aid in flushing outer guide 760 between its distal end (not shown) and valve 1152.

In addition, a port may be added to loading tool 762, such as port 1158 shown in FIG. 88, to allow aspiration at a more beneficial location than aspiration port 1156 on outer guide 760 (FIG. 90). Typically, aspiration is from an outer guide port, but in this exemplary embodiment loading tool 762 extends beyond it (as shown in FIG. 92), thereby reducing fluid flow through the outer guide port. Port 1158 on loading tool 762 can directly aspirate the area around the implant as well as inside the outer guide 760 distal to loading tool 762. Also, with the angle of the stabilization system in some embodiments (as shown in FIGS. 48A, 48C-F and 73), port 1158 on loading tool 762 is at higher elevation to better capture and expel air from the system.

It is desirable to have outer guide 760 hemostasis valves that seal in all situations, such as when outer guide handle assembly 778 is empty (as depicted in FIG. 91), when loading tool 762 is received within handle assembly 778 (as depicted in FIG. 92), and when one or more tethers 614 (as depicted in FIG. 57) extend through handle assembly 778. Typically, large hemostasis valves have challenges in sealing over a small diameter member such as a guide wire, or in this case a tether. This is especially true when a side load is applied to a wire or tether (perpendicular to the hemostasis valve), causing it to open and leak. Multiple small diameter members make sealing even more challenging. This issue is further exasperated because, in most implementations, the devices disclosed herein operate on the venous side of the circulatory system which can have a low blood pressure and, in some cases, create a negative pressure at the height of outer guide handle assembly 778 where the hemostasis valves are located. Sealing effectively with a negative pressure is more challenging than with positive pressure. With positive pressure, the viscosity of the blood reduces flow through potential leak paths and small leaks or drips of blood are not clinically relevant. But with negative pressure, air could be pulled into the system. What might be a small leak for a liquid could allow much more volume of air to pass through given the low viscosity of air. The introduction of air into the system can be clinically significant due to the potential for air embolism, which can be a serious and potentially fatal condition.

As previously described herein, after the first implant(s) are placed during a surgical procedure and the inner guide(s) are retracted, tether(s) remain in place through the outer guide hemostasis valve. The inventive arrangement shown in FIGS. 89-92 addresses the above challenges by incorporating a robust single slit distal valve 1152 and an active compressible hourglass valve 1154. The robust single slit valve 1152 provides hemostasis when the outer guide is empty, and the hourglass valve 1154 is not activated. In this exemplary embodiment, single slit valve 1152 is made of relatively thick and low durometer silicone. For example, the thickness may be 6 mm and the valve may have a durometer of 20A. In some embodiments, the thickness is between 4 and 12 mm inclusive, and the valve material may have a durometer in the range of 10-30 A. In the exemplary embodiment disclosed, these characteristics allow it to seal over the tethers. The thickness and low durometer of valve 1152 could present challenges for a full inner guide system passing directly through it and needing to be manipulated in the heart. However, when used with the previously described instrumentation, only loading tool 762 is passed directly through valve 1152 and it is only advanced a short distance through the single slit, thereby allowing much high forces to be applied compared to advancing a full system directly through valve 1152. This is because the inner guide systems that are advanced all the way into the heart and need to be manipulated there extend through loading tool 762 during operation. The seal on loading tool 762 then provides a sliding hemostasis seal for the inner guide systems while they are being manipulated in the heart but does not interfere with the advancement or manipulation of these inner guide systems.

In this exemplary embodiment, the proximally located hourglass valve 1154 may be compressed by rotating housing 1160 that rides in a helical channel 1162. When compressed, valve 1154 closes tightly, including over any tethers passing through it. While the single slit valve 1152 provides an adequate seal over tether(s) when actively removing inner devices, hourglass valve 1154 provides a more secure seal over an extended period of time, such as while the next inner device is being transferred to the instrument operating table. Hourglass valve 1154 in its open position also provides a redundant seal over loading tool 762. In some embodiments, compression applied perpendicular to single slit valve 1152 (i.e., perpendicular to its central axis) can improve its performance by keeping the slit closed.

For more complex surgical procedures, especially structural heart procedures, it can be desirable at times during the procedure to manipulate two or more devices independently through an outer guide. This provides a challenge for maintaining hemostasis on both single large bore devices and smaller multiple systems passing through the same valve. With the exemplary instrumentation systems discussed above, the implant catheters may be placed with systems that use the full diameter of outer guide 760, but the lock and cutter systems are advanced concurrently and manipulated separately. With prior art valve arrangements, it is not possible to achieve proper hemostasis while passing the various instruments above through the outer guide. To address these challenges, the applicant of this patent application has developed a bifurcated hemostasis tool, which will now be described.

Referring to FIGS. 93-98, an exemplary bifurcated hemostasis tool 1170 is provided. Tool 1170 allows the hemostasis of the entire instrument system to be converted to two or more discreate hemostasis valves or paths during the surgical procedure. To work with tethers 614 (depicted in FIG. 57), a preloaded two lumen tool (such as tool 862 shown in FIGS. 62D-62F) may be preinserted in bifurcated hemostasis tool 1170 to assist with threading the tethers through tool 1170. In this exemplary embodiment, loading tool 862 is removed from hemostasis tool 1170 before tool 1170 is inserted into an outer guide.

The multiple hemostasis valves of exemplary bifurcated hemostasis tool 1170 meet the following requirements:
1. Seal when empty;
2. Seal over tether(s);
3. Allow passage, in both directions, of an irregularly shaped lock and cutter with reasonable force and without damage to the valve; and
4. Seal over a tether cutter instrument shaft and tether when they are side-by-side.

Similar to the challenges and solutions of the outer guide hemostasis system described above with reference to FIGS. 89-92, a single slit valve 1180 in combination with an hourglass seal 1182 is used in each of the two paths. In this exemplary embodiment, hourglass seal 1182 is not an “active” hourglass valve (unlike active hourglass valve 1154 described above). Slit valves 1180 provide a hemostasis seal over tethers, and hourglass valves 1182 seal over the shaft of a lock installing and tensioning instrument and over the shaft of a tether cutting instrument.

A challenge encountered when designing slit valve 1180 was providing the ability to pass irregularly shaped locks and cutters through the valve with reasonable force and without damage to the valve. To overcome this challenge in the exemplary embodiment shown in FIGS. 93-98, slit seal 1180 was formed by thin outer layers 1183 of higher durometer silicone on the exterior surfaces (as best seen in FIG. 95) to prevent tearing when introducing locks and cutters through slit 1181, and a lower durometer material 1185 on the interior of valve 1180 to provide proper sealing. In some embodiments, the interior of valve 1180 is made of silicone having a durometer of 10A, and the thin outer layers 1183 have a durometer of 45A. Beveled entrances 1184 and exits 1186, aligned with rectangular profiles of the locks and cutters, facilitate their passage through valves 1180. In some embodiments, valves 1180 and 1182 are coated with 100% Parylene to increase their hydrophilic and/or antimicrobial properties.

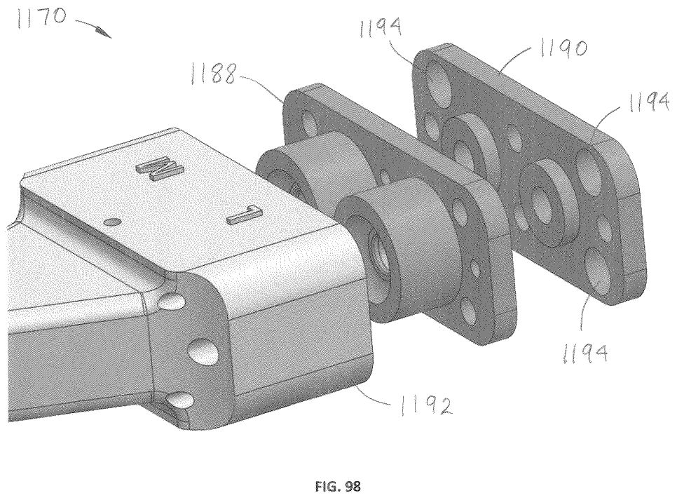

Independent compression plates 1188 and 1190 may be provided as shown in FIGS. 93 and 98 to adjust the sealing characteristics of valves 1180 and 1182, respectively. In this exemplary embodiment, four attachment and adjustment screws (not shown) may be used to compress valves 1180 between plate 1188 and housing 1192 to make these valves stiffer. Similarly, four other attachment and adjustment screws (not shown) may be used to compress valves 1182 between plate 1190 and plate 1188 to make these valves stiffer. Inner compression plate 1188 may be adjusted after assembly of tool 1170 through clearance holes 1194 located in the corners of plate 1190 (as shown in FIG. 98). Flush port 860 may be provided in housing 1192 as shown in FIG. 93 to remove air and prefill bifurcated hemostasis tool 1170 with fluid before introducing it into an outer guide, in a manner similar to that previously described relative to introducing implant loading tools into an outer guide.

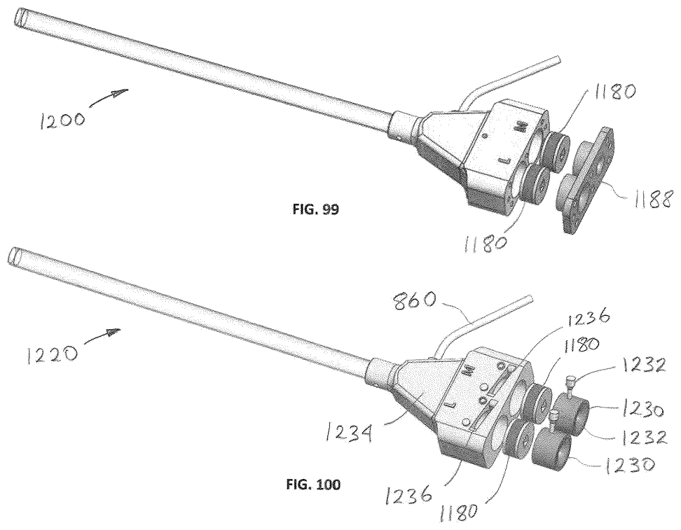

Referring to FIG. 99, another exemplary bifurcated hemostasis tool 1200 is provided. Tool 1200 is similar to tool 1170 described above, but each path through tool 1200 has a single seal comprising a single slit valve 1180, and utilizes a single compression plate 1188.

Referring to FIGS. 100-104, another exemplary bifurcated hemostasis tool 1220 is provided. Tool 1220 is similar to tool 1200 described above, but a pair of rotating compression cylinders 1230 are used instead of a compression plate to adjust the characteristics of single slit valves 1180. With this arrangement, an operator can actively adjust the compression on seals 1180 during an operation such that the force required to introduce a lock or cutter instrument is reduced, yet a tight seal can be maintained on the instrument after it has been introduced.

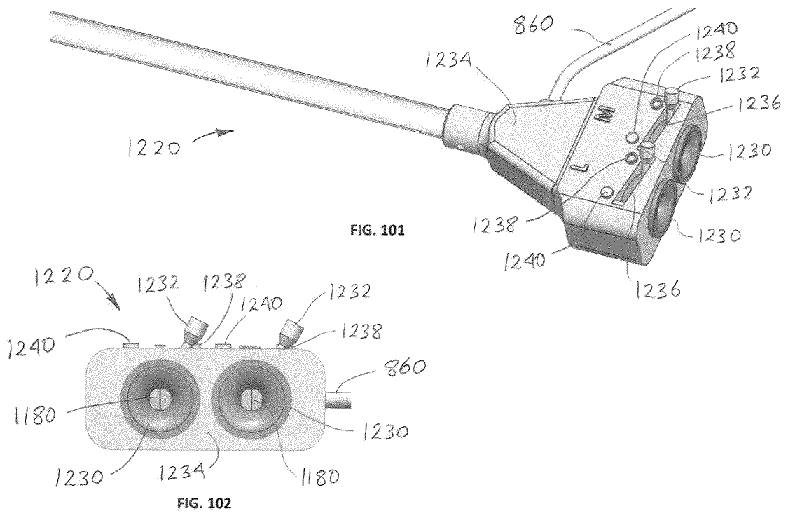

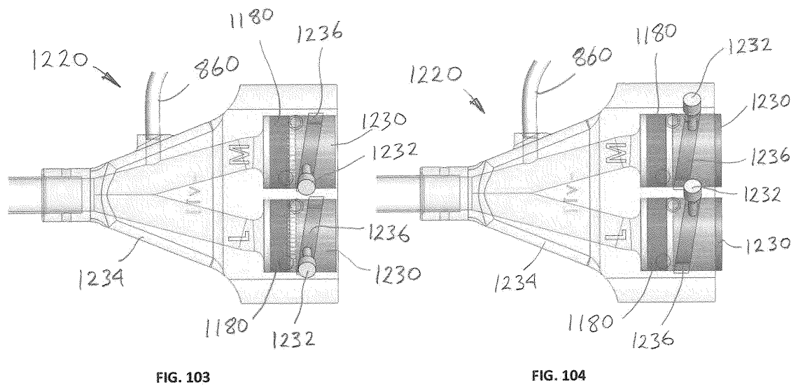

In this exemplary embodiment, radially extending knobs 1232 are provided on compression cylinders 1230 for actuating the cylinders. Upon assembly with housing 1234, knobs 1232 extend upwardly from cylinders 1230 through slots 1236 in housing 1234. Slots 1236 are angled such that when knobs 1232 are rotated, they drive cylinders 1230 in a distal direction, thereby compressing seals 1180. As best seen in FIG. 102, knobs 1232 can be rotated in a counterclockwise direction from a valve “open” position to a valve “closed” position. In some embodiments, the slits of valves 1180 remain closed in either position, but the open/closed terminology may be used to help an operator visualize the state of the valves. “Open” and “Closed” indicators 1238 and 1240, respectively, may be provided on the top surface of housing 1234 next to slots 1236, as shown in FIGS. 100-102, to indicate the state of each valve 1180. The indicators may be symbols, text, colors, and/or other indicia capable of depicting the state of each valve 1180. In this exemplary embodiment, each compression cylinder 1230 may be operated individually and may be infinitely variable between the fully open position (depicted in FIGS. 101, 102 and 104) and the fully closed position (depicted in FIG. 103). In operation, knob(s) 1232 may temporarily be moved to the Open position to aid in the insertion of instrument(s) through the associated valve 1180, and then moved to the Closed position to better seal over a tether, lock instrument shaft, cutter instrument shaft or combinations thereof, such as a cutter shaft alongside of a tether. In some embodiments, a valve 1180 may remain in the Closed position when a tether cutter is being introduced through the valve. As with previously described embodiments, a flush port 860 may be provided in housing 1234.

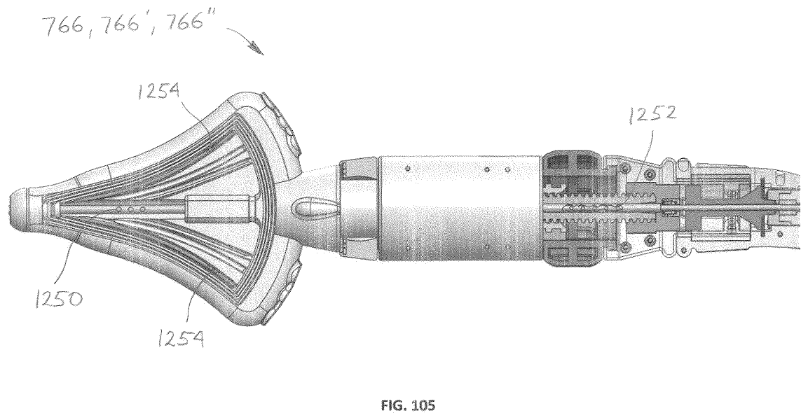

Referring to FIG. 105, additional features may be added to the handle of the inner steerable catheters 766, 766′ and 766″ to aid in flushing them and expelling air. Vent holes 1250 may be formed (such as by laser cutting) along a length of the central bore in the wing section of inner steerable catheter as shown. This arrangement of holes allows air and or a small amount of fluid to escape from the central bore to ensure air does not migrate distally toward the patient. This process is facilitated when a positive fluid pressure is applied to the system, such as with a syringe or a constant IV drip connected to the instrumentation. A series of similar vent holes 1252 may also be provided more proximally on the central bore, such as in the torque driver section as shown, and or a series of holes 1254 along the tether routing tubes as also shown. In some embodiments, a series of 3 or 4 holes in each location have been found to work well.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present disclosure.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about”, “approximately” or “generally” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the disclosure as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the disclosure as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A flushing and hemostasis apparatus for a trans-catheter surgical procedure, the apparatus comprising:
an elongated body having a proximal end, a distal end, and a mid-section located between the proximal end and the distal end;
a seal assembly located at the proximal end of the elongated body and configured to receive a distal end of an implant loading tool therethrough; and
a valve assembly located at the distal end of the elongated body and comprising a flush port, the valve assembly configured to move between an open position in which the flush port is in fluid communication with the mid-section of the elongated body and a closed position in which the distal end of the elongated body is sealed off from the mid-section,
wherein the mid-section is configured to receive an element protruding from the distal end of the implant loading tool.

2. The apparatus of claim 1, wherein at least a portion of the mid-section of the elongated body is transparent.

3. The apparatus of claim 1, wherein the mid-section is configured to receive a tether puller protruding from the distal end of the implant loading tool.

4. The apparatus of claim 1, wherein the seal assembly comprises a rotatable collar that is configured to alternately tighten and loosen a seal around the distal end of the implant loading tool.

5. The apparatus of claim 1, wherein the valve assembly is configured to allow air and fluid from the implant loading tool to flow out of the flush port.

6. The apparatus of claim 1, wherein the valve assembly is configured to be attached to a vacuum source to allow air and fluid to be drawn out of the implant loading tool.

7. The apparatus of claim 1, wherein the valve assembly is configured to be attached to a fluid source to allow fluid to be introduced into the implant loading tool.

8. An outer guide configured for a trans-catheter surgical procedure, the outer guide comprising:
a proximal handle having a proximal end, a distal end, and a mid-section located between the proximal end and the distal end, wherein the proximal end and mid-section are configured to receive a distal end of an implant loading tool therein;
a flushing chamber located within the proximal handle;
a closed distal valve located within the proximal handle distal to the flushing chamber, the distal valve configured to seal against the distal end of the implant loading tool when received therethrough and configured to remain closed when the implant loading tool is not received therethrough;
an open proximal valve located within the proximal handle proximal to the flushing chamber, the proximal valve configured to seal against the distal end of the implant loading tool when received therethrough and configured to remain open when the implant loading tool is not received therethrough; and
a flushing port located on the proximal handle and in fluid communication with the flushing chamber, thereby allowing fluid to be constantly flushed into the flushing chamber through the flushing port and flow out of the open proximal valve when the implant loading tool is being introduced into the proximal handle to create a bolus of fluid beyond the proximal end of the proximal handle to allow the distal tip of the implant loading tool to make contact with the bolus of fluid before entering the proximal open valve, inhibiting air from being trapped in the outer guide or the implant loading tool.

9. The outer guide of claim 8, wherein the closed distal valve is a single slit valve.

10. The outer guide of claim 8, wherein the open proximal valve is an hourglass-shaped valve.

11. The outer guide of claim 8, further comprising a second flushing port located on the proximal handle and in fluid communication with a central bore of the proximal handle at location that is distal to the closed distal valve.

12. The outer guide of claim 8, wherein the open proximal valve comprises a rotatable housing that permits the proximal valve to be tightly compressed, including over any tethers passing through it, thereby providing a redundant and more secure seal than the closed proximal valve.

13. A flushing and hemostasis system comprising:
the outer guide of claim 8; and
an implant loading tool configured to receive a surgical implant in a central bore of the implant loading tool and to allow the implant to be pushed out of a distal end of the loading tool and into the outer guide by a steerable inner catheter, thereby allowing the steerable inner catheter to push the implant through the entire length of the outer guide and manipulate the implant inside a patient without the implant or the steerable inner catheter coming into contact with any hemostasis valves in the outer guide.

14. A bifurcated hemostasis tool comprising:
a housing having a proximal end and a distal end;
a single tube extending from the distal end of the housing;
at least two bores located through the proximal end of the housing, each of the bores converging together and being in fluid communication with the single tube;
a single hemostasis valve located in each of the bores.

15. The tool of claim 14, wherein each of the valves is a single slit valve.

16. The tool of claim 15, wherein each of the valves comprises an interior portion, a proximal exterior layer and a distal exterior layer, wherein the proximal exterior layer and the distal exterior layer are each thinner than the interior portion and each comprise a material having a higher durometer than that of a material of the interior portion.

17. The tool of claim 16, wherein the proximal exterior layer and the distal exterior layer of each of the valves comprises a pair of beveled surfaces aligned with a slit of the interior portion.

18. The tool of claim 17, wherein the interior portion, the proximal exterior layer and the distal exterior layer of each of the valves is formed of silicone.

19. The tool of claim 18, wherein each of the valves has a coating that comprises Parylene.

20. The tool of claim 15, wherein each of the valves is provided with a rotating compression cylinder, wherein each compression cylinder is movable between a first position in which one of the valves is compressed so that it is stiffer and a second position in which the valve is uncompressed or less compressed and is less stiff.

21. The tool of claim 20, wherein each of the compression cylinders comprises a radially extending knob configured to rotate an associated compression cylinder.

22. The tool of claim 20, wherein each of the compression cylinders may be operated individually.

23. The tool of claim 20, wherein each of the compression cylinders may be infinitely variable between the first position and the second position.

24. The tool of claim 14 further comprising a flush port located on the housing.

25. A method of flushing a surgical instrument system and achieving hemostasis, the method comprising;
providing an outer guide, an inner steerable catheter, an implant loading tool slidably coupled to the inner steerable catheter, and a flushing tool, the outer guide comprising a closed distal valve, an open proximal valve and flushing chamber located therebetween;
placing the flushing tool over a distal end of the implant loading tool;
filling the inner steerable catheter, the implant loading tool and the flushing tool with a sterile fluid;
closing a valve located at a distal end of the flushing tool;
introducing a sterile fluid into the flushing chamber so that it flows out of the open proximal valve to create a bolus of fluid beyond a proximal end of the outer guide;
removing the flushing tool from the distal end of implant loading tool; and
introducing the distal tip of the implant loading tool to the bolus of fluid before introducing it through the proximal open valve, thereby inhibiting air from being trapped in the outer guide or the implant loading tool during insertion of the implant loading tool into the outer guide.

26. The method of claim 25, further comprising introducing the distal tip of the implant loading tool through the closed distal valve.

27. The method of claim 26, further comprising using the inner steerable catheter to push a surgical implant out of the implant loading tool and through the outer guide until the implant emerges from a distal end of the outer guide and into a patient.

28. The method of claim 27, further comprising manipulating the implant within the patient with the inner steerable catheter without the inner steerable catheter contacting the closed distal valve or the open proximal valve.

29. The method of claim 25, further comprising providing a series of vent holes along at least one bore of a proximal handle of the inner steerable catheter.

30. The method of claim 25, further comprising:
removing the inner steerable catheter and the implant loader from the outer guide; and
introducing a distal end of a bifurcated hemostasis tool through the open proximal valve and the closed distal valve.

31. The method of claim 30, further comprising, between the steps of removing the inner steerable catheter and introducing the distal end of the bifurcated hemostasis tool:
introducing a sterile fluid into the flushing chamber of the outer guide so that it flows out of the open proximal valve to create a bolus of fluid beyond a proximal end of the outer guide; and
introducing the distal end of the bifurcated hemostasis tool to the bolus of fluid before introducing it through the proximal open valve, thereby inhibiting air from being trapped in the outer guide or the bifurcated hemostasis tool during insertion of the bifurcated hemostasis tool into the outer guide.

32. The method of claim 31, further comprising, before introducing the distal end of the bifurcated hemostasis tool to the bolus of fluid:
flushing air out of the bifurcated hemostasis tool and filling it with a sterile fluid.
